(19) United States
(12) Patent Application Publication
(10) Pub. No.: US 2004/0131658 A1
(43) Pub. Date: Jul. 8, 2004

(54) Identification of diet-regulated disease-associated genes

(76) Inventor: James Kaput, Justice, IL (US)

Correspondence Address: Adam Warwick Bell, D. Phil., Suite 100, 416 Funston, San Francisco, CA, 94118, US

(21) Appl. No.: 10/700,305

(22) Filed: Oct. 31, 2003

Related U.S. Application Data
(60) Provisional application No. 60/423,104, filed on Nov. 1, 2002.

(51) Int. Cl. C12Q 1/68; A61K 47/00

(52) U.S. Cl. 424/439; 435/6

(57) ABSTRACT

The invention encompasses identification of diet-regulated disease-associated genes and EST's and methods of use in disease screening, monitoring and treatment. It also encompasses methods for formulating medical foods used to treat and prevent disease and slow disease progression. A new method of identifying diet-regulated disease-associated genes and ESTs is disclosed that employs gene expression analysis in inbred strains of mice and matching the results with publicly available QTL (quantitative trait locus) information.

The present application claims the benefit of U.S. provisional application Serial No. 60/423,104 filed Nov. 1^{st} 2002, which application is incorporated by reference herein.

The work presented herein has been partially funded by Illinois-Missouri Biotechnology Alliance (a USDA program, 94-34346-1795) and a American Institute for Cancer Research (NC93B63) funded CRADA with the National Center for Toxicological Research (Protocol E0260301)

### BACKGROUND

Diet plays a major role in disease causation and progression. At its most extreme level, starvation and nutritional deficiency disrupts metabolism so as to cause illness and death. At a more subtle level, many disease-related genes are regulated at some level by nutritional factors. The chemicals present in food (nutrients) may regulate gene expression at the transcription or translational level, at the level of post-translational modification, degradation or enzymatic activity. Glucose regulation of insulin and glucagon is a well-known example of diet-regulated enzyme regulation. The amount of a nutrient present in the body influences the equilibrium of metabolic pathways. In one very simple form, this can be thought of as a simple mass-action effect where the concentration of a metabolite (reactant) on the left hand side of an equation will drive the equilibrium to the right hand side, e.g., A+B [symbol] C+D. If the concentration of reactant A increases, all other factors remaining constant, the equilibrium will shift to the right. But unlike this simple example, metabolic pathways are often very complex, replete with redundancy, and are interrelated with many other metabolic pathways. Therefore, altering the concentration of a single metabolite often has pleioropic effects on highly disparate areas of physiology, including disease-related physiology. Hormones and other types of cell-signaling molecules, for example, are well-understood to be regulated by diet, and are equally well-known to be related to disease. There is also considerable evidence that total calorie intake may influence diseases including breast cancers, such as breast cancer.

Diabetes is a well-known example of a nutrient-regulated disease in which nutrient-regulated messengers (insulin and glucagon) play a key role. Obesity is another well-known diet related disease in which nutrient-regulated messengers such as leptins, adiponectin, and resistin are important control factors. The Center for Disease Control has declared obesity and Type 2 diabetes epidemic in the U.S and in the western world. These conditions, and many other chronic diseases, are caused by multiple genes influenced by many environmental factors. The complexity of these diseases makes them difficult to diagnose and treat. Specifically, three key factors may affect the diagnosis and treatment of chronic diseases:

a) The same disease phenotype may result from disturbance in different metabolic pathways

b) The genetic makeup of each human differs, causing variation in response to the same factors

c) Environmental factors, such as diet, influence health and disease development

Chronic diseases, including obesity, Alzheimer's, diabetes, cardiovascular diseases, and certain cancers (among others), are generally produced by the interplay of environmental factors and genetic mechanisms. In addition, different members of the population showing clinical symptoms of any given disease can be grouped with each group having some unique genes or ESTs that contribute to disease formation. Furthermore, subsets of the unique and commonly-distributed genes are regulated directly or indirectly by foods chemicals. Genes that are diet-regulated and involved in disease processes can be identified and grouped to provide diagnostic markers and targets for drugs.

The Problems at Hand

a) The same disease may result by disturbance of different pathways in the body. A large body of research has shown that a chronic disease may be caused by different pathways [1]. For example, tumors from different individuals may appear similar, but may be produced by mutations in different genes. Similarly, the clinical term “diabetes” encompasses a variety of different symptoms and causes all having to do with imbalances in insulin levels and sugar metabolism. Since several biological processes (or pathways) affect insulin production or serum glucose levels, changes or defects in any of the pathways may produce the same clinical manifestation of the disease. Genomics promises to produce the reagents and tests that will identify each sub-type making it easier to diagnose and properly treat the cause rather than the effect of diseases. The research underlying this disclosure is designed specifically for developing such tests and for developing drugs or medical foods formulated to treat the disease.

b) Environmental factors, such as diet, influence health and disease development. Chronic diseases are often influenced by one or more environmental factors acting on the genetic expression of an individual [2-5]. Diet is the most important environmental factor influencing health because of the consistent exposure to the naturally-occurring chemicals in foods. Many humans eat two to three times per day. In addition to providing for our energy needs, food contains chemicals that may cause changes in the expression of genetic information. Many food chemicals are beneficial (e.g., protein, fats, or carbohydrates) when eaten in moderate amounts but may contribute to disease development when they are overconsumed or underconsumed. In addition, certain chemicals such as minerals, vitamins, and certain dietary fats and carbohydrates are required for growth, development, and health. Optimum health, therefore, is obtained with the proper balance of the types and amounts of food chemicals.

c) The genetic makeup of each human differs causing variation in response to the same factors. Identifying healthy diets would be less complicated than it has proven to be if all humans had an identical genome. The human genome project found that although humans are 99.9% identical at the genetic level [6-8]. But the small (0.1%) differences in genomic DNA sequences produce the variation in physical appearances (height, weight, skin coloration, hair texture and color, etc) and the responses to environmental factors. These variations within genes explains why any given drug may be effective for one patient but not another (pharmacogenomics). Similarly, individuals differ in their response to the type and amount of dietary chemicals.

A well-recognized example of this interaction between a dietary chemical and genetic makeup is lactose intolerance [9-11]. Lactose in milk products can produce a painful digestive condition commonly associated with nausea, cramps, bloating, gas, and diarrhea unless the ingested lactose is metabolized by lactase, an enzyme that cleaves lactose to glucose and galactose. During most of human evolution, lactase was produced only in childhood. A mutation in the control region of the lactase gene that causes lactase to be made into adulthood occurred 10,000 to 12,000 years ago and was subsequently propagated in certain human populations. Those individuals lacking the mutation cannot tolerate milk, a selective disadvantage, as milk is highly nutritious. This easily understood yet dramatic example is a model for the more subtle metabolic differences that occur with variations in other genes and exposures to other types of chemicals. Weight, for example, can be control a lucky collection of normal gene variants that metabolize nutrients and avoiding the types and amounts of foods that contribute to weight gain.

The Present Solution

Four broad approaches are used to identify gene or protein targets for developing diagnostics and drugs used to treat diet-associated diseases: (a) identification of genes, ESTs, transcripts or proteins that differ in presence, quantity or expression between normal and diseased tissues, (b) identification of clinical factors, e.g., cholesterol levels, that correlate with disease, and concomitant identification of genes involved in the pathway producing those factors, e.g., HMG CoA reductase), (c) epidemiological research associating an environmental factor with incidence or severity of disease and identification of a gene or protein associated with that disease and/or environmental factor, or (d) identification of chromosomal regions associated with disease and choice of most likely candidate genes within those regions.

a) Identification of DNA/RNA/protein differences between normal and disease tissues: Molecular comparisons identify genes or transcripts or proteins whose levels are different among diseased vs. normal tissues [12]. Oligonucloetide array technologies provide a means to rapidly screen for the expression profiles of thousands of genes at one time. The genes identified as being differentially expressed between diseased and healthy tissues may be developed into diagnostics or drug targets. The risks of relying only upon differences in gene or protein expression to develop diagnostics or drugs are that:

(i) such differences do not necessarily discriminate between differences that are either a cause or an effect of a disease state.

(ii) the same disease may be produced by different pathways—a single marker or target will rarely identify or be effective against all forms of the disease.

(iii) genetic differences confound analyses because any given gene could be normally, but differently expressed among individuals.

(iv) environmental factors, which are rarely controlled in studies of isolated human tissue, might also cause differences in expression.

b) Identification of candidate genes from associations with measured clinical factors (e.g., cholesterol levels) that correlate with disease: Cholesterol levels that are 1.2× the recommended level of 200 mg/dl have a 2 fold greater risk of heart disease (American Heart Association: (http://www.americanheart.org/presenter.jhtml?identifier=4500). Researchers therefore targeted the control point of the cholesterol biosynthetic pathway for development of drugs (statins) to control the level of total serum cholesterol. Statins do reduce cholesterol levels and reduce the relative risk of coronary heart disease by ˜25%. However, the absolute reduction in risk in one study (as an example) was 8.3% in the placebo group as compared to 6.4% in the pravastatin treated group [13] other references are found in ACC/AHA/NHLBI Clinical Advisory on the Use and Safety of Statins, found at: http://www.nhlbi.nih.gov/guidelines/cholesterol/statins.htm).

Similar strategies of identifying known pathways, known genes, and known enzymes as drug targets are used for almost all major diseases including Alzheimer's, cancer, diabetes, and obesity.

The major drawbacks of this strategy are that:

(1) controlling a single aberrant physiological factor (such as total cholesterol) does not affect the many other pathways that contribute to the development of the disease.

(2) genetic differences among individuals are not considered.

(3) environmental influences of diet is also not considered.

c) Identification of candidate genes the expression of which is correlated with environmental factors linked to a disease state by epidemiological evidence: Another strategy for identifying candidate genes is to analyze clinical data to make an epidemiological association between a gene, and environmental factor, and a disease [2]. For example, HMG CoA reductase, cholesterol, and heart disease.

d) Identification of chromosomal regions associated with disease and choice of most likely candidate genes within those regions: Genetic methods for identifying quantitative trait loci (QTL—A genetic region known to effect phenotypic variation in continuously varying traits like skin color, weight, etc.) have been developed over the past 15 years. Such methods identify regions of chromosomes encoding one or more genes that contribute to the development of a complex disease, e.g., diabetes [14-16]. Insulin-dependent diabetes mellitus (IDDM) is an example of the power and limitations of this approach. There are about 15 QTL loci, (IDDM1 through NIDDM15) regulating insulin, glucose, lipid, or hormone levels that directly contribute to diabetes development. Approximately 1700 OTLs for diabetes, obesity, cancer, and other conditions have been determined in laboratory animals (http://www.informaticsjax.org/searches/marker_report.cgi) and a smaller subset has been identified in humans.

Although QTL methodology provides valuable information, identifying all the candidate genes within QTLs is difficult because:

(i) QTLs are specific only for the population (or laboratory animal strains) analyzed.

(ii) The size of the region identified is variable (˜1 to 20 cM, equivalent to 1-20 million base pairs) and may encode many genes of known or unknown function.

(iii) A QTL may be specific to environmental conditions under which they were determined [17, 18] and,

(iv) The contribution of a given locus to a disease process varies between ˜1 and ˜25% [19], and individual effects may be too small to accurately be measured with existing biological techniques [16]

The limitations of QTL mapping may be circumvented using positional mapping and cloning in selected human populations or families where the chronic disease develops earlier than normal. Myriad Genetics Corporation has identified two breast cancer genes, BRCA1 and BRCA2, in early onset breast cancer patients using positional cloning and sequencing methods. More recently, 5 loci (MODY1 through MODY5) causing the autosomal dominant disease, mature onset diabetes of the young (MODY), have been discovered by positional cloning and characterized by DNA sequence analyses (rev. in [20]). MODY accounts for between 2 and 5% of Type II diabetes. The genes at these loci, hepatocyte nuclear factor 1 alpha (HNF-1 alpha), glucokinase, HNF-4 alpha, HNF-1 beta, and insulin promoter factor 1 have yet to be linked to non-MODY diabetes development [21].

Approximately 20 candidate genes selected for their likely role in diabetes or from various NIDDM and obesity QTLs (some of these loci overlap) have been analyzed in genetically diverse populations with high incidence of diabetes (Table 1).

TABLE 1
Possible Candidate Genes for Type 2 Diabetes^{a}

| Mutated | | | |
|---|---|---|---|
| Gene | Function | Effect | Linked to |
| HNF-4α, | Transcription Factors | ↓Insulin Secretion | MODY (maturity-onset |
| HNF-1β | | | diabetes in the young) |
| IPF-1, | | | human |
| NeuroD1 | | | |
| HNF-1α | Transcription Factor | ↓Insulin Secretion | MODY Oji-Cree diabetes |
| Glucokinase | Glucose Metabolism | ↓Insulin Secretion | MODY |
| Calpain-10 | Protease | Unknown | Diabetes 2 in Mexican and |
| | | | African Americans |
| PPARγ | Transcription Factor | ↓Insulin Sensitivity | Diabetes 2 |
| Insulin | Transmits Insulin Signals | ↓Insulin Secretion | Human diabetes (rare) |
| Receptor | into cells | and Sensitivity | mouse model |
| IRS1 and -2 | Insulin Signaling | ↓Insulin Sensitivity | Mouse Models |
| Akt2 | Insulin Signaling | ↓Insulin Sensitivity | Mouse Models |
| 11-β-HSD | Glucocorticoid Synthesis | ↑Blood Lipid, | Mouse Models |
| | | ↓Insulin Secretion | |
| UCP2 | ↓ATP Synthesis | ↓Insulin Secretion | Mouse Models |
| Resistin | Fat Cell “hormone” | ↓Insulin Sensitivity | Mouse Studies |
| Adiponectin | Fat Cell “hormone” | ↑Insulin Sensitivity | Mouse, Human Studies |

^{a}Adapted from: [22]

As an example, Bell and his associates [21] found a candidate gene at an NIDDM QLT that appears to be involved in the development of Type II diabetes. They carried out a genome-wide screen for Type II candidate genes in Mexican American families and identified a locus named NIDDM1. Subsequent positional cloning showed that Calpain 10, a cysteine protease, within the NIDDM1 locus was associated with Type II diabetes in individuals from Botnia, a region of Finland, and in Mexican-Americans. Positional cloning to identify genes within QTLs is possible when the gene contributes significantly to the disease process, as is the case with Calpain 10. Identifying the genes or alleles within the other QTL for diabetes or other chronic diseases will require more sophisticated strategies.

Although alleles of some of the genes show associations with disease development, most show no associations in the populations tested. Such results may be due to the small number of individuals analyzed and the need to analyze multiple alleles and genes with multiple phenotypic characterizations—most studies use single variant analyses. Hence, the identity of the candidate genes in NIDDM and obesity QTLs have not been verified.

Although a powerful technique, identifying all genes contributing to chronic disease development by QTL analyses by itself is prohibitively expensive because of the very large population needed to achieve reliable QLT identification, meaning that many QTL studies are prone to identifying the wrong genes as being associated with a trait, they may miss important loci and genes, and they inherently have problems with resolution, due to the size or the QTL fragments.

Problems Inherent in the Present Solutions

Many of the specific problems have been discussed above. The consequences from the current research and pharmaceutical approach of targeting a single gene, protein, or enzyme for drug development (assuming that: each patient is genetically identical, that a given disease has one physiological defect, and that environmental factors are negligible or unimportant) are that chronic diagnosis, is imprecise and treatments of disease are often ineffective. Health care costs, from insurance through delivery, are increasing exponentially while the quality of life in Western societies is static at best. Genomic research in academia and the pharmaceutical industry promises to alleviate severity of chronic diseases through the development of better diagnostics and medicines. However, the strategies and drug development pipelines fail to consider or address the interplay of environmental factors and genotype. The consequences are:

Medicines that, on average, are effective in only 6 of 10 individuals.

Trial and error approaches to treating chronic diseases increase health care costs.

Increased incidence severity of chronic diseases and resulting personal and societal costs.

### BRIEF DESCRIPTION OF THE INVENTION

A method for identifying diet-regulated disease-associated polynucleotides by comparing gene expression between two inbred genotypes (strains) in response to different diets and identifying those differentially expressed polynucleotides that overlap with independently-derived diet-regulated QTLs. Methods are also disclosed for use in disease screening, monitoring and treatment, and for formulating medical foods. Arrays are also disclosed. Various newly identified diet-regulated disease-associated genes and ESTs are also disclosed, as are compositions of medical foods and dietary supplements.

### BRIEF DESCRIPTION OF THE DRAWINGS

FIG. 1: Genes Regulated by Diet. Previously unknown disease/diet associations for these genes are shown.

FIG. 2: Genes Regulated by Diet Depending Upon Genotype. This table shows previously undisclosed data.

FIG. 3: Genes regulated by diet, genotype, or genotype-diet interactions mapping to QTLs. This table shows previously undisclosed data.

FIG. 4: Diet-regulated Genes and Disease or Complex Phenotype Processes (alphabetical by function). This table shows data known at the time of filing.

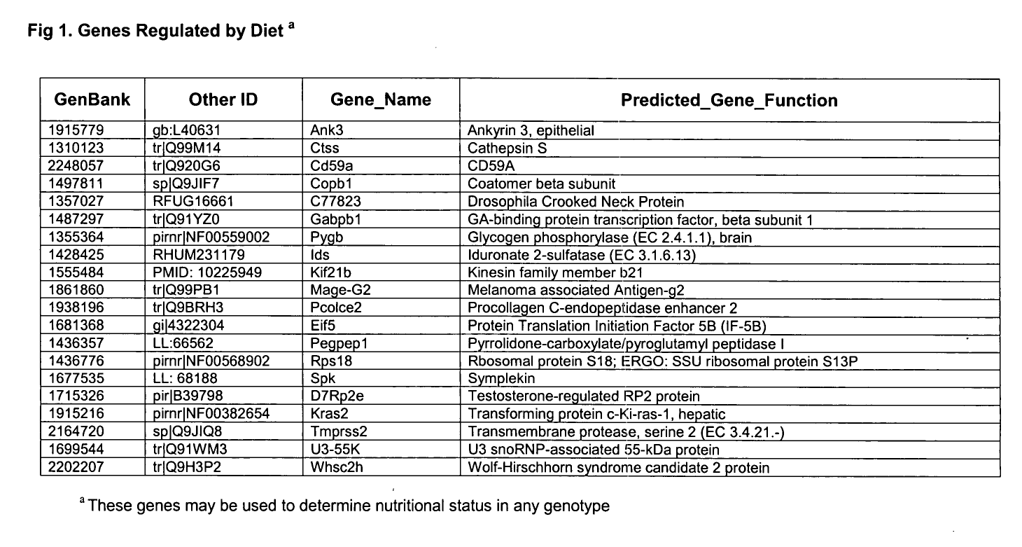

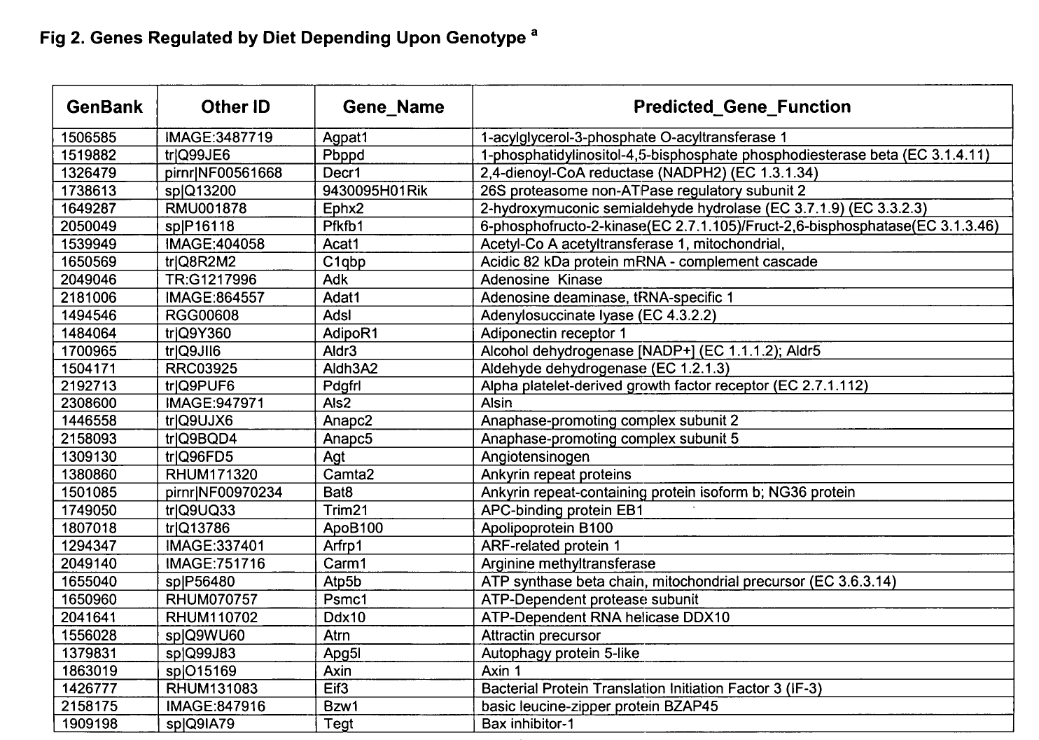

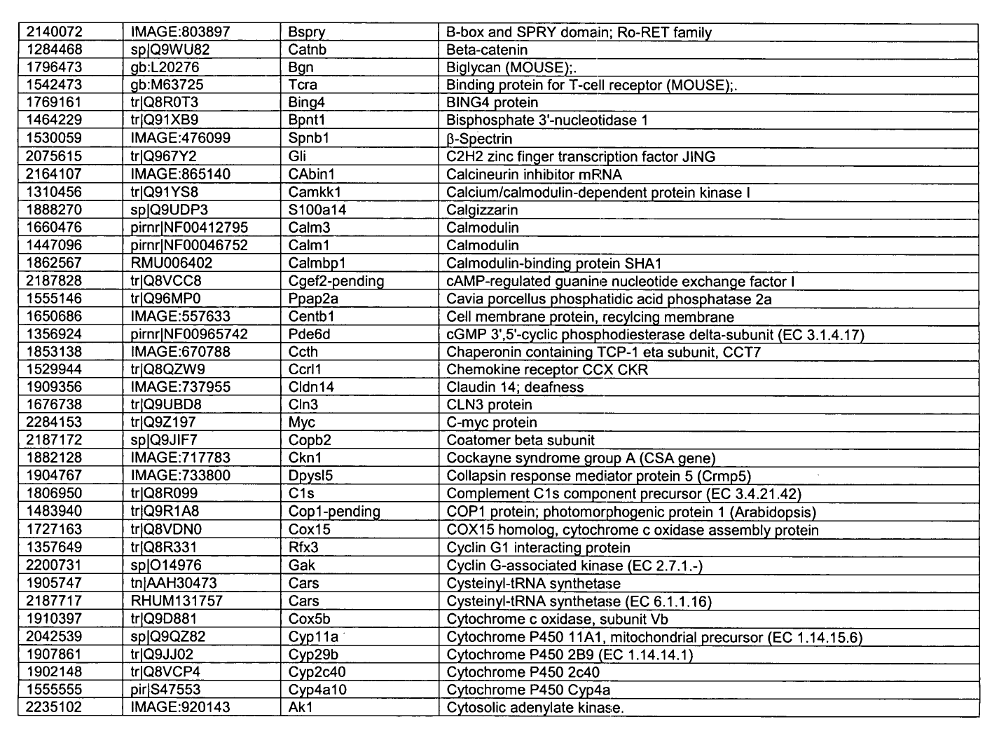

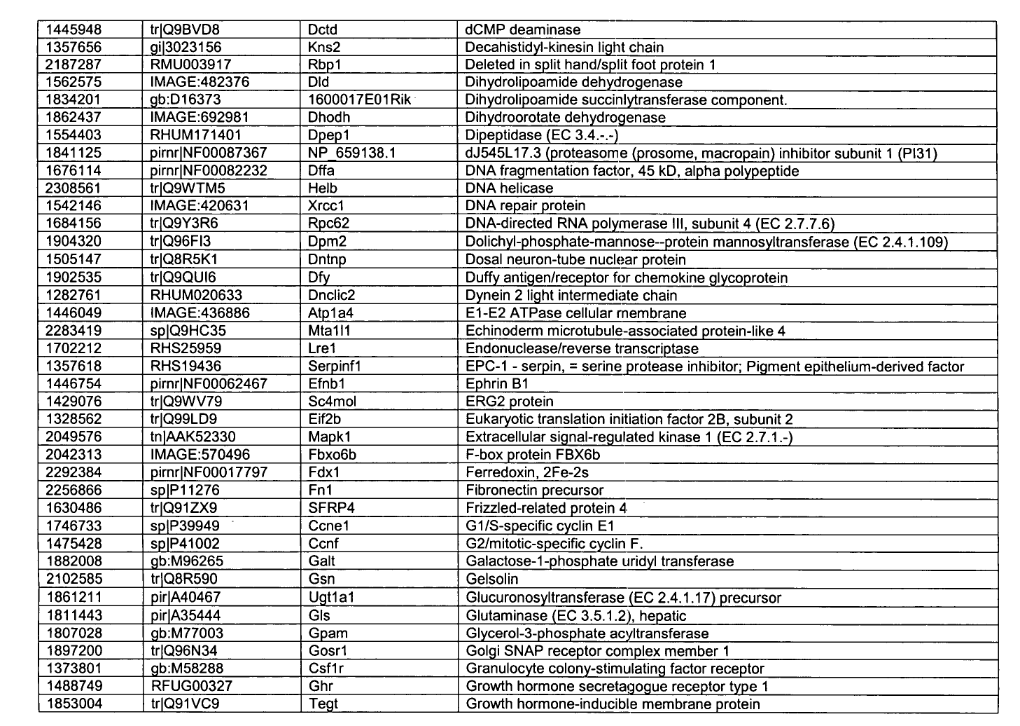

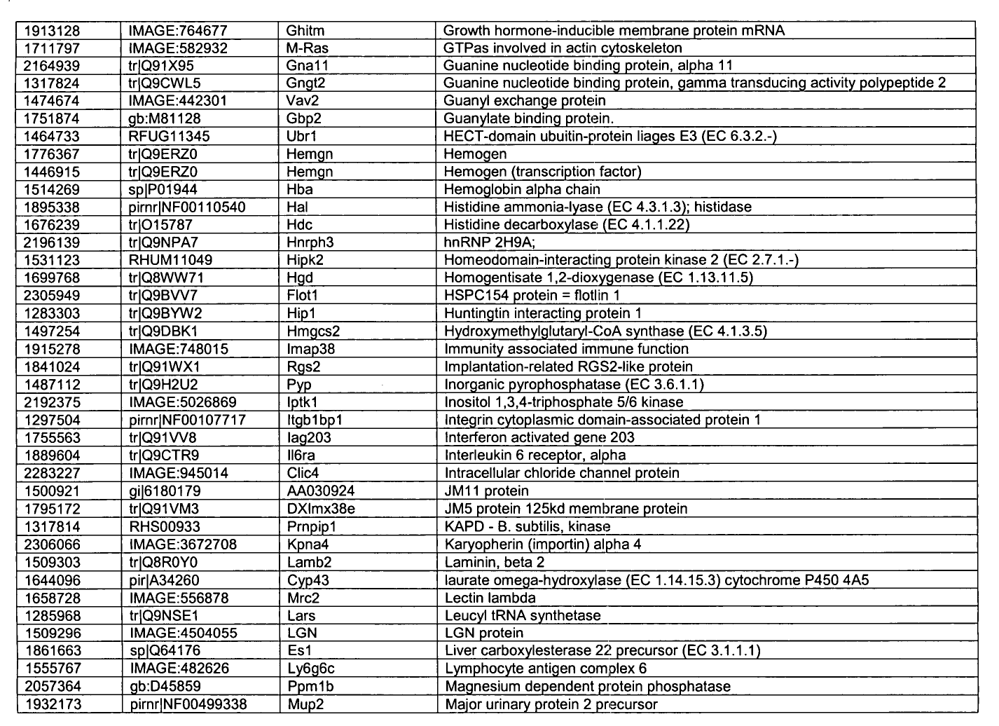

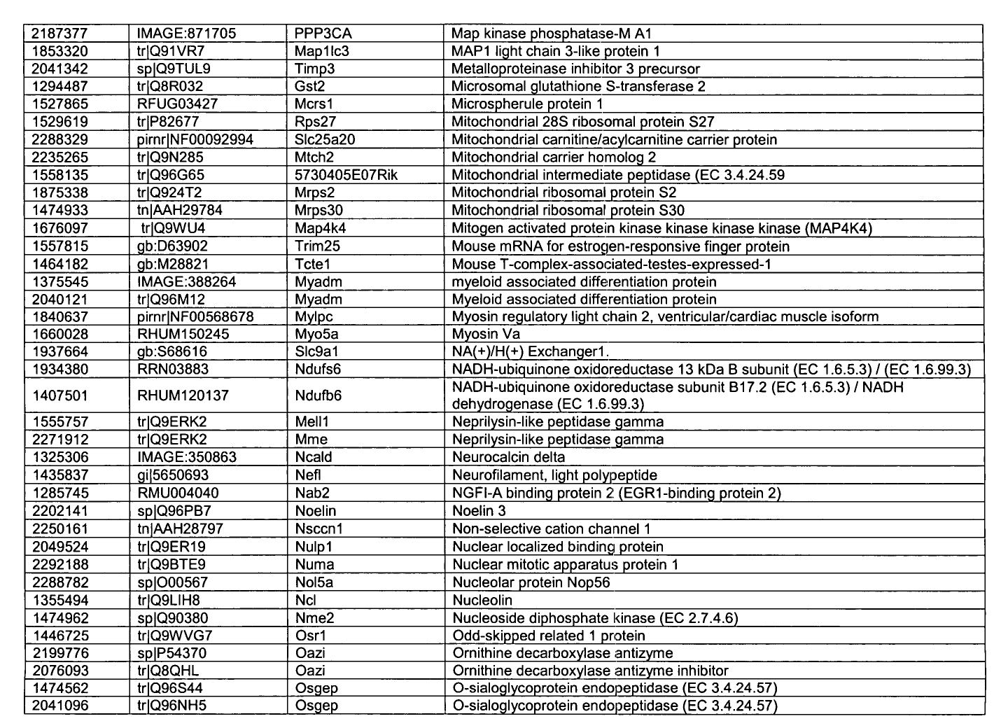

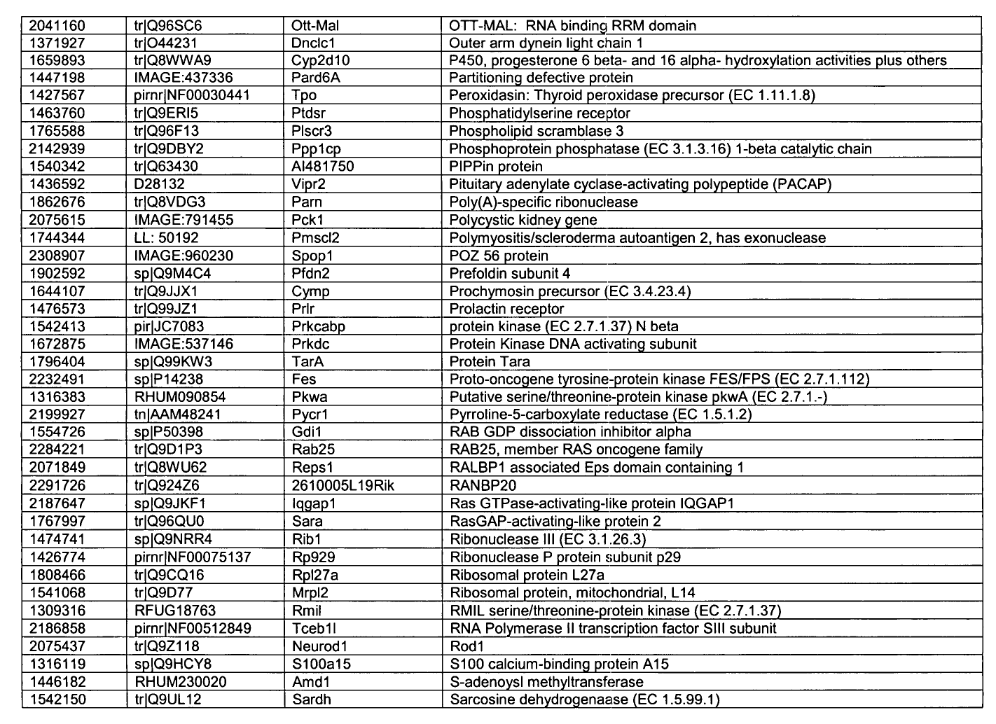

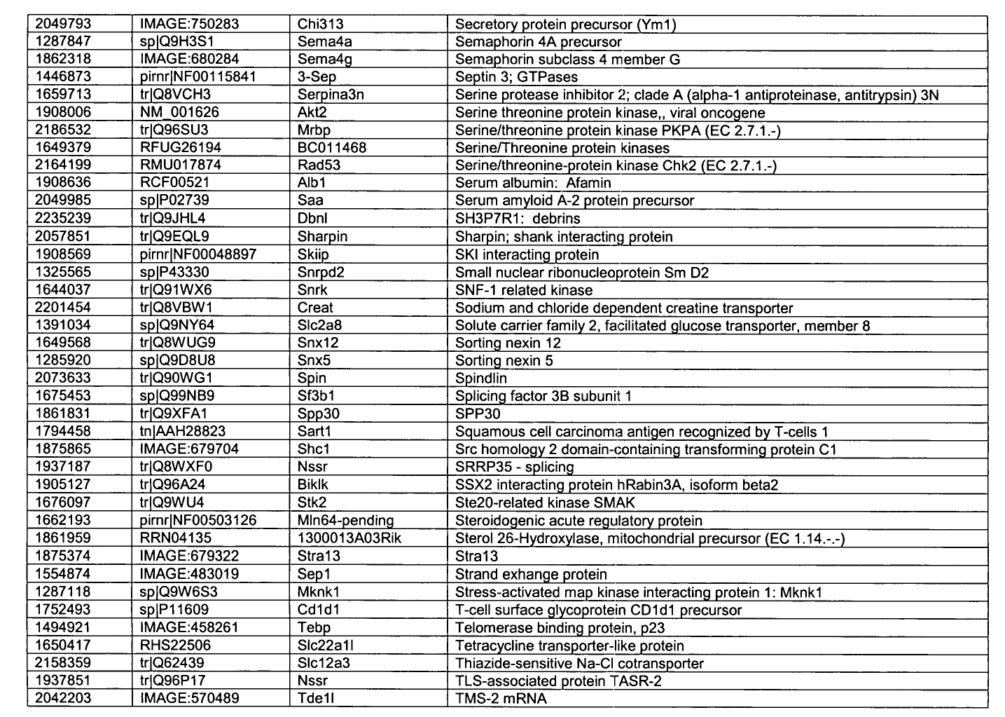

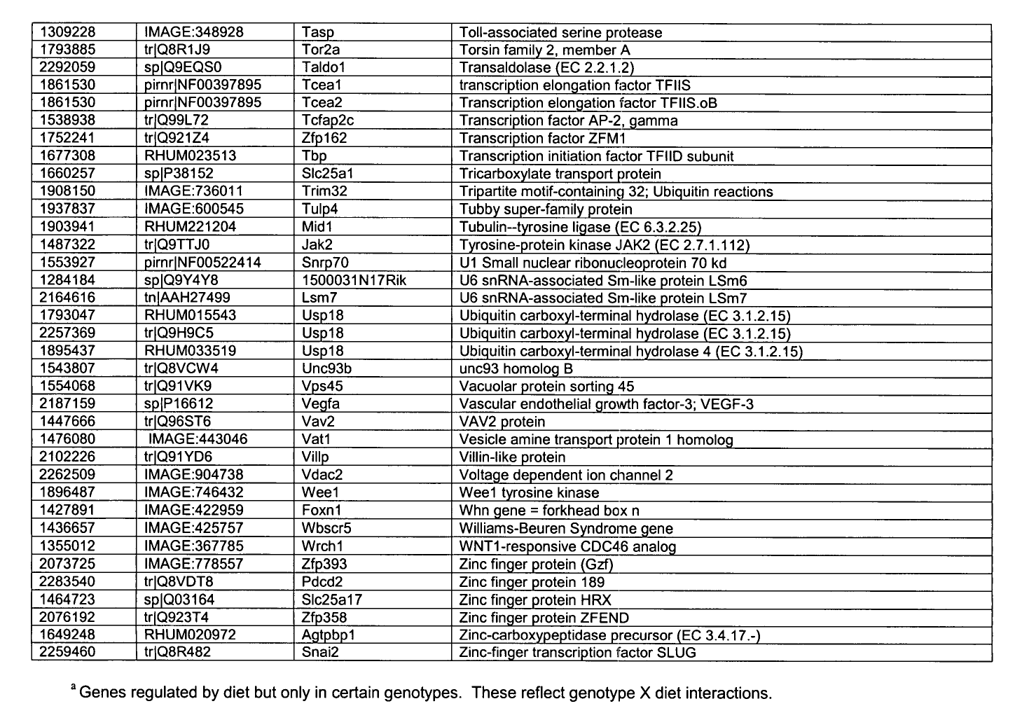

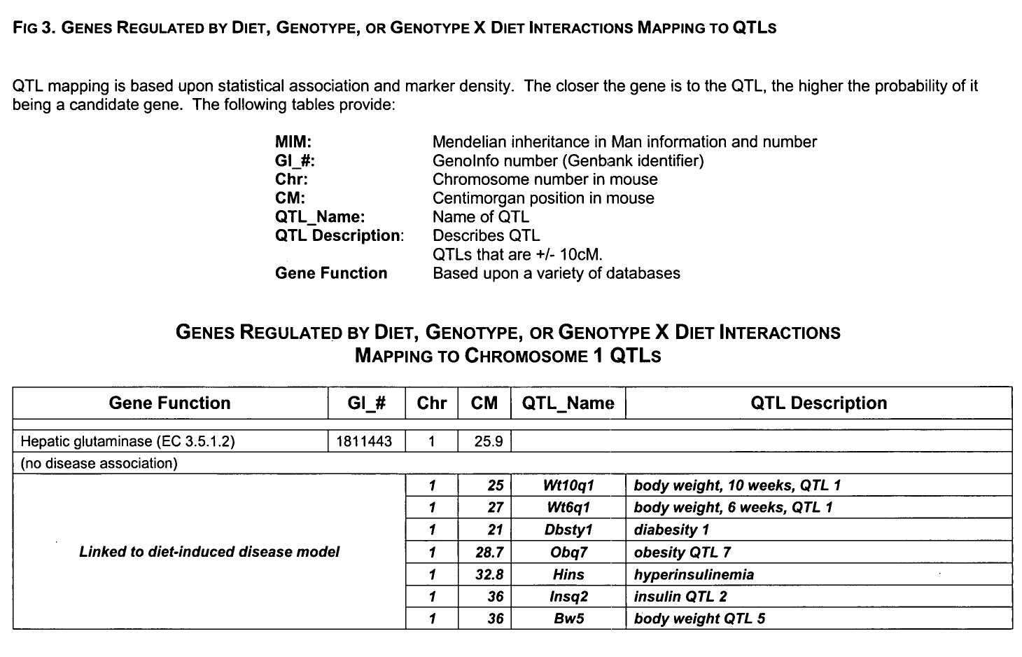

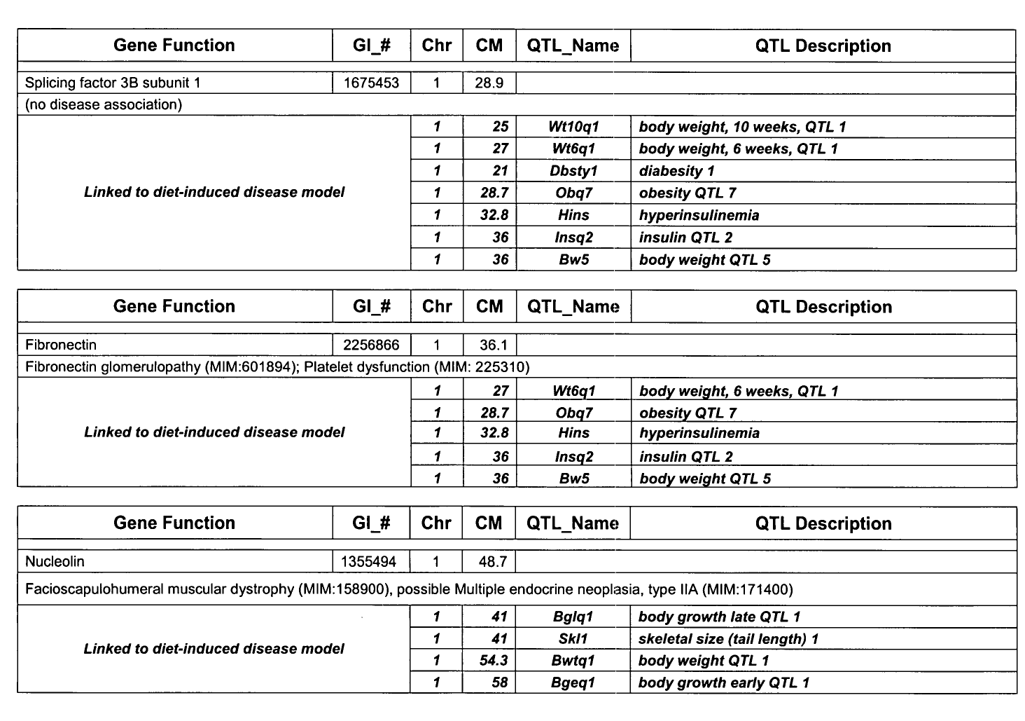

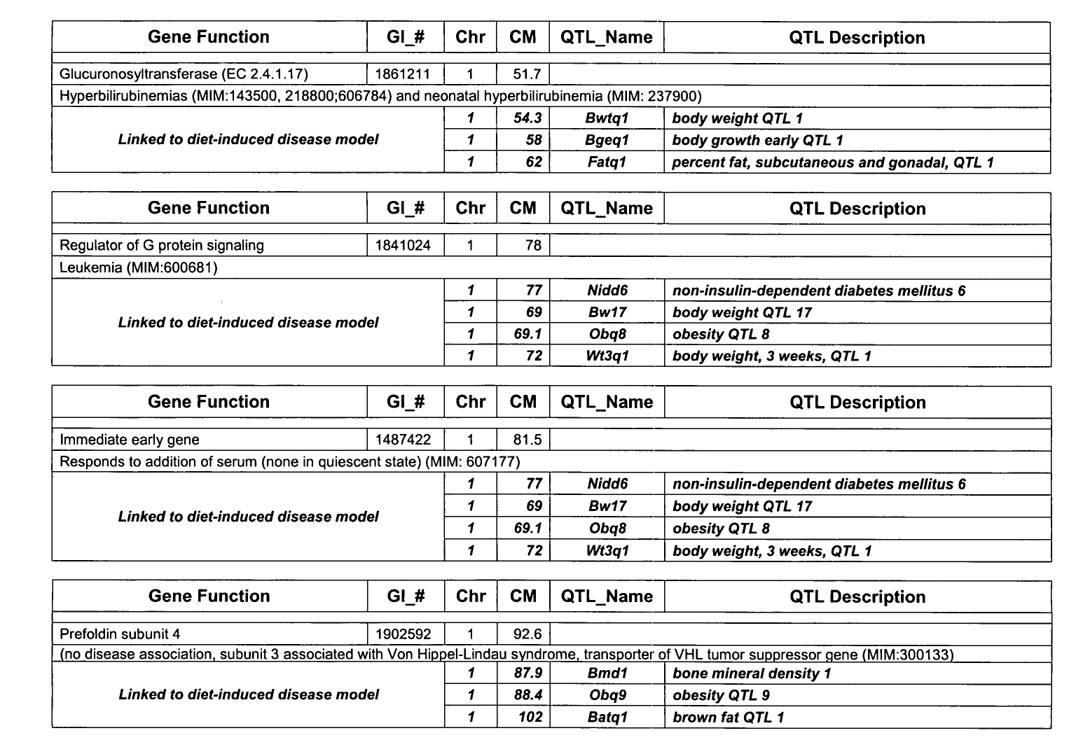

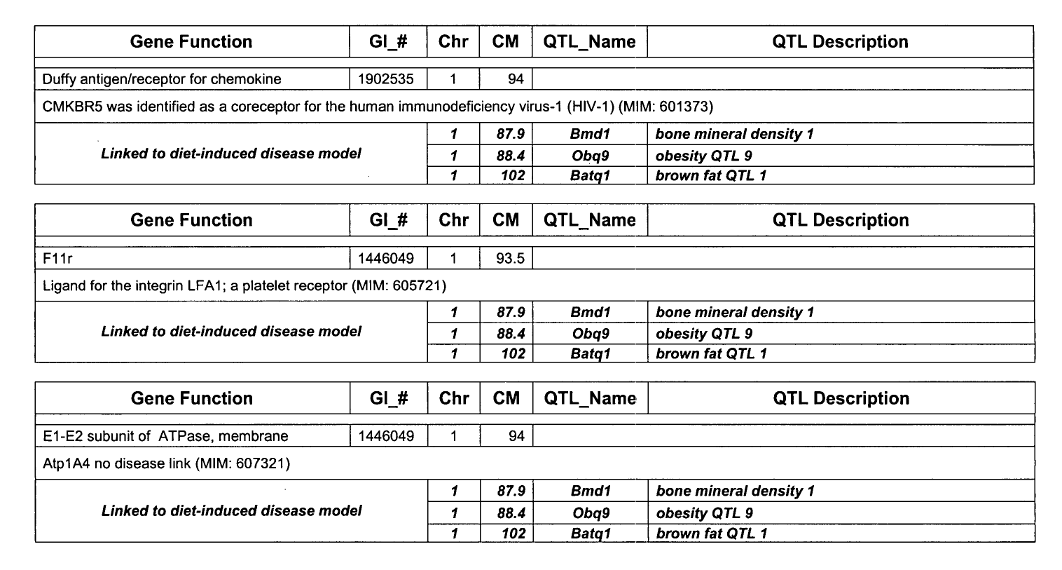

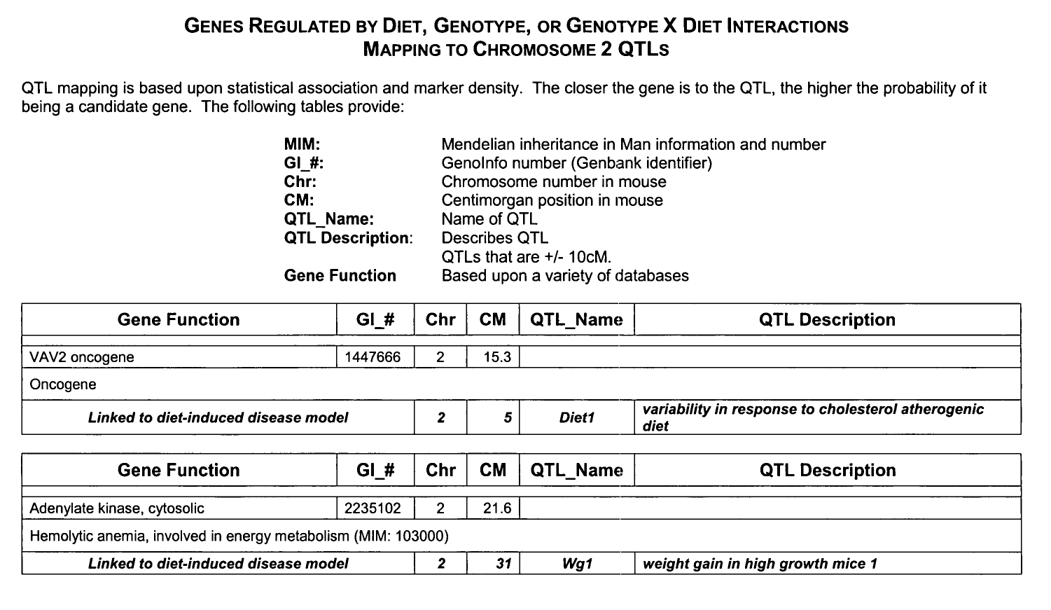

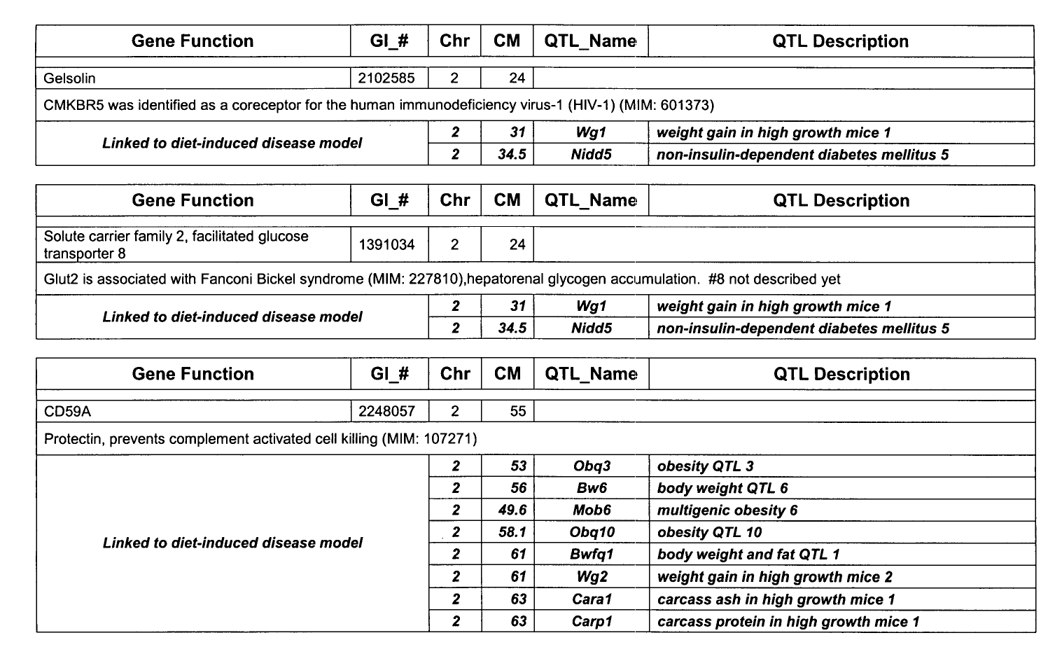

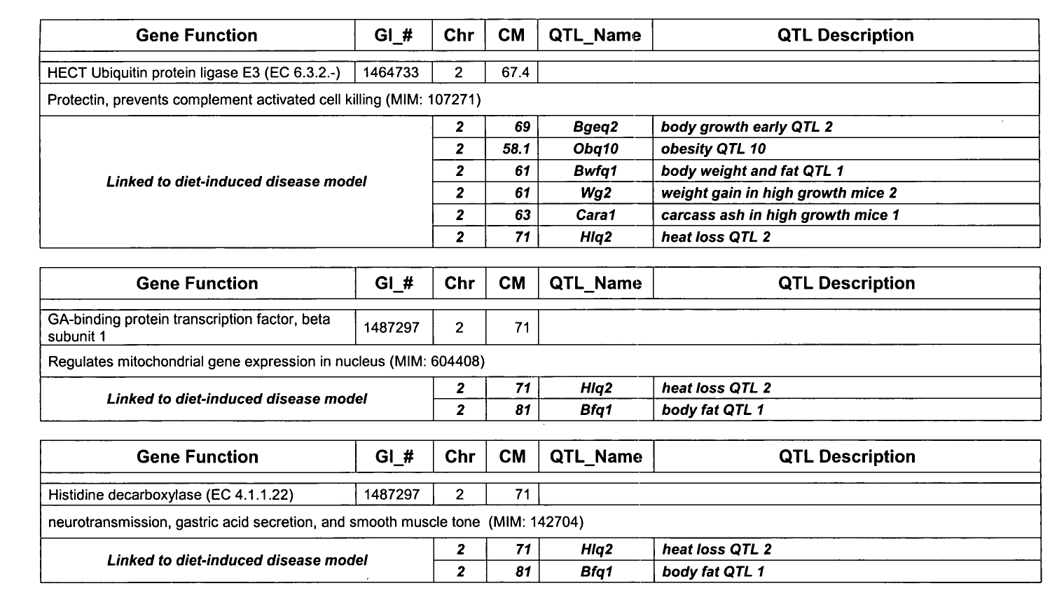

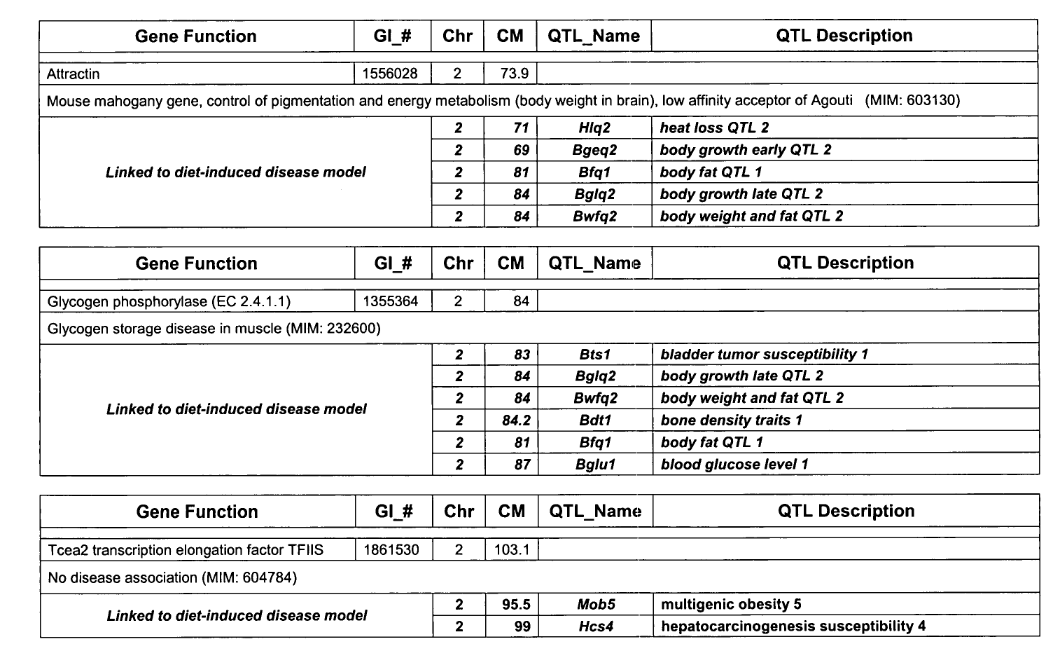

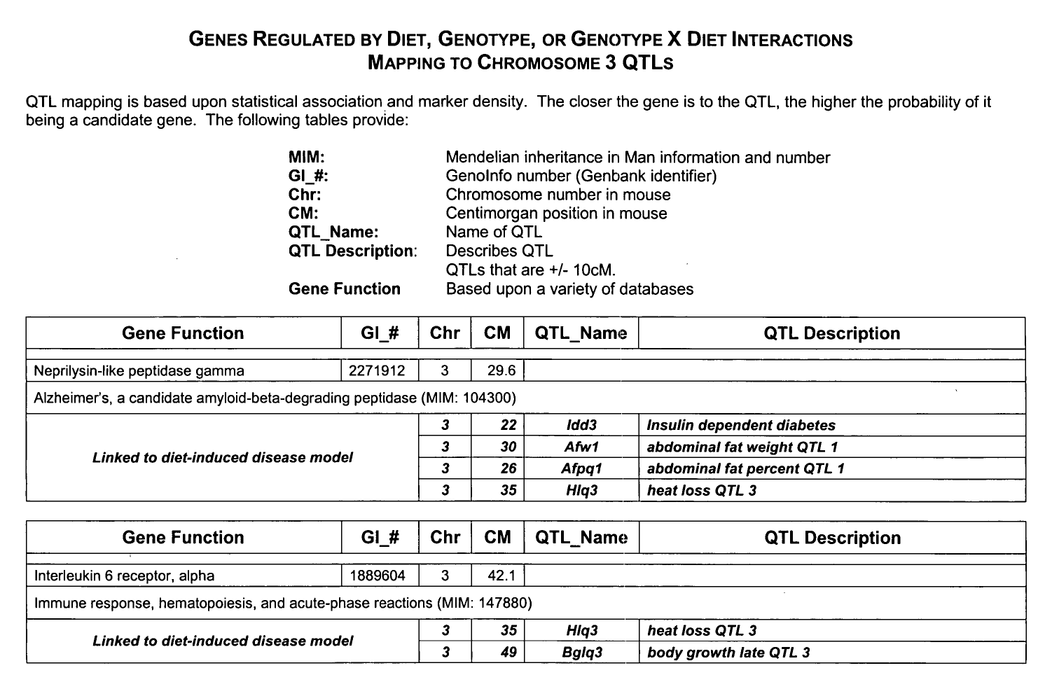

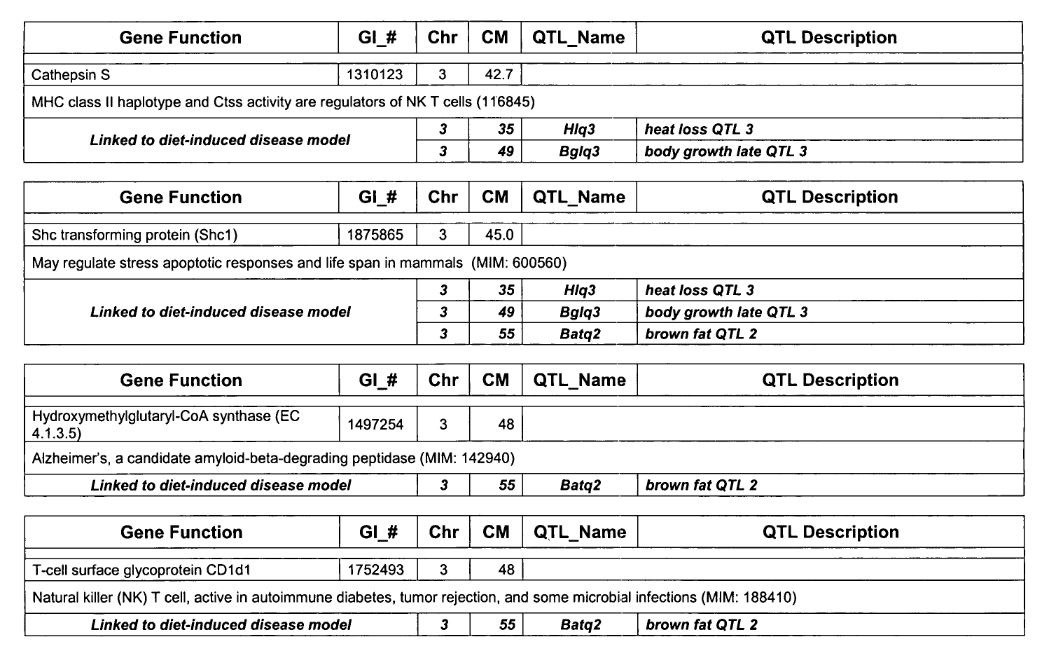

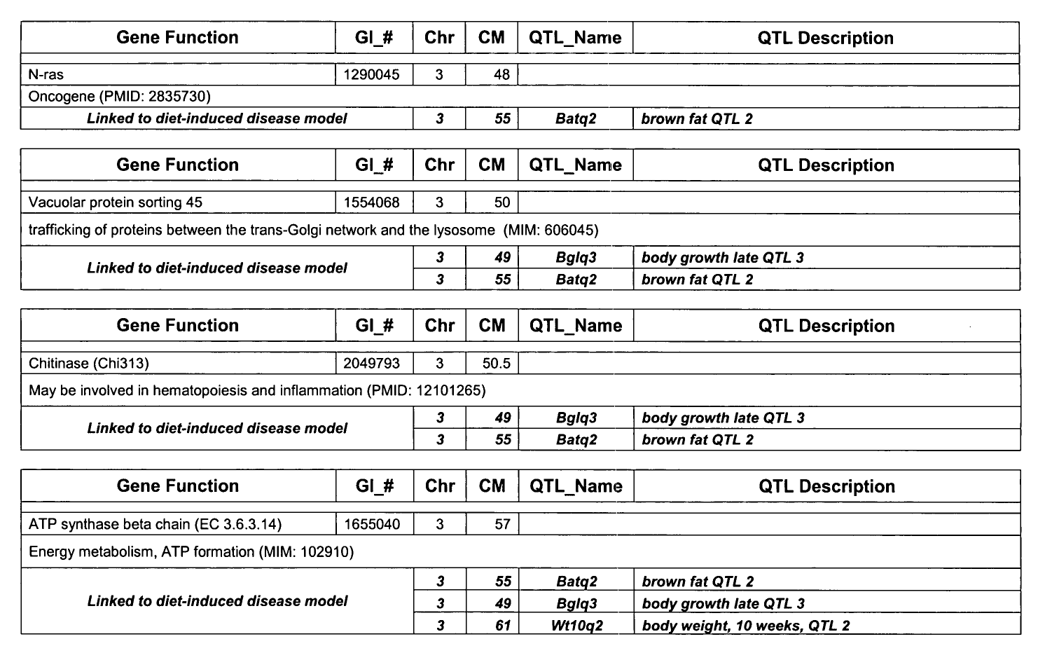

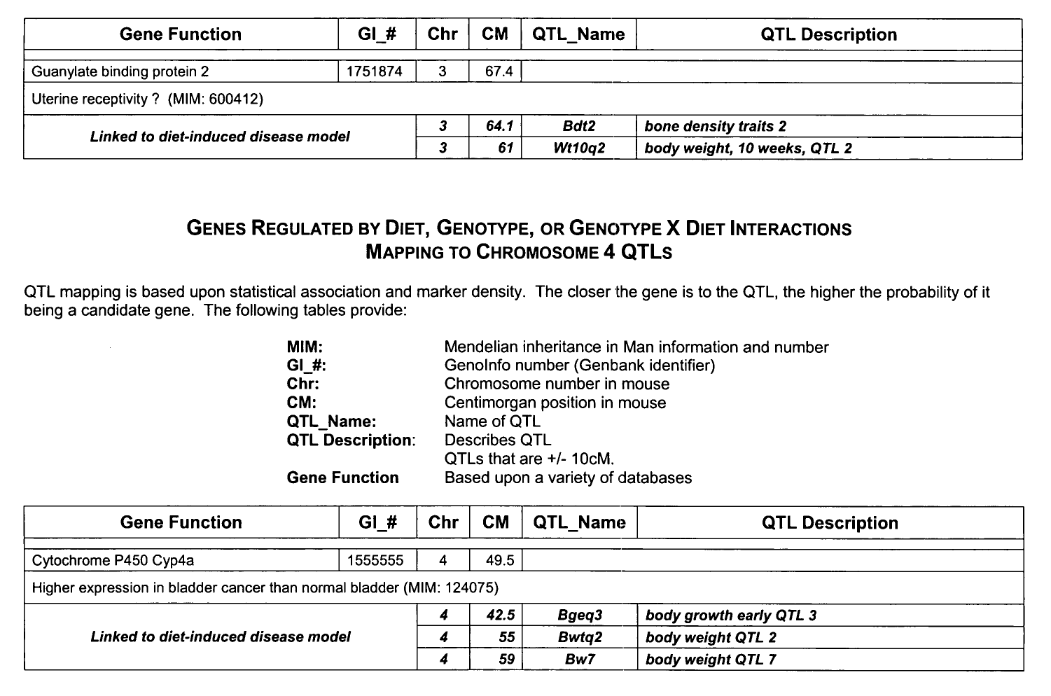

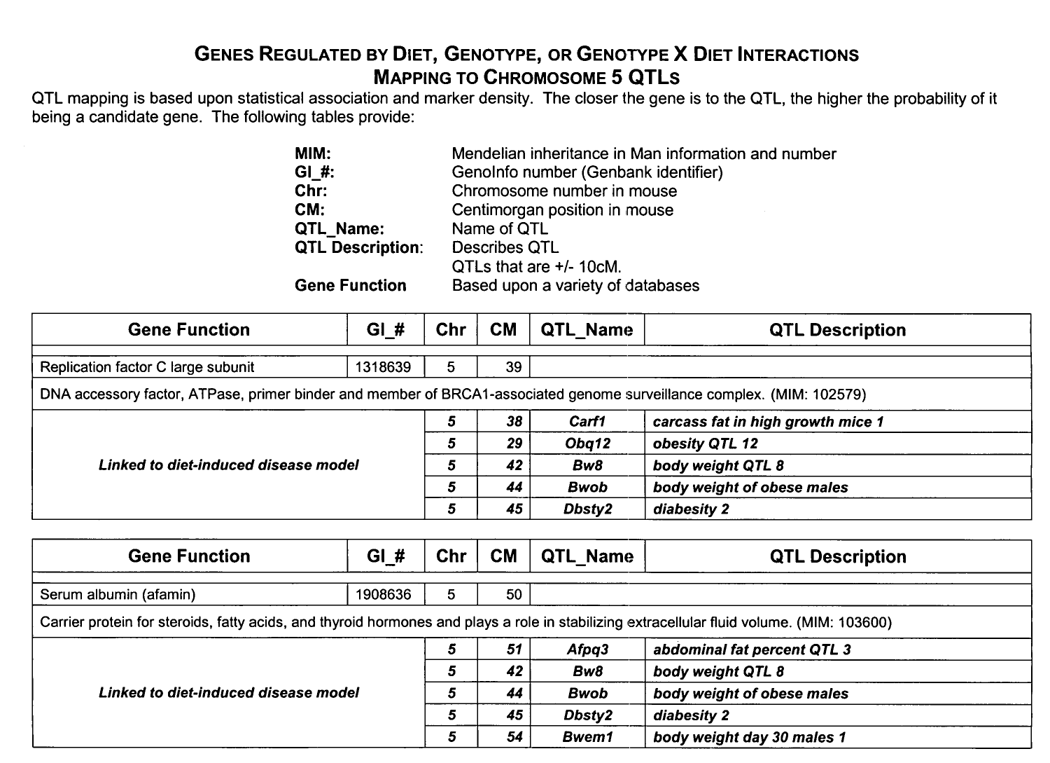

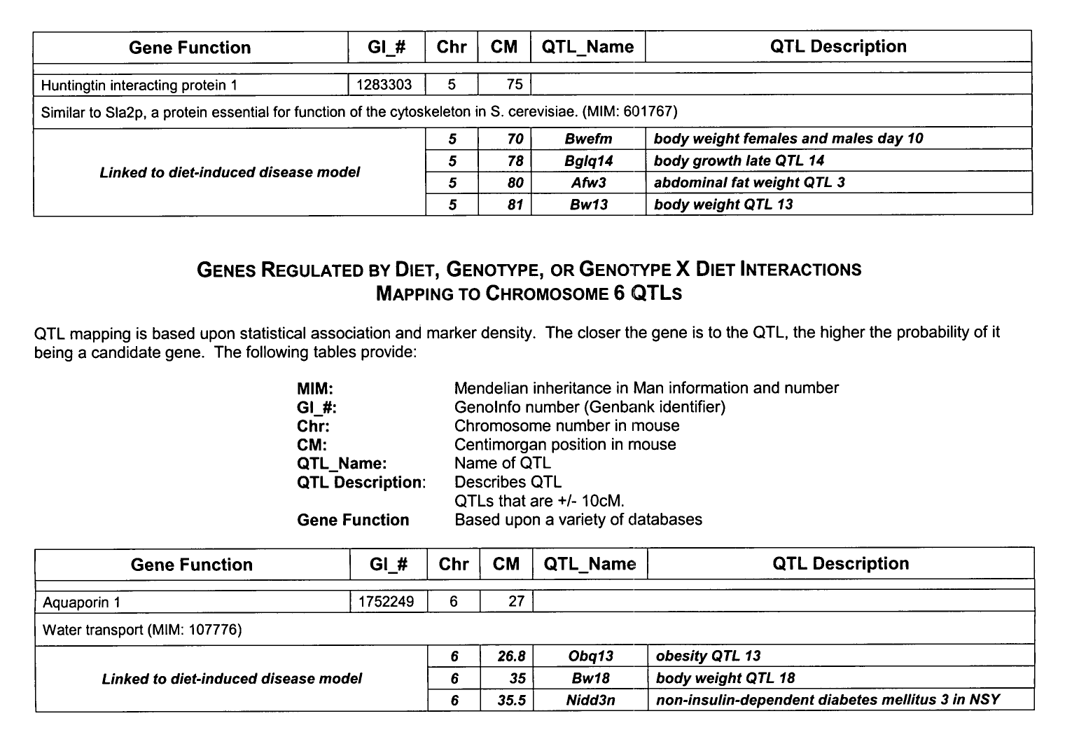

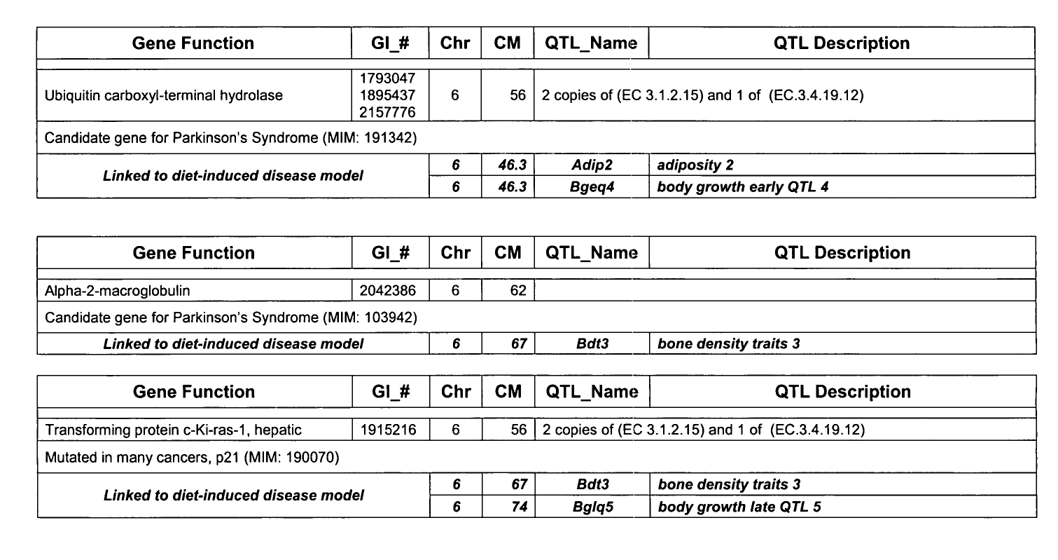

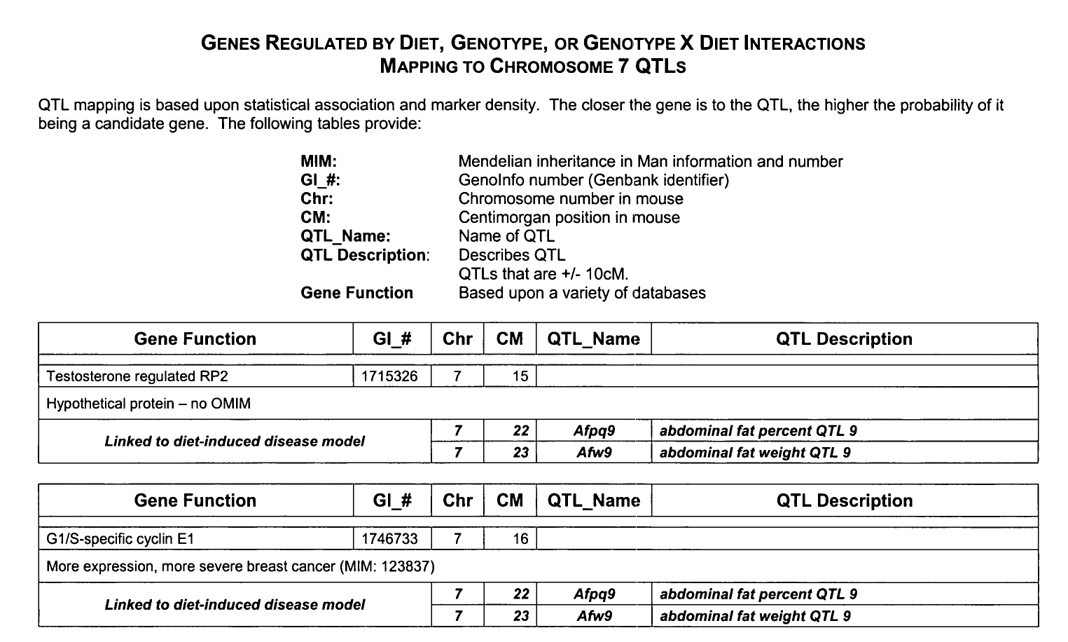

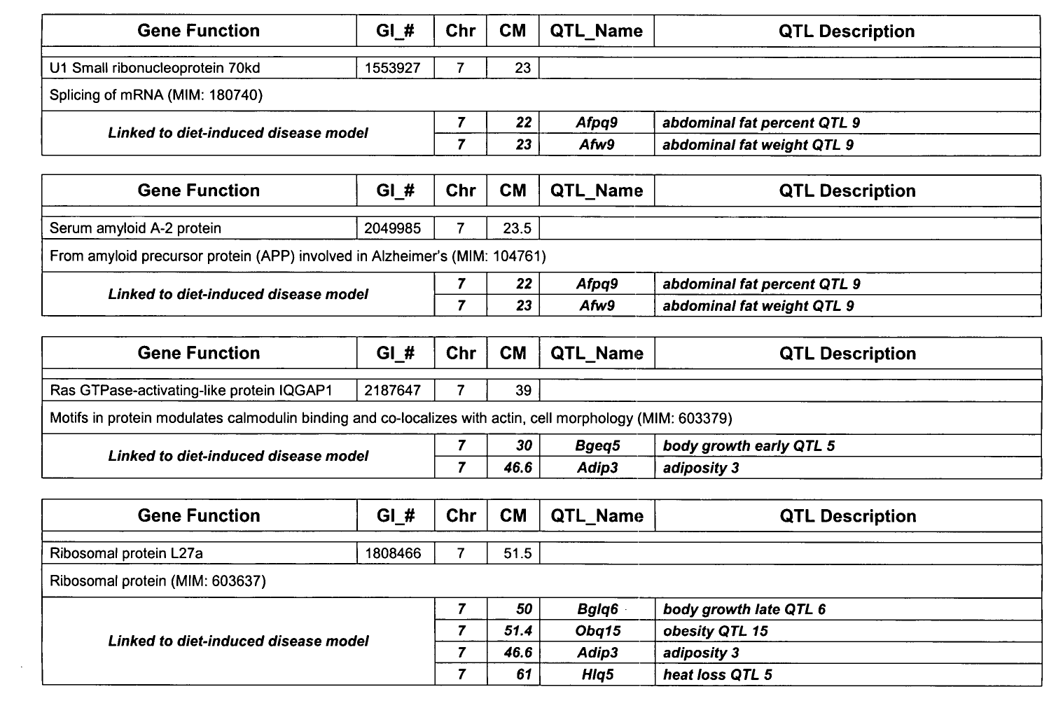

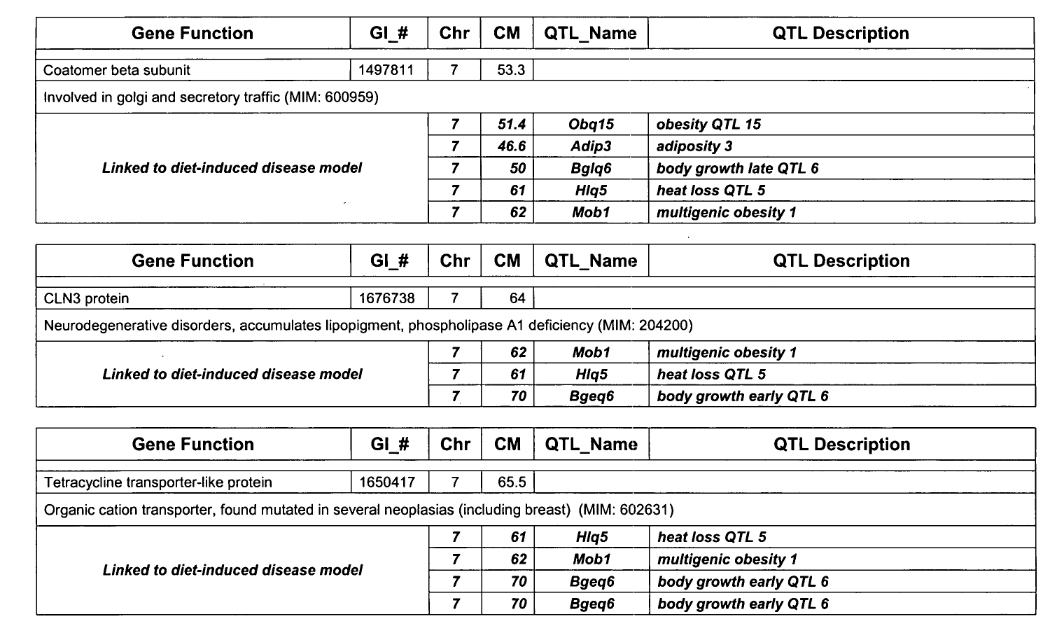

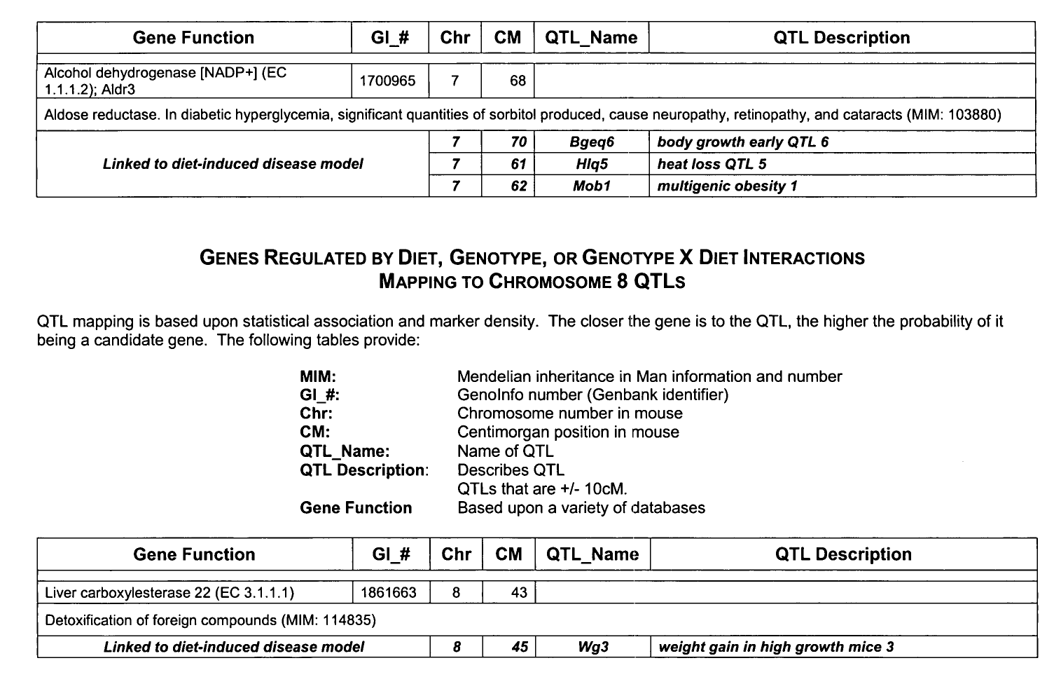

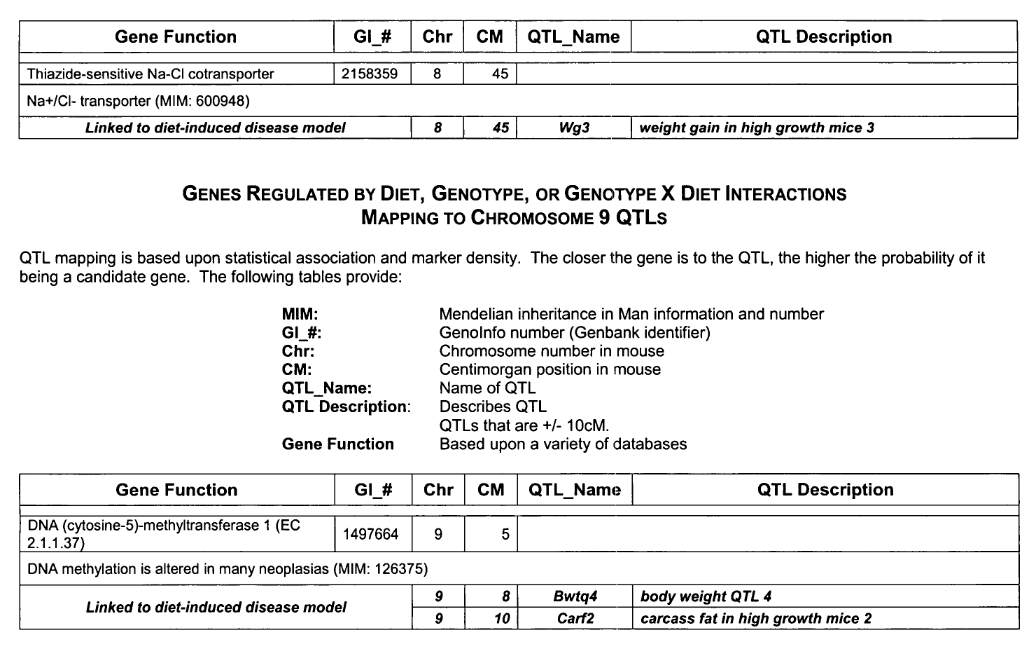

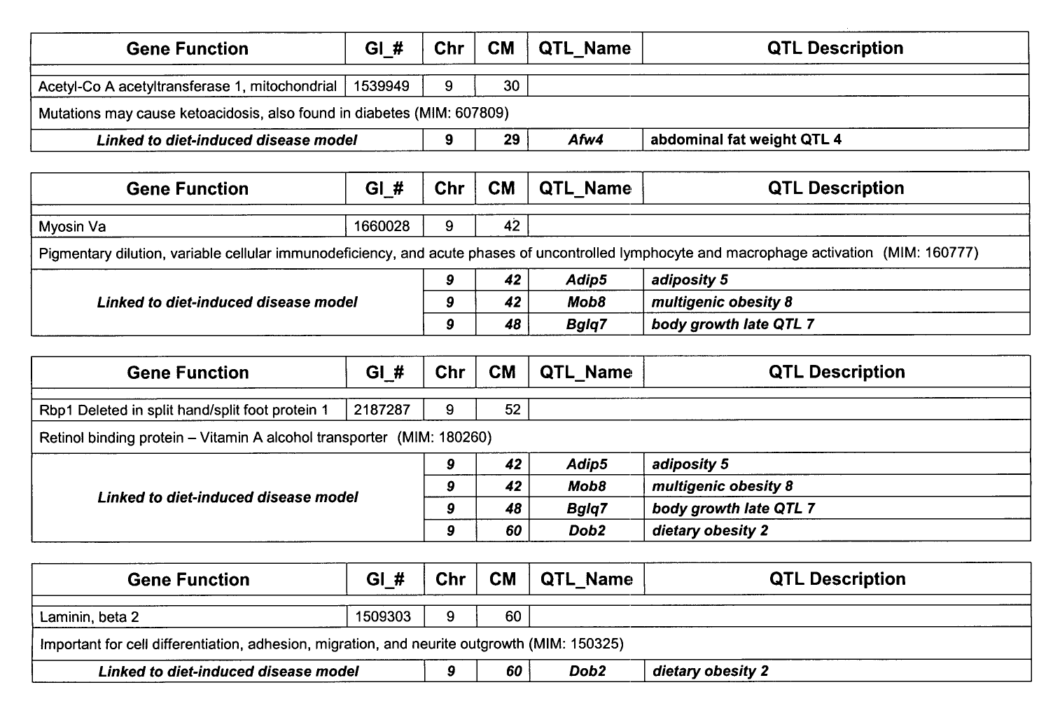

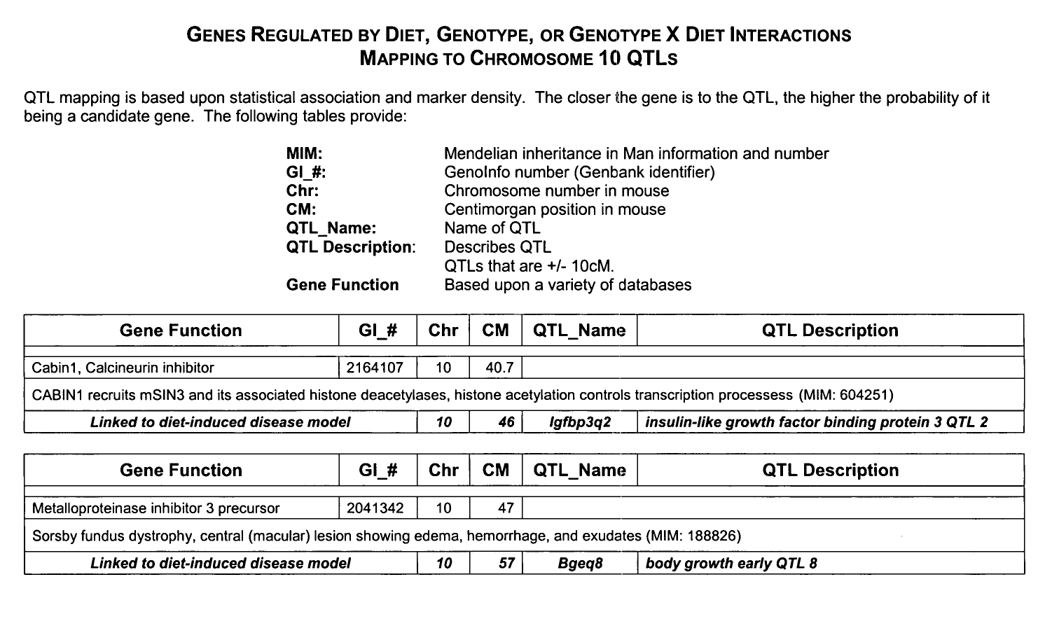

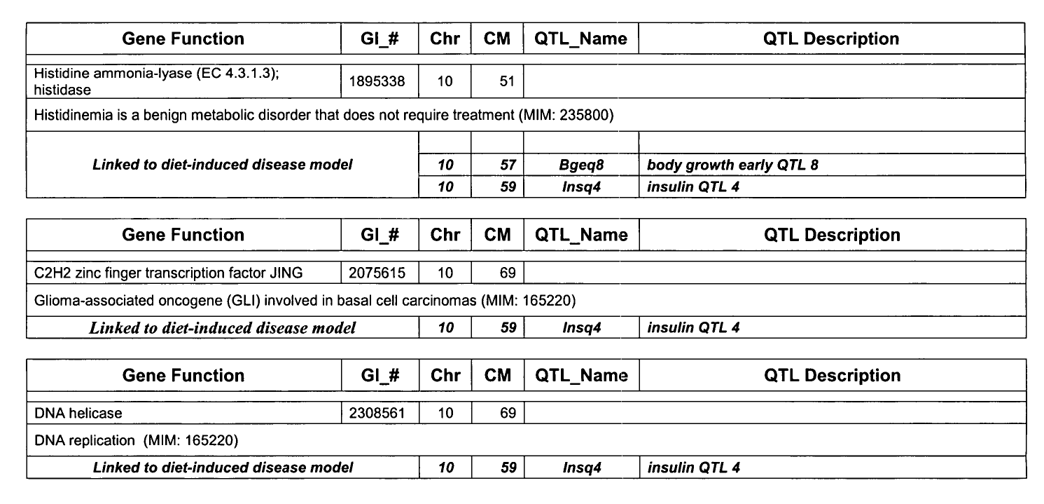

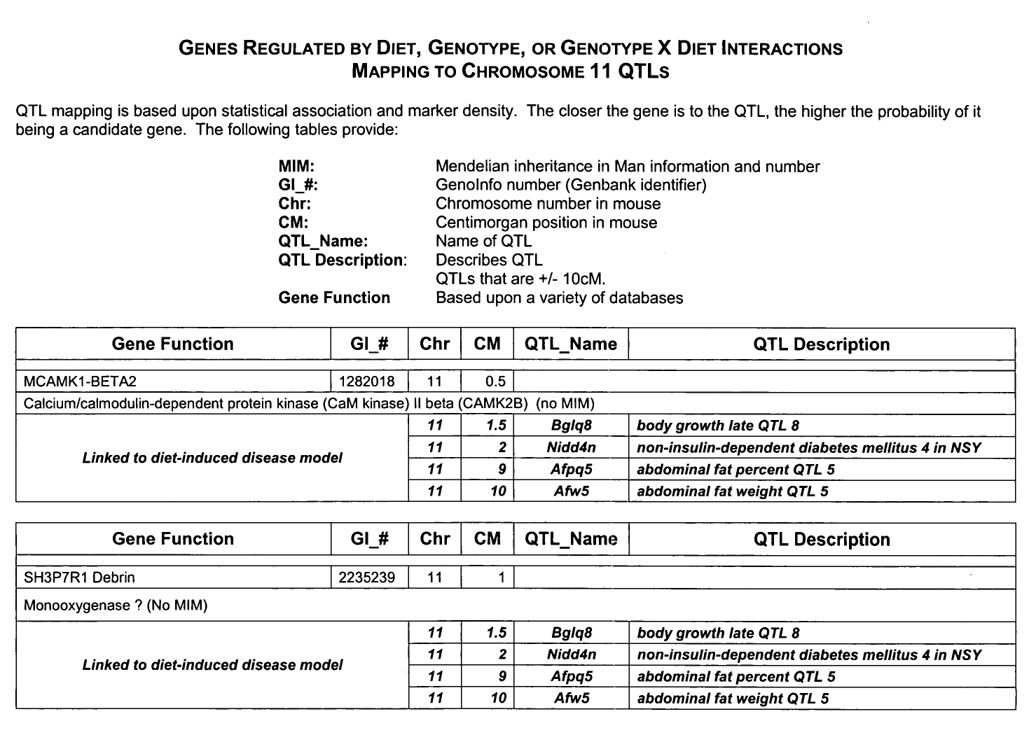

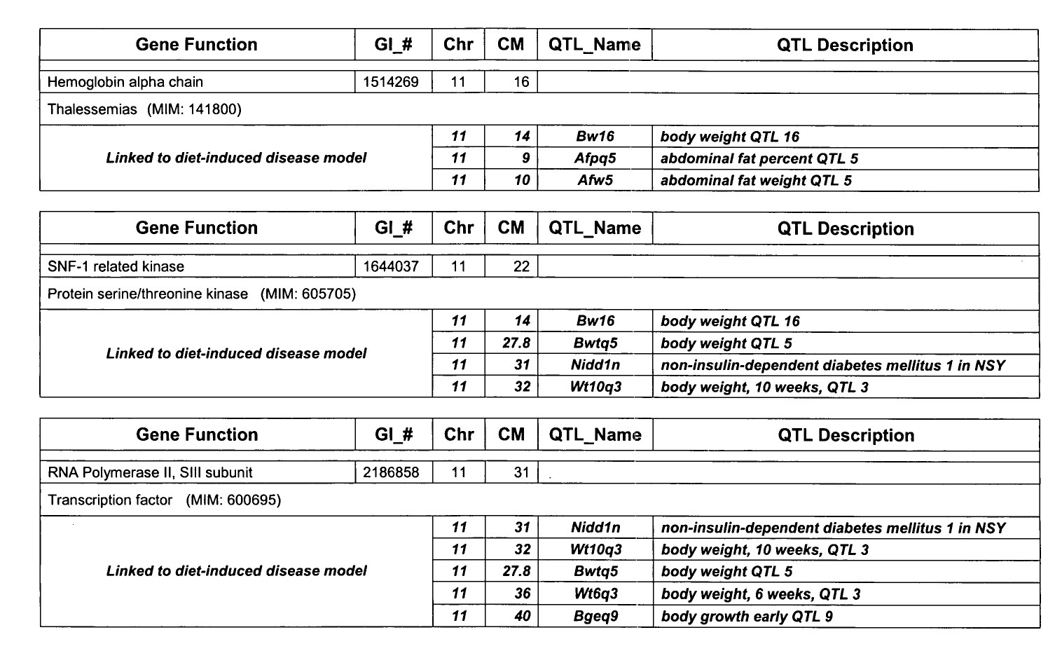

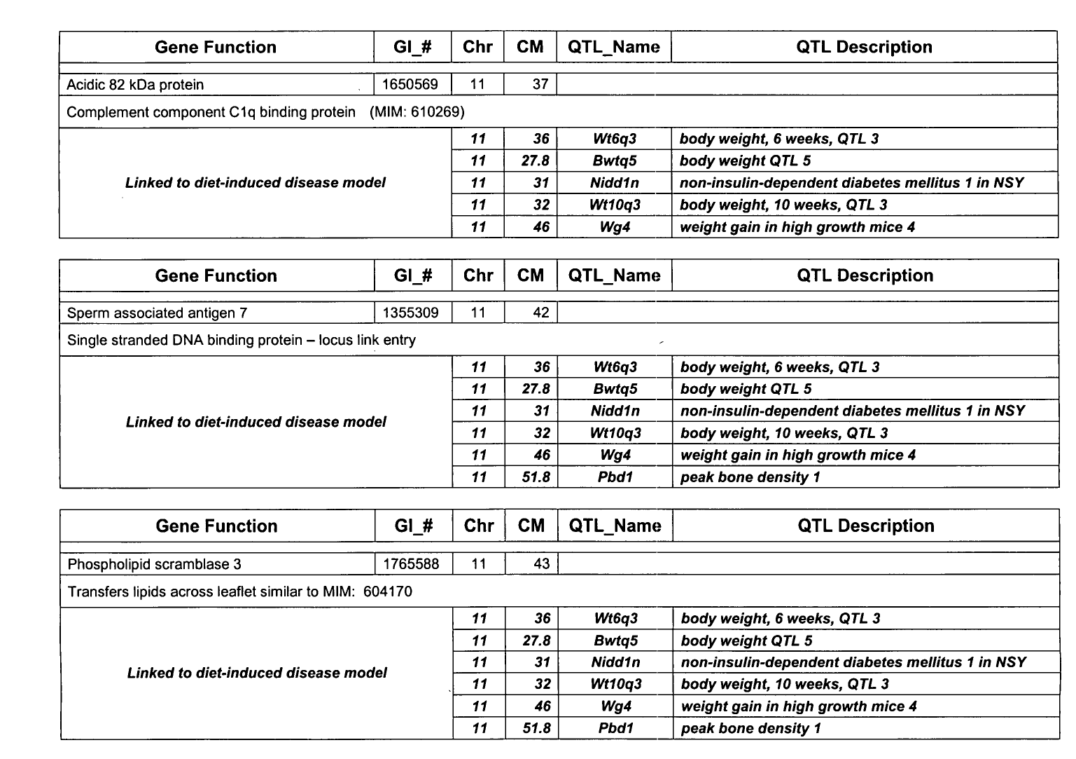

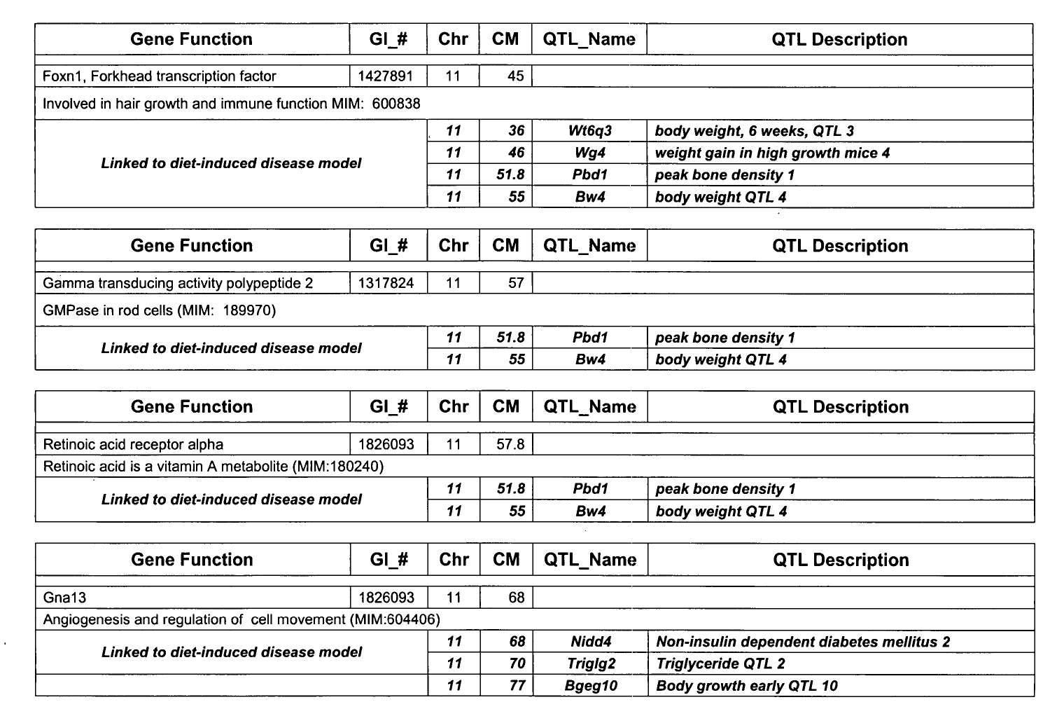

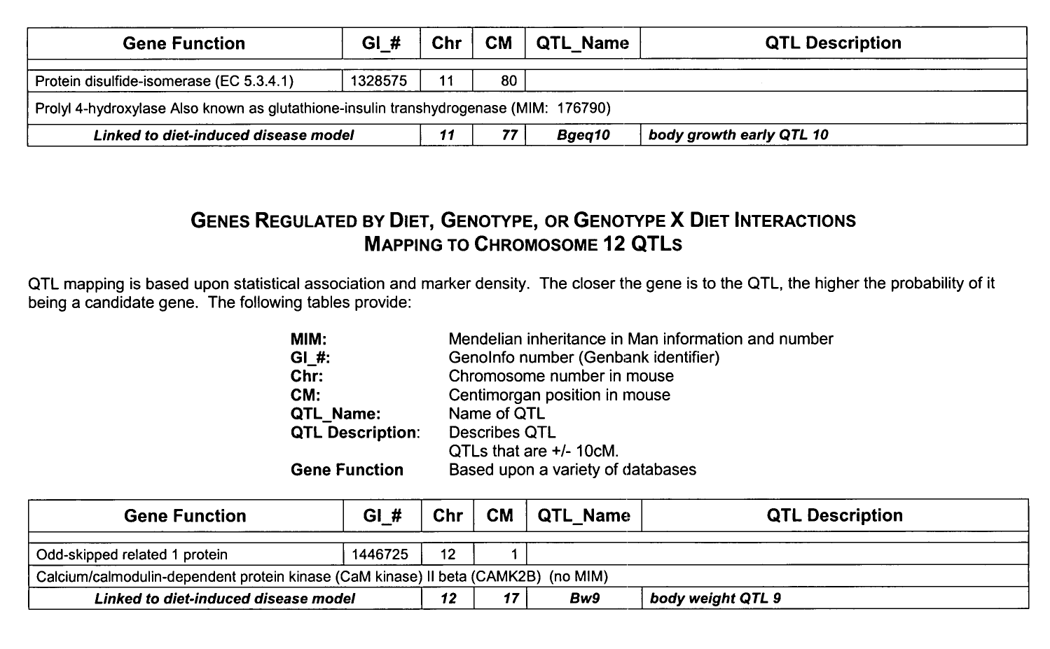

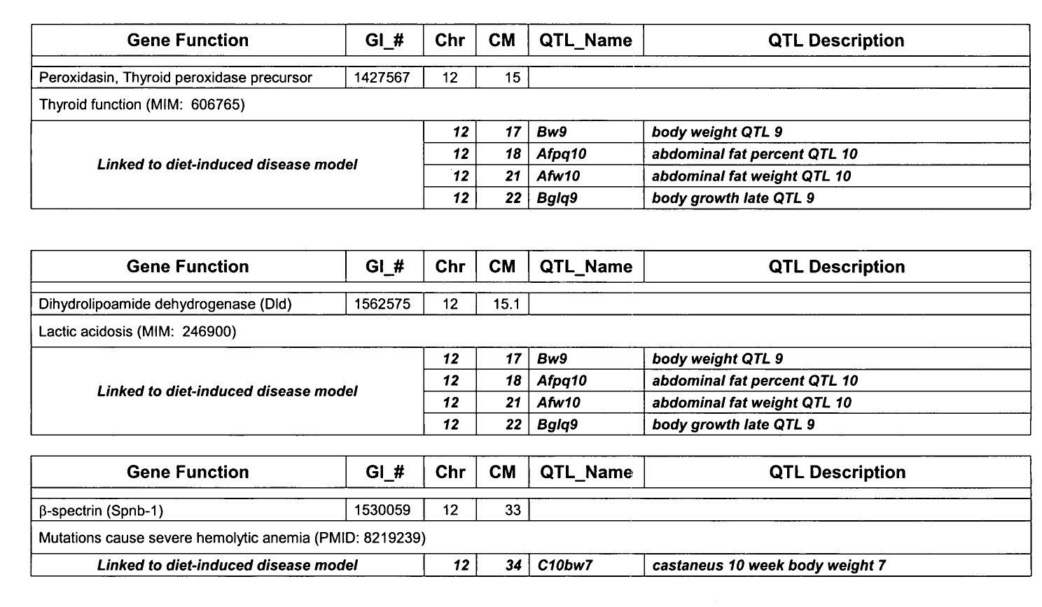

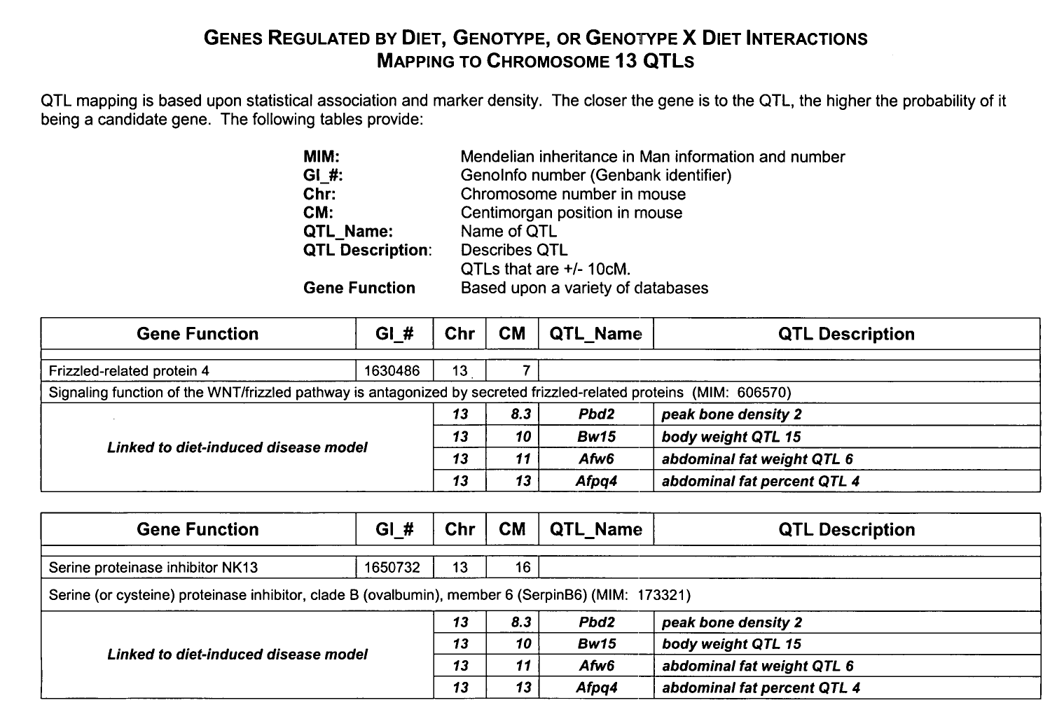

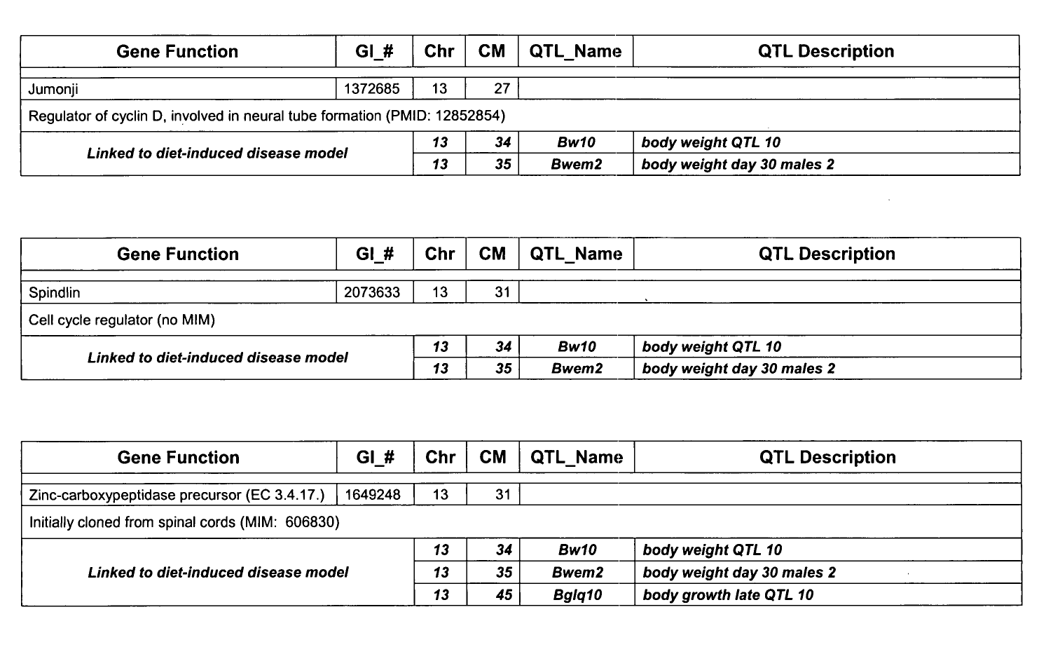

### DETAILED DESCRIPTION OF THE INVENTION

A method for identifying diet-regulated disease-associated polynucleotides by doing the following: Two different inbred genotypes (known genotypes) are selected (A and B). One of these genotypes (A) is more susceptible to a disease (can be any “undesirable” phenotype), and the other genotype (B) is less susceptible to the same disease. Then each genotype is divided into two groups (A1 and A2 and B1 and B2). For one genotype, each group is fed a different diet (A1 is fed diet No.1 and A2 is fed diet No.2, and similarly for B1 and B2). Gene expression is then compared across the strains that differ in either genotype or in diet, but not in both. I.e., A1 is compared with A2; A1 is compared with B1; but A1 is not compared with B2. This is because we want to deal with only one variable at a time. Differential gene expression is identified between the compared groups, and genes are identified that show significant changes in expression (e.g., a 1.5 or 2.0 or 2.5-fold increase or decrease in gene expression. These genes are diet-regulated disease-associated genes. As a further step, these identified genes are then compared against independently-identified diet-regulated and/or disease associated QTL's. This step helps add assurance to the identification and to help differentiate cause from effect for genes that are differentially expressed in response to diet.

More specifically, the method of the invention may be carried out as follows: a) comparing gene expression between two inbred strains in response to different diets, wherein one inbred strain is susceptible to a disease and the other inbred strain is not susceptible to the disease, b) identifying those differentially expressed polynucleotides that overlap with independently-derived diet-regulated QTLs, and c) analyzing the data to identify diet-regulated disease-associated polynucleotides. Generally the disease in question is a diet-associated disease. Gene expression is usually compared by comparing mRNA abundance (for example using a cDNA array), but may be compared by looking at protein levels. Often in-bred strains of mice are used mRNA abundance is compared between strains in response to different diets. Lastly, genes (or EST's or any polynucleotides) that have been identified as being significantly differentially expressed are compared with previously-identified, independently-derived diet-regulated QTLs. Various methods are also disclosed for use in disease screening, monitoring and treatment. The disclosed methods may also be used for formulating medical foods used to treat and prevent disease and slow disease progression. Arrays are also disclosed that employ one or more genes/polynucleotides identified by the method of the invention. Various new diet-regulated disease-associated genes and ESTs are also disclosed, as are compositions of medical foods and dietary supplements that have use for prophylactically treating populations and individuals susceptible to disease, or therapeutically treating populations and individuals who have disease.

Using the method of the invention, gene expression comparisons are made within a strain based upon differences in diet, and between strains fed the same diets. We can therefore identify genes regulated by diet in one or both strains, genes regulated by the one or both genotypes, and genes regulated by the interaction between diet and genotype. That is, certain genes will be regulated without regard to diet—the regulation of these genes will depend upon genetic makeup only. Other genes will be regulated in the same manner in all individuals in the population by what is eaten, although the level of the response may differ among individuals. The regulation of other genes will depend upon an individual's genetic makeup and how it responds to dietary variables. The regulation of these genes therefore differs between individuals even if they eat the same concentrations of dietary chemicals.

Since gene expression differences result from differences in DNA sequence (0.1% difference among humans), the methods of the invention can be practiced by associating genetic differences in the identified genes with disease incidence, severity, or progression. That is, single nucleotide polymorphisms (SNP) or other polymorphisms in the identified gene sequences can be used to identify the variants of diet-regulated genes that are associated with disease or that predict the severity of the disease once diagnosed. The method identifies genes whose abundance (or regulation) is affected by diet. However, a logical and obvious extension is that differences in protein or enzyme activities of these diet-regulated genes are also likely to influence disease development or severity. Analyzing SNPs or other polymorphisms in the promoter and gene may therefore be used in place of expression profiling.

Various embodiments of the invention include the following:

A method for determining the susceptibility of an individual to a disease, wherein said disease involves a diet-regulated disease-associated polynucleotide, the method comprising: screening an individual for the presence and/or expression of a plurality of polynucleotides identified by the method above or by associated polymorphisms in gene sequence, wherein the pattern of expression of polymorphisms of and in said plurality of polynucleotides corresponds with the susceptibility of an individual to a certain disease.

A method for monitoring the progression of a disease in a subject, the method comprising: at a first date, screening an individual for the presence and/or expression of a plurality of polynucleotides identified by the method above; at a second date re-screening the individual for the expression of the same plurality of polynucleotides, wherein a change in polynucleotide expression corresponds with the desirable or undesirable progression of a disease.

A method for treating a subject so as to reduce the risk of the individual developing a diet-associated disease, the method comprising: screening an individual for the presence and/or expression of a plurality of polynucleotides identified by the method above, wherein the pattern of expression of said plurality of polynucleotides corresponds with the susceptibility of an individual to a certain disease; and altering the expression of one or more diet-regulated disease-associated polynucleotides to reduce the risk of the subject developing the disease.

A method for treating a subject so as to reduce the risk of the individual developing a diet-associated disease, the method comprising: screening an individual for the presence and/or expression of a plurality of polynucleotides identified by the method above, wherein the pattern of expression of said plurality of polynucleotides corresponds with the susceptibility of an individual to a certain disease, and altering the diet of the individual so as to reduce the risk of the subject developing the disease.

A method for treating a subject so as to ameliorate a diet-associated disease, the method comprising: screening an individual for the presence and/or expression of a plurality of polynucleotides identified by the method above, wherein the pattern of expression of said plurality of polynucleotides corresponds with the susceptibility of an individual to a certain disease; and altering the expression of one or more diet-regulated disease-associated polynucleotides so as to affect an improvement in the progression of the disease.

A method for treating a subject so as to reduce the progression of a diet-associated disease, the method comprising: screening an individual for the presence and/or expression of a plurality of polynucleotides identified by the method above, wherein the pattern of expression of said plurality of polynucleotides corresponds with the susceptibility of an individual to a certain disease, and altering the diet of the individual so as to affect an improvement in the progression of the disease.

A method for identifying the suitability of various drugs or medical food regimens for a subject diagnosed with a disease, the method comprising: screening an individual for the presence polymorphisms regulated by diet, using the above methods.

A method for identifying genetic susceptibility of a subject to a chronic disease so as to select appropriate drug(s) or diets for reducing the incidence, severity, or progression of the disease or symptoms of the disease, the method comprising: screening an individual for the presence polymorphisms in genes regulated by diet, genotype, or their interactions using the above methods.

In all cases, the method claims the use of individual or combinations of diet-, genotype-, or diet/genotype-regulated genes for diagnostics and/or development of drugs.

The strategy described for identifying genes participating in disease processes uses both gene expression analyses in inbred strains of mice (susceptible to disease and not susceptible to disease) and publicly available QTL information [4, 5, 23-26]. The strategy consists of a multi-step procedure for identifying nutrient-regulated genes involved in disease development in laboratory animals differing in genetic susceptibility to diet-induced disorders. Altering a diet in genetically-susceptible individuals may be a way to slow the initiation and/or change disease progression.

Identification of Diet-Regulated Disease-Associated ESTs and Genes

A multi-step procedure was developed to identify genes regulated by dietary chemicals that participate in disease development.

1. Analyze mRNA abundance in inbred strains of mice in response to different diets using cDNA arrays (variations include measuring protein abundance). One strain of mice used is susceptible to a disease and the other strain is not be susceptible. Diets are chosen to induce chronic diseases such as obesity or diabetes.

2. Compare expression profiles between inbred strains of mice that differ in susceptibility to diet-induced disease. Differences in mRNA abundance identify genes regulated by genotype, diet, and their interactions. Since the diet is chosen to induce disease development in susceptible genotypes, a subset of these genes will be involved in disease development.

3. Differentiate between cause from effect genes by determining the map position of diet-regulated to independently-derived QTLs. Differentially expressed genes that overlap QTLs become candidate genes for the disease (an example of association mapping [16]).

4. Characterize the expression and activity of a subset of the genes and proteins that were identified by expression array technology and QTL analyses in animal models of the disease

5. Identify polymorphisms in candidate disease genes

6. Examine their associations in humans who are healthy vs. those showing symptoms of the disease.

Detailed Methodology

Animals, diets and protocols. Male or virgin female (eliminates complications and effects of pregnancy) mice of defined genotype are fed a semi-purified diet containing 4% corn oil for 1 wk and then randomly assigned to control or experimental diets for at least 2 wks and up to the normal lifespan of the mouse. Each diet contains 1.4% of the respective total oil content as soybean oil to assure adequate fatty acid content (NRC 1995, [27]). An example of the diets is shown in Table 2. The diets are formulated according to modified AIN-76 guidelines (NRC 1995,[27]), were pelleted, and color coded by a commercial vendor. The diets are balanced for minerals, vitamins, fiber, and protein, and differ in carbohydrate and lipid level. In some cases, individual chemicals (natural or man-made) can be added to the diet to determine their effect on gene expression.

Mice are caged and fed individually with free access to food and distilled water in temperature-controlled rooms maintained at 23±1° C. with a 12-h light:dark cycle. Animal care meets National Institutes of Health guidelines. Food spillage is also monitored throughout the course of the experiment. Efficiencies of energy utilization are calculated from the recorded weekly weight gain/calculated weekly energy intake.

At the end of the feeding period, all mice were deprived of food for 12 h and offered a preweighed 3-g pellet of their assigned diet. After 2 h, the uneaten food was removed; at a defined time after, all mice were injected intramuscularly with 0.02 mL/g body weight of ketaset/xylazine mixture (Ketaset, Fort Dodge Laboratories, Ft. Dodge, Iowa) for collection of blood via cardiac puncture. Immediately thereafter, they are killed by cervical dislocation and their livers and hearts removed, individually frozen in liquid nitrogen, and stored at 80° C. for mRNA isolation. Altering the length of time for depriving food, for length of feeding, and for time to collection would all be trivial and obvious modifications of this protocol.

TABLE 2
Diet composition (exemplary)

| | Corn | | Coconut | |
|---|---|---|---|---|
| | 4% | 20% | 4% | 20% |
| Ingredient | g | | | |
| Corn oil | 2.8 | 18.6 | 0 | 0 |
| Coconut oil | 0 | 0 | 2.8 | 18.6 |
| Soybean oil | 1.4 | 1.4 | 1.4 | 1.4 |
| Maltodextrin | 11.8 | 14.1 | 11.8 | 14.1 |
| Cornstarch | 45.0 | 19.3 | 45.0 | 19.3 |
| Sucrose | 9.5 | 11.2 | 9.5 | 11.2 |
| Casein, alcohol extracted | 19.0 | 22.6 | 19.0 | 22.6 |
| L-Cystine | 0.3 | 0.3 | 0.3 | 0.3 |
| Cellulose | 4.7 | 5.6 | 4.7 | 5.6 |
| Mineral mix¹ | 1.0 | 1.1 | 1.0 | 1.1 |
| Calcium phosphate | 1.2 | 1.5 | 1.2 | 1.5 |
| Calcium carbonate | 0.6 | 0.7 | 0.6 | 0.7 |
| Potassium citrate, monohydrate | 1.6 | 1.9 | 1.6 | 1.9 |
| Vitamin mix¹ | 1.0 | 1.1 | 1.0 | 1.1 |
| Choline bitartrate | 0.2 | 0.2 | 0.2 | 0.2 |
| Total grams | 100.1 | 94.2 | 100.1 | 94.2 |
| kJ/g² | 15.9 | 19.2 | 15.9 | 19.2 |
| Protein, kJ % | 20.0 | 20.0 | 20.0 | 20.0 |
| Lipid, kJ % | 10.0 | 40.0 | 10.0 | 40.0 |
| Carbohydrate, kJ % | 70.0 | 40.0 | 70.0 | 40.0 |
| Minerals, mg/kJ | 2.7 | 2.6 | 2.7 | 2.6 |
| Vitamins, mg/kJ | 0.6 | 0.6 | 0.6 | 0.6 |
| Cellulose, mg/kJ | 3.0 | 2.9 | 3.0 | 2.9 |

¹AIN-76A diet recommendations (National Research Council 1995).
²Amount of energy is 16.7 kJ/g for protein and carbohydrate, 37.6 kJ/g fat.

Molecular technologies. All molecular technologies, such as RNA isolation, RNA analyses on DNA or microarrays, or proteomic analyses, are standard.

One of the important unique features of the present invention is that gene expression comparisons are made within a strain based upon differences in diet, and between strains fed the same diets. We can therefore identify genes regulated by diet in one or both strains, genes regulated by the one or both genotypes, and genes regulated by the interaction between diet and genotype. Our proof of principle data demonstrated that we identify genes regulated by each of these factors (strain-specific, diet-specific, genotype/diet interaction-specific, and genotype-specific). To our knowledge, no other experimental paradigm is capable of this type of analyses. Our strategy can also determine whether known candidate disease genes are regulated by dietary factors.

### EXAMPLE

Our most complete gene expression study was designed to identify mammary tissue genes regulated by caloric restriction and their role in mammary cancer development. There is considerable evidence accumulating that caloric restriction reduces the incidence and severity of several diseases, including breast cancer. The strains used were obese yellow A^{vy}/A and agouti A/a that are segregants (i.e., littermates) of a (BALB/cSTCrlfC3H/Nctr×VYWffC3Hf/Nctr-A^{vy} mating. The A^{vy} mutation is caused by insertion of an intracisternal A particle genome in the promoter of the agouti (A) gene. It might be considered one of the first (albeit naturally occurring) transgenic mouse strains. The 131 amino acid long agouti gene product is ectopically expressed resulting in yellow coat color, obesity, hyperinsulinemia, and increased risk of spontaneous and chemically induced mammary cancer. Since A^{vy}/A become obese (˜60 g v ˜25 g for A/a mice) and develop symptoms of Type II diabetes, we used cDNA arrays to analyze genes regulated in liver in response to genotype, diet (100% vs 70% calories), and the interaction between diet and genotype.

Experimental details: Fifty-two mice of each strain fed ad libitum and 52 mice of each genotype fed restricted diets (70% calories) in the control group. Mice were fed a defined diet (not shown but available upon request) for 30d, 60d, 90d, and 1 yr—with 5 mice per group. Another set of 394 animals were used for a mammary carcinogenesis experiment. Our specific feeding regimen was used during the last day of the experimental period to ensure that all animals were in a fed state at death. Liver, mammary, pancreas, kidney, heart, colon, and intestine were removed from each mouse and are available for gene expression analyses. Phenotypic characterizations were conducted (e.g., food intake, body weights) since they may be correlated with expression of one or more genes. mRNA from livers were isolated, converted to radioactive cDNA, and used to probe for Genome Systems membranes spotted with ˜18,000 mouse genes.

Liver Gene Regulation Results: Gene regulation was compared in duplicate between (read across columns):

| Genotype/Calories | vs | Genotype/Calories | Regulation by: |
|---|---|---|---|
| Obese (A^{vy}/A) - 100% | vs | Obese (A^{vy}/A) - 70% | Diet |
| Normal (A/a) - 100% | vs | Normal (A/a) - 70% | Diet |
| Obese (A^{vy}/A) - 100% | vs | Normal (A/a) - 100% | Genotype |
| Obese (A^{vy}/A) - 70% | vs | Normal (A/a) - 70% | Genotype |

For each pair, expression levels of 18,000 different genes were compared. The comparisons were done in duplicate. Approximately 600 genes changed expression levels (mRNA) greater than or equal to 2.5 fold (up-regulated) or greater than or equal to 1/2.5 fold* (down-regulated) between diets or genotypes in the replicate experiments. Thus the abundance of particular mRNAs was 2.5 times more in one condition and/or genotype combination relative to another condition and/or genotype. The inventors chose a factor of 2.5 as a threshold in the change in gene expression (mRNA) to determine a significant change in expression, but the method may be performed using any number of different thresholds, depending on the sensitivity and specificity required. The threshold increase in gene expression to be used to determine a significant change, and therefore a potentially important target gene/EST may be determined by routine experimentation by one of skill in the art, and may, for example, be 1×, 1.5×, 2×, 2.5×, 3×, 3.5×, 4×, 4.5×, 5×, 6×, 7×, 10×, 15×, 20×, 30×, 50× or more. *(4 groups were used: A1, A2, B1, B2. The following comparisons were done to create ratios: (1) A1/A2 and A2/A1; B1/B2 and B2/B1; A1/B1 and B1/A1; A2/B2 and B2/A2. All of these ratios had to be equal to 2.5 or greater than 2.5 to be ncluded for the gene to be considered statistically significant. A short form for writing all of these combinations is to use the reciprocal for the second comparison; which in the present example is the reciprocal of 2.5 which is 1/2.5 (instead of saying A1/A2 and A2/A1 just say A1/A2 and its reciprocal). Mathematically the relationship is statement is: 1/2.5=<A1/A2>=2.5.)

Of the 601 genes, 376 have been previously identified. The genes were sorted into functional groups (Table 2).

TABLE 2
Functions of Diet- and Genotype-Regulated Genes

| Function | Number |
|---|---|
| Structural Proteins | 71 |
| Metabolic Enzymes | 82 |
| Proteases/Inhibitors | 19 |
| RNA interacting proteins | 12 |
| Signal Transduction/Transcription Factors | 98 |
| Unknown Function | 41 |

The map position of each of the 601 diet-regulated genes was determined using publicly-available chromosomal maps at the Jackson Laboratory (www.jax.org) and compared to independently-derived quantitative trait loci. 117 of the diet- and genotype-regulated genes mapped to diabetes, obesity, and/or growth QTLs (some of these loci overlapped). Of these 117 genes, 41 were involved in signal transduction and/or transcriptional regulation processes (Table 3). All or a subset of these genes will be used as diagnostics for type 2 diabetes and obesity, and a subset are candidate drug targets. 32 genes were identified as being associated with diabetes and are shown in the dada set.

Genes Regulated by Genotype

The sum total of gene expression of all genotype-regulated genes contributes to physiological differences between A^{vy}/A and A/a mice. Fifteen (15) genes were regulated at 2.5 fold (or 1/2.5 fold) difference by genotype regardless of caloric intake. Several notable examples illustrate how genotype alters expression of key genes and therefore phenotype:

Snrk, SNF-1 related kinase was more abundant in A/a relative to A^{vy}/A. SNF1 is involved in chromatin remodeling by modifying histone H3.

DNA methyltransferase (Dnmt1) mRNA was more abundant in A^{vy}/A relative to A/a mice. In general, methylation marks transcriptionally inactive DNA and imprinting. Regulation of abundance of Dnmt1 mRNA by diet may be one of the underlying mechanisms that increases the susceptibility to hepatocellular hyperplasia and neoplasms in A^{vy}/a mice.

SLC22A 7, or the organic anion transporter (Oat), transports glutarate and prostaglandin E2. It is an imprinted gene. PGE2 has been shown to suppress expression of fatty acid synthase, 1-pyruvate kinase, and the S14 protein mRNA in liver.

Diet-Regulated Genes

Genes differentially regulated by calorie intake may play a role in the initiation or increased severity of the disease. Akt2 (PKB), a protein kinase is regulated by diet in the nonsusceptible genotype and the lack of similar regulation in the susceptible genotype may contribute to disease development. Akt2 has been proposed as a signal from the insulin receptor to insulin response elements of target genes. Mice lacking Akt2 are hyperglycemic, hyperinsulinemic, and their livers produce glucose in the presence of insulin.

Gene products with differential abundance in each genotype may also alter energy metabolism.

Tpo, peroxidasin (aka, thyroid peroxidase, deiodinase), was diet regulated in the nonsusceptible genotype. The thyroid prohormone T4 is converted to active T3 or inactive reverse T3 in peripheral tissues by members of the peroxidasin (deiodinase) family of genes. A variant in human deiodinase gene is associated with insulin resistance.

Mknk1 is stress-activated map kinase interacting protein 1. The stress-activated kinase pathway is implicated in regulating genes involved in energy metabolism.

Hipk2, homeo domain interacting kinase, is a component of a corepressor complex regulating the homeobox gene product NK3. The other members of this complex are Groucho (a corepressor protein) and histone deacetylase, suggesting that Hipk2 plays a role in chromatin remodeling and transcriptional regulation.

Rbp1, retinol binding protein, is the serum carrier of retinol. Retinol metabolites are transcriptional regulators that play key roles in energy metabolism and are linked to development of diabetes and obesity.

Genotype+Diet Interactions Genes

The majority of gene products (170 out of 376) were regulated by genotype plus diet interactions: that is, they were regulated differently depending upon combinations of genetic makeup (genotype) and dietary intake.

Genes Mapping to Diabetes QTL

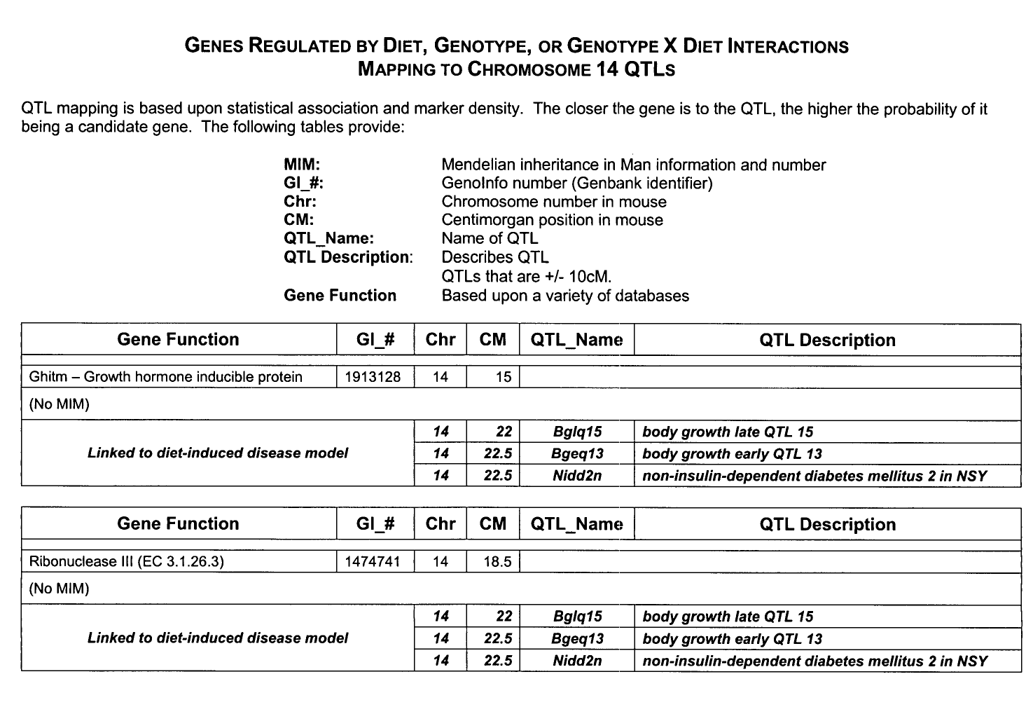

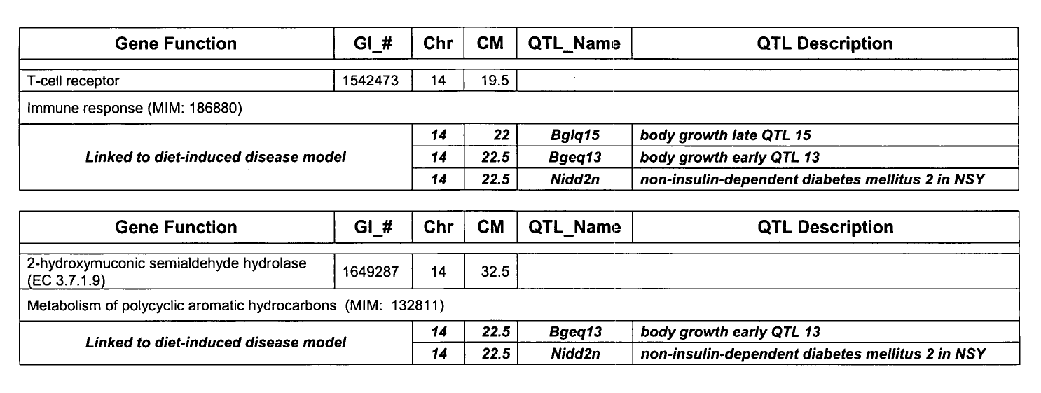

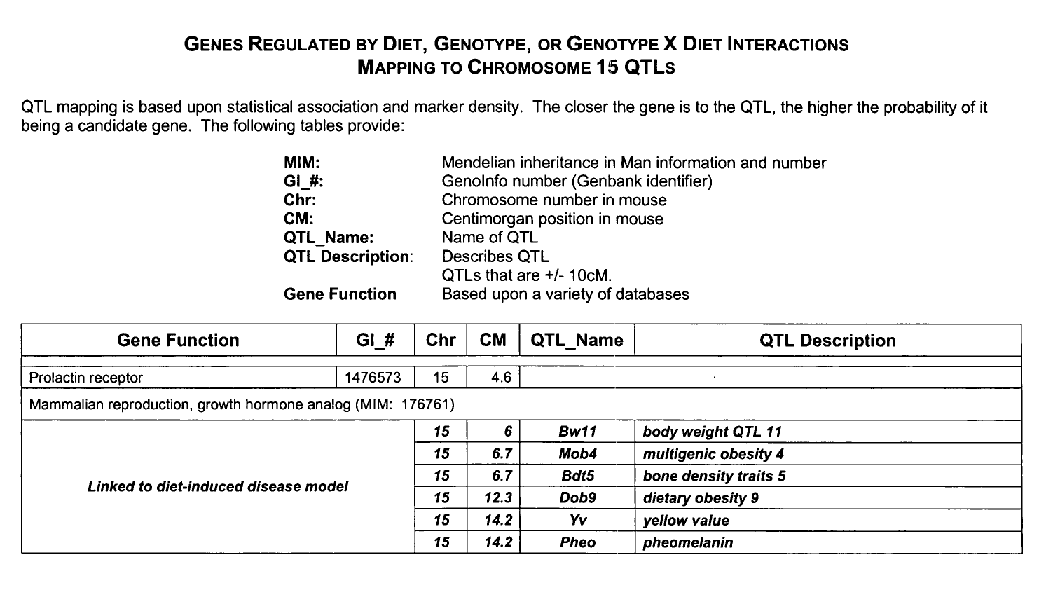

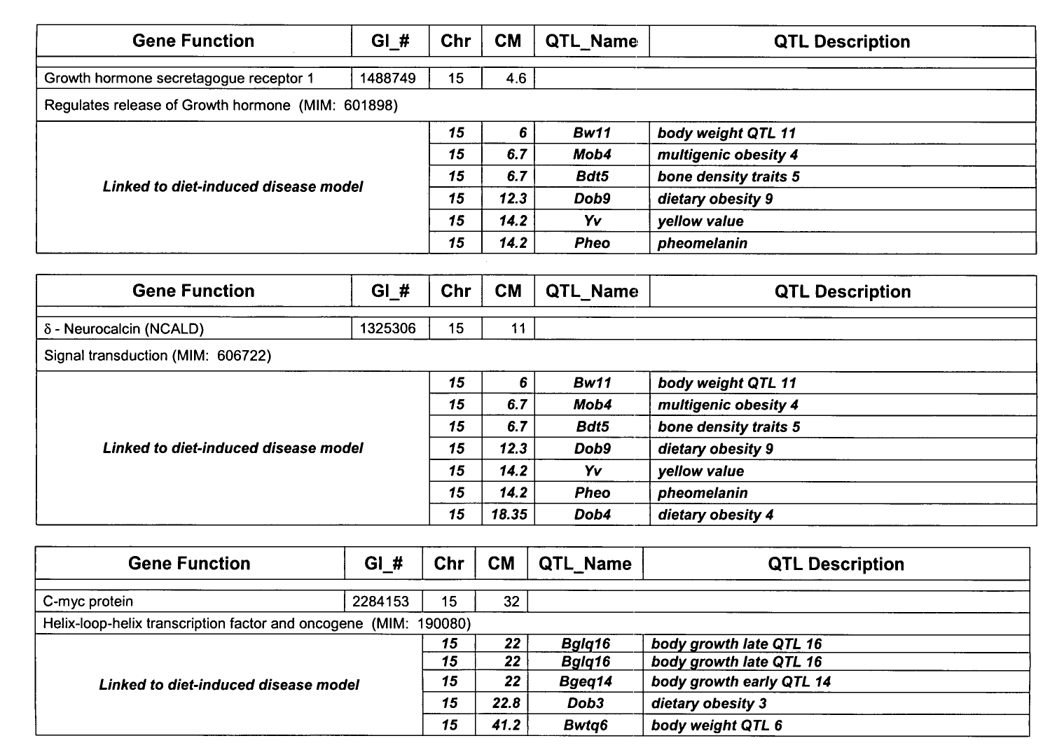

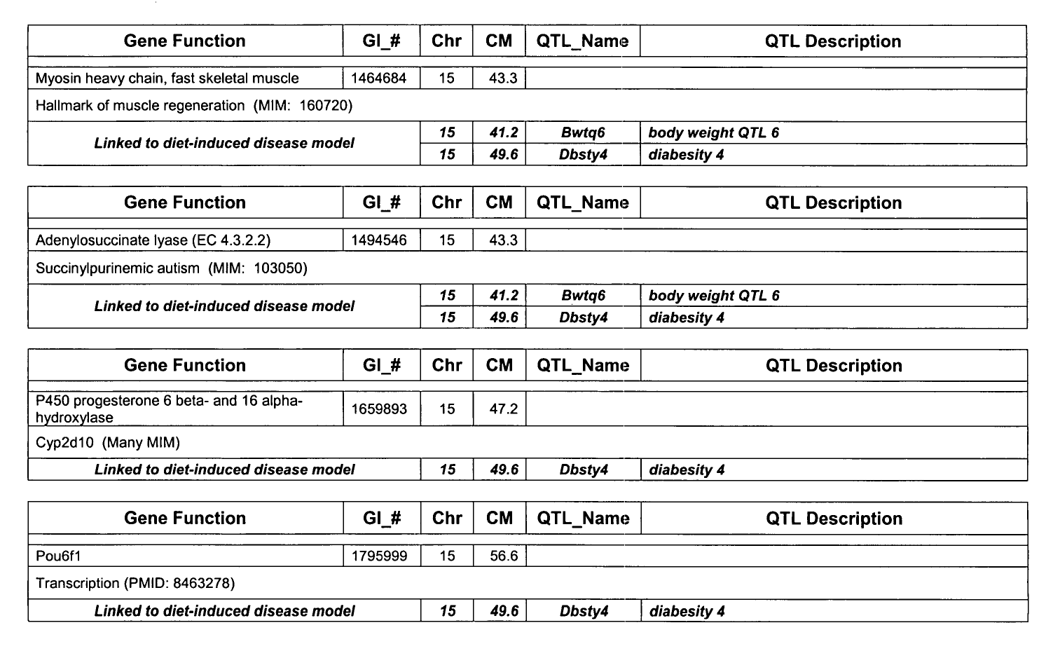

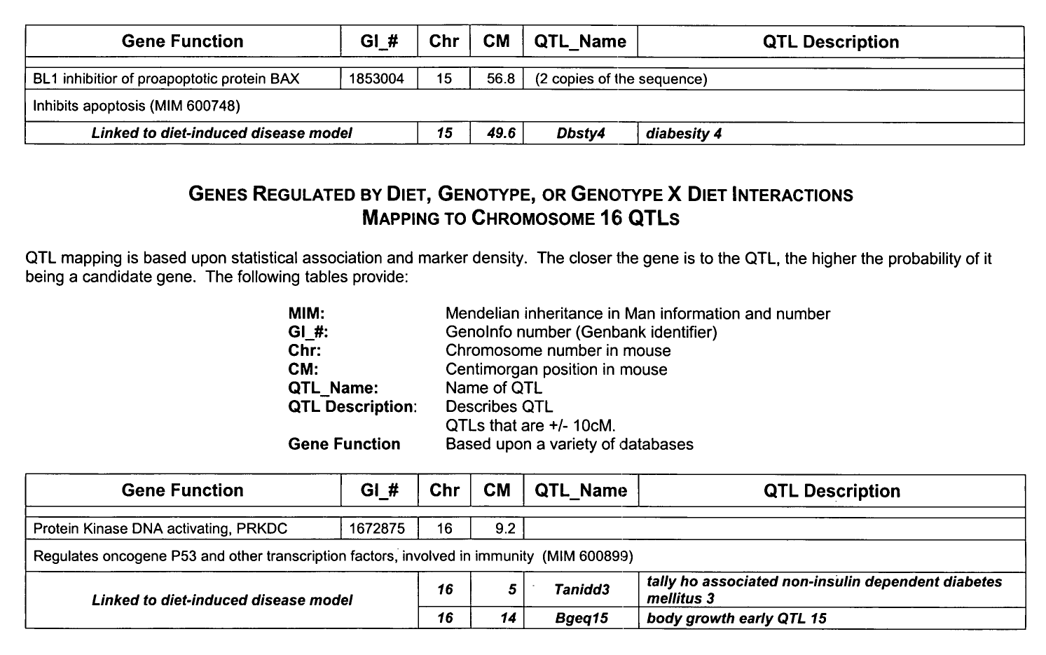

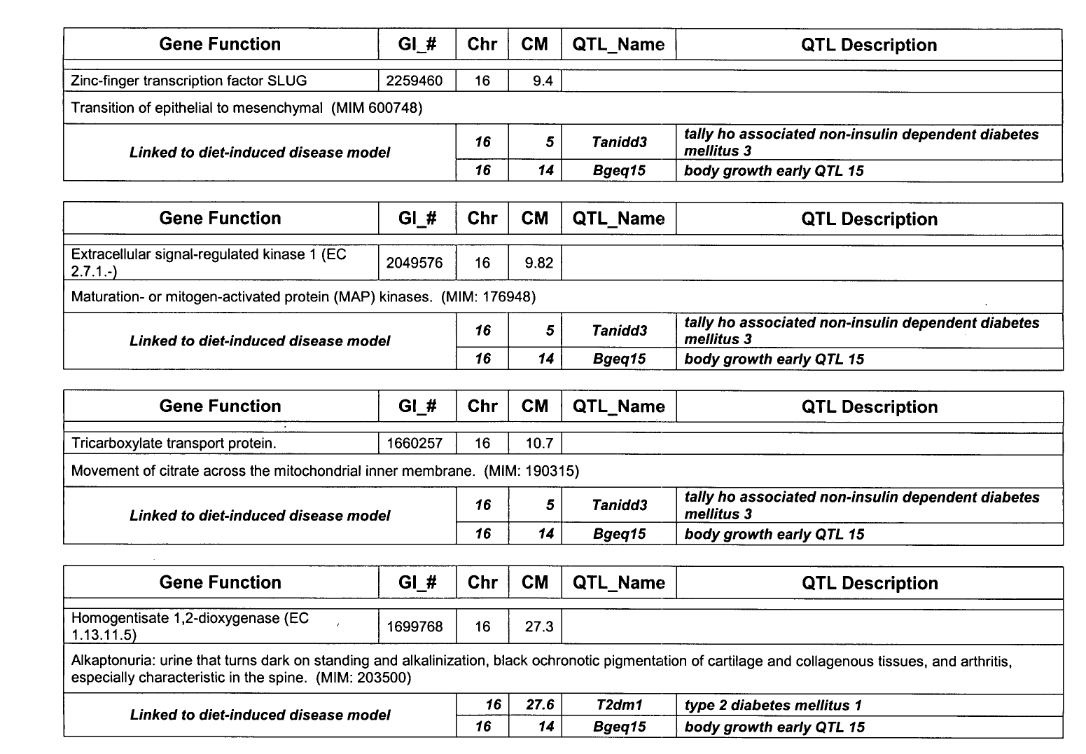

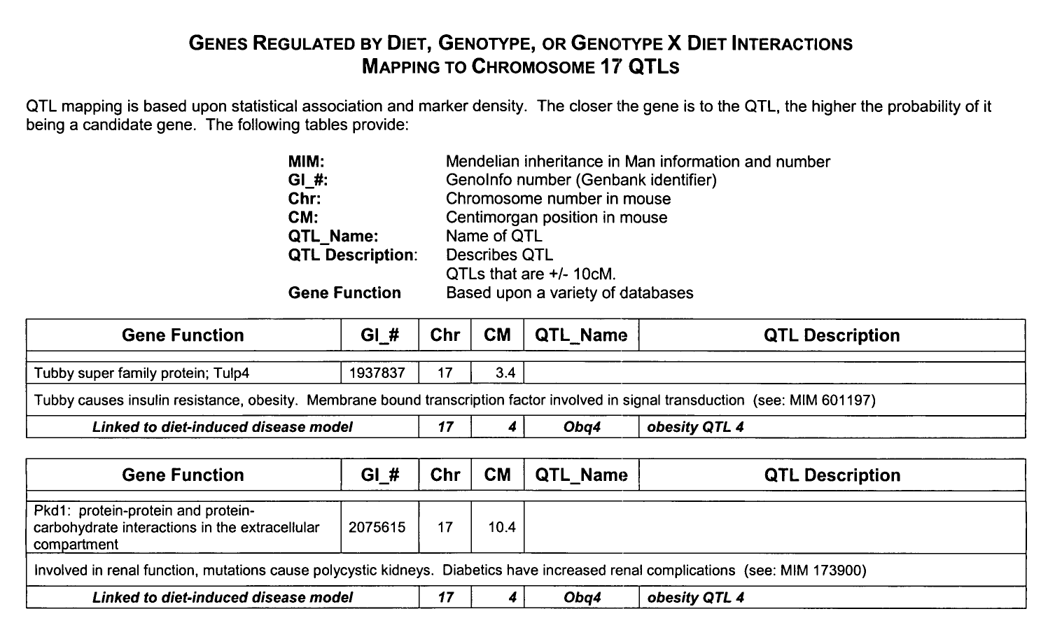

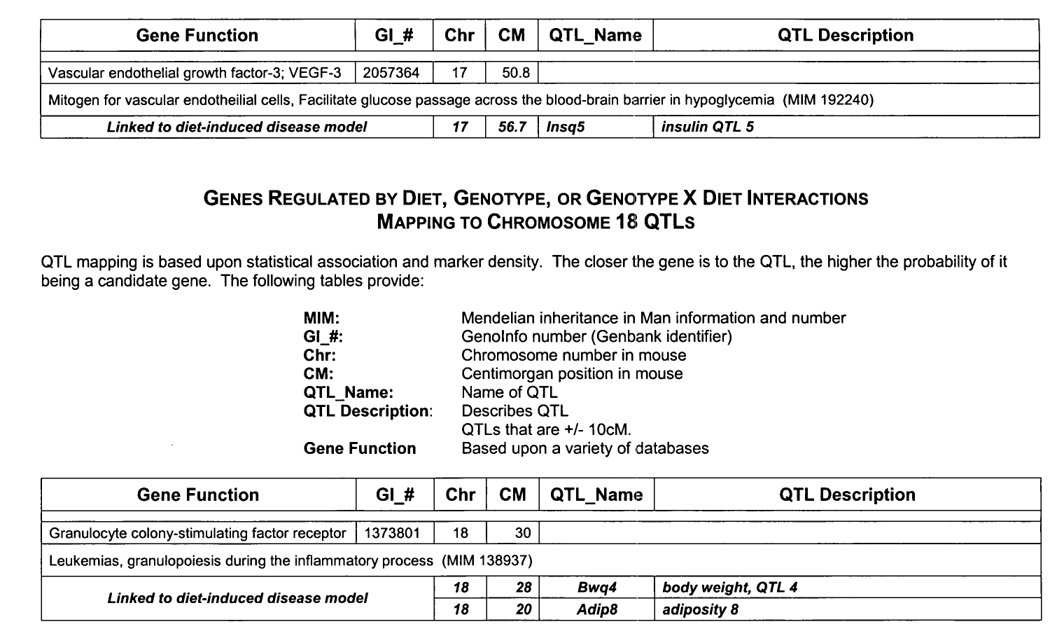

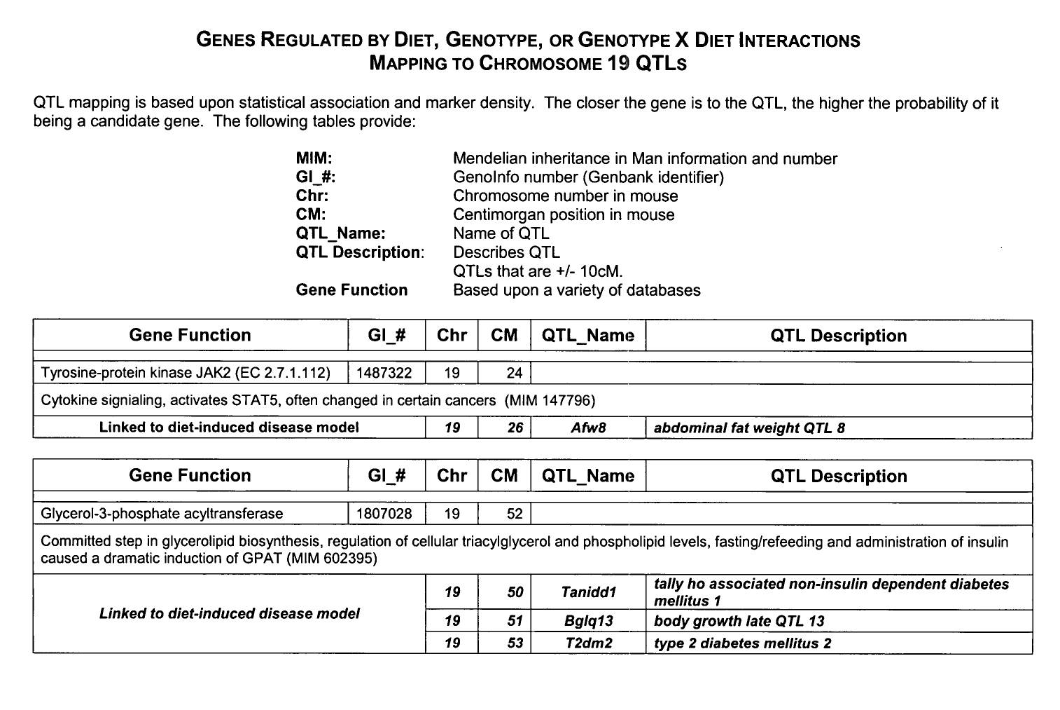

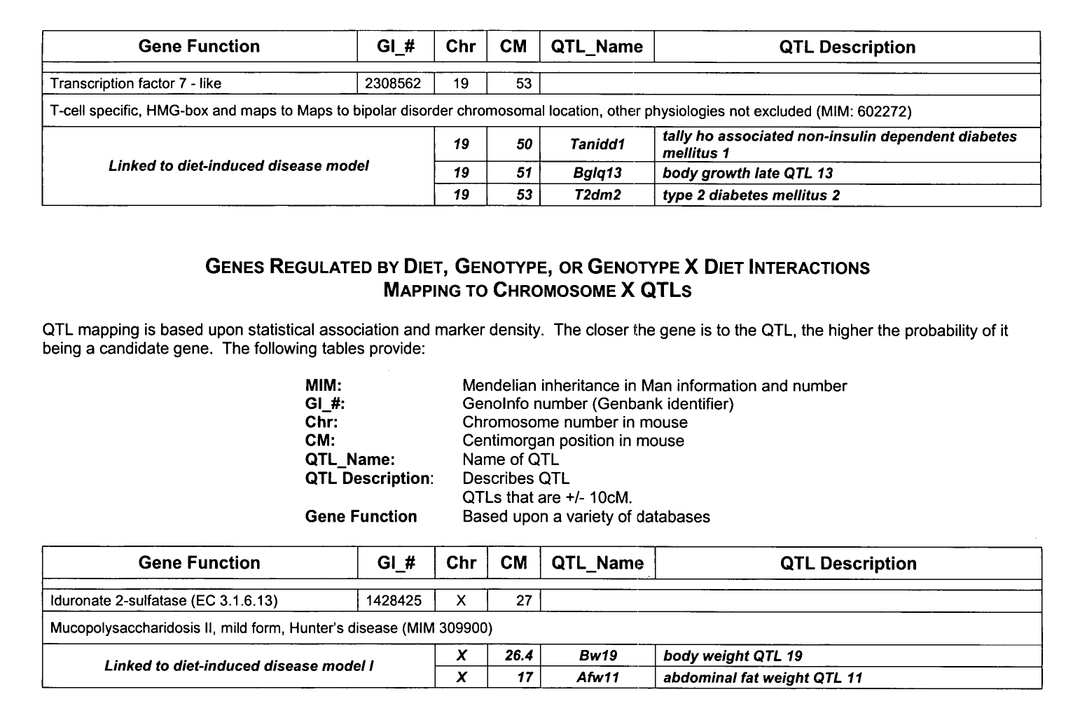

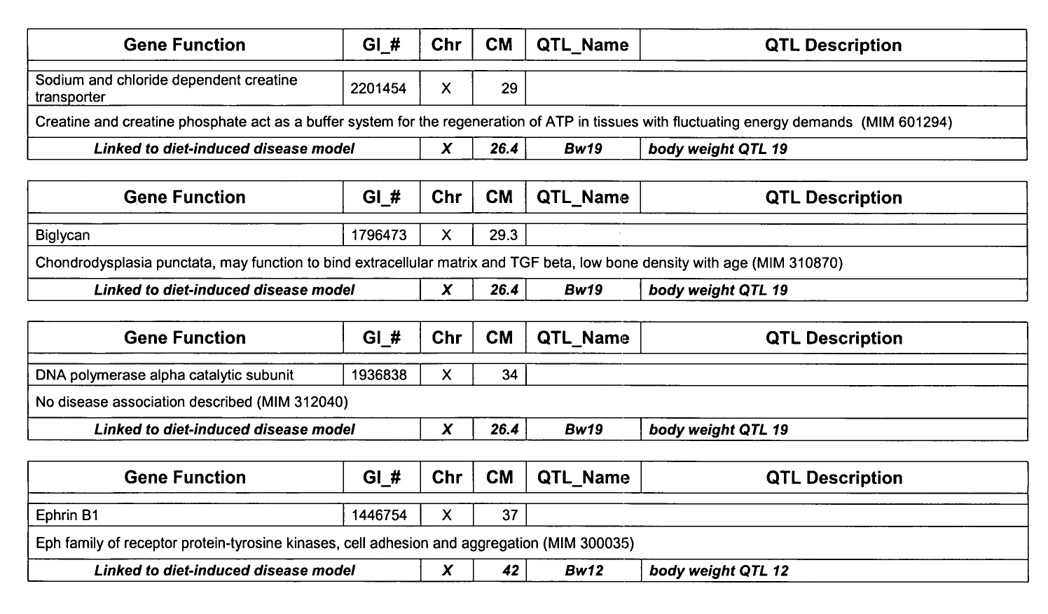

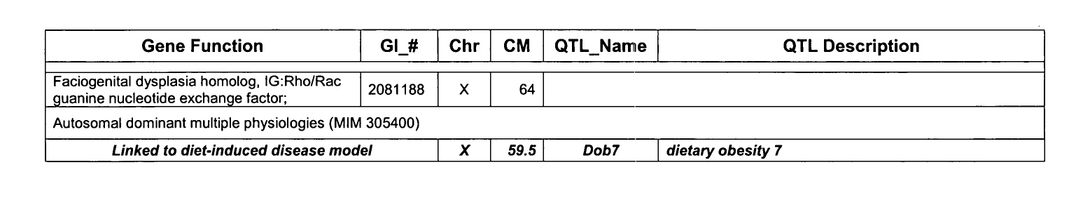

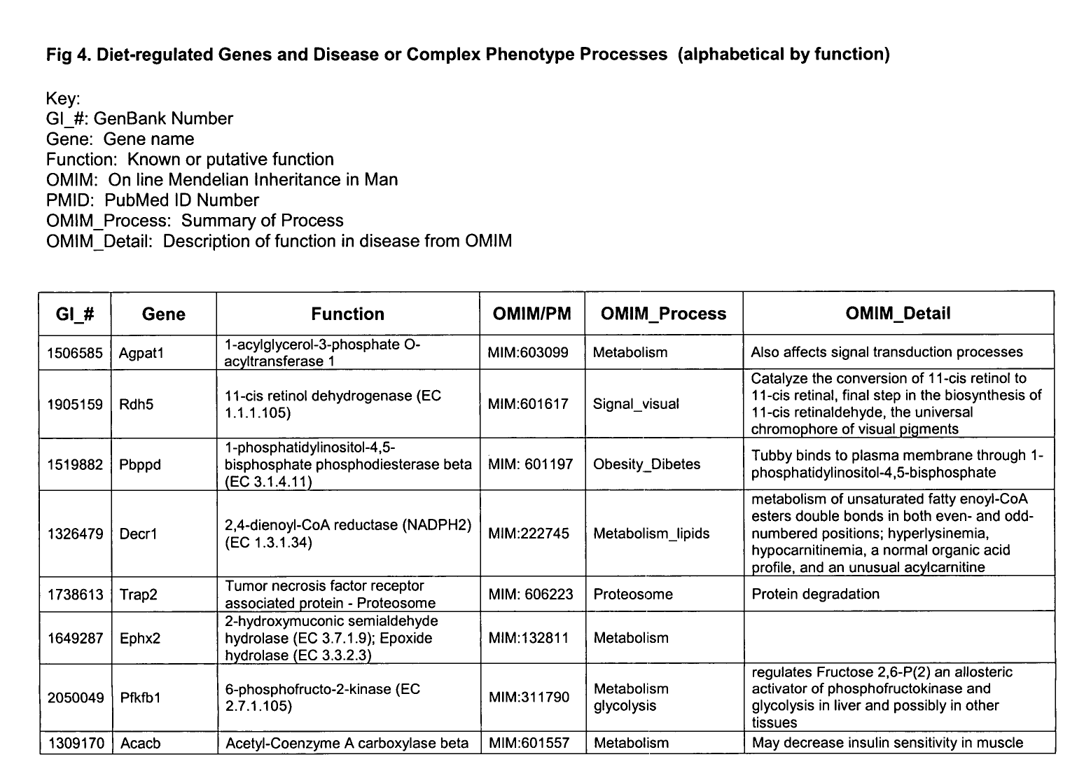

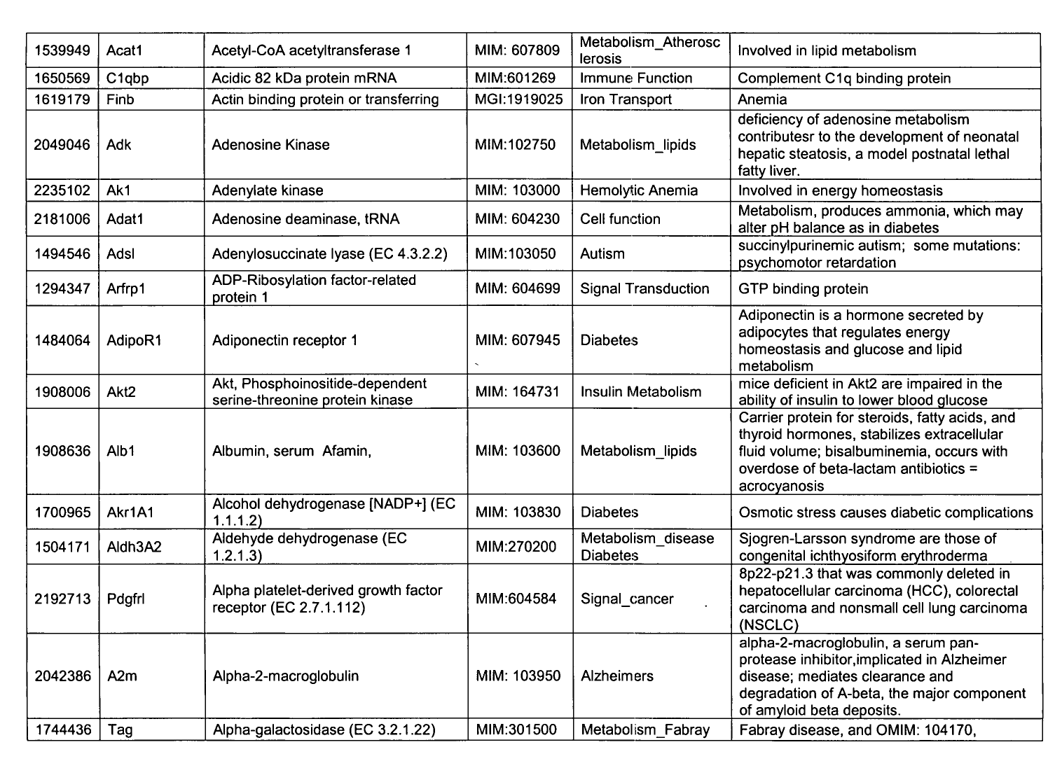

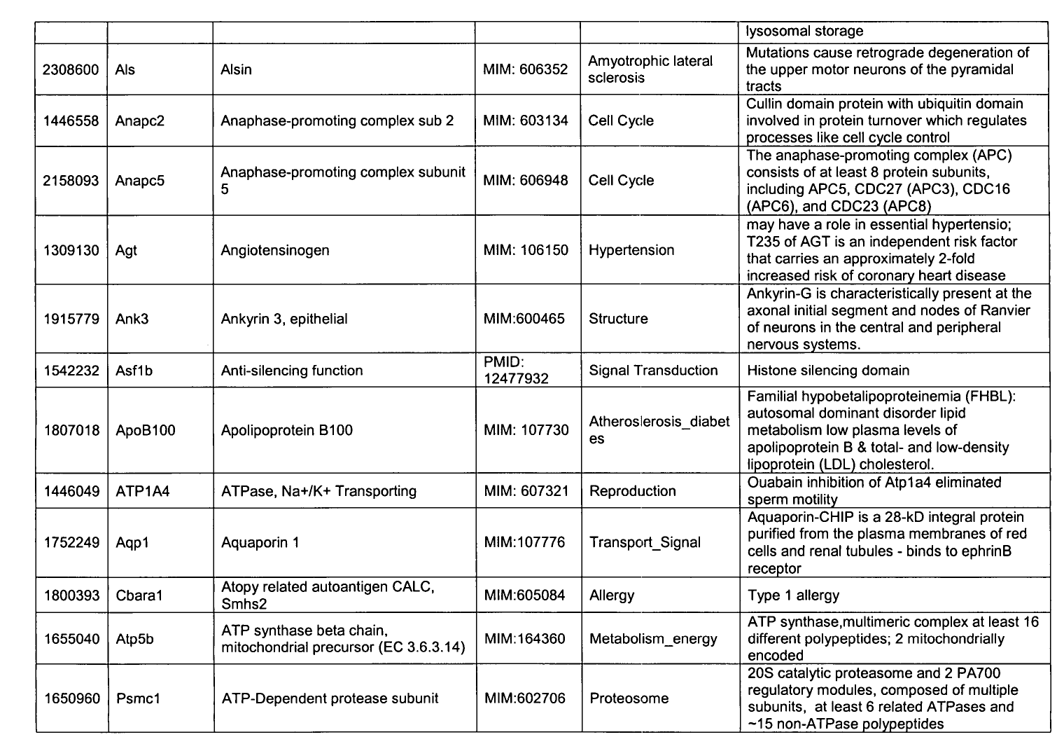

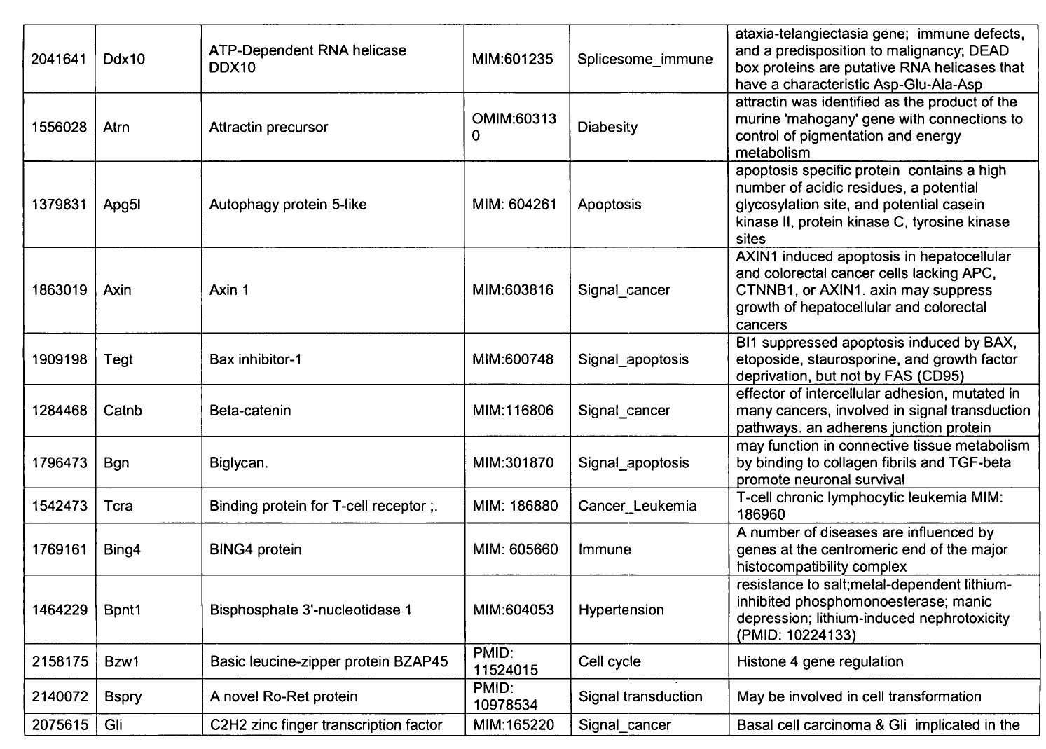

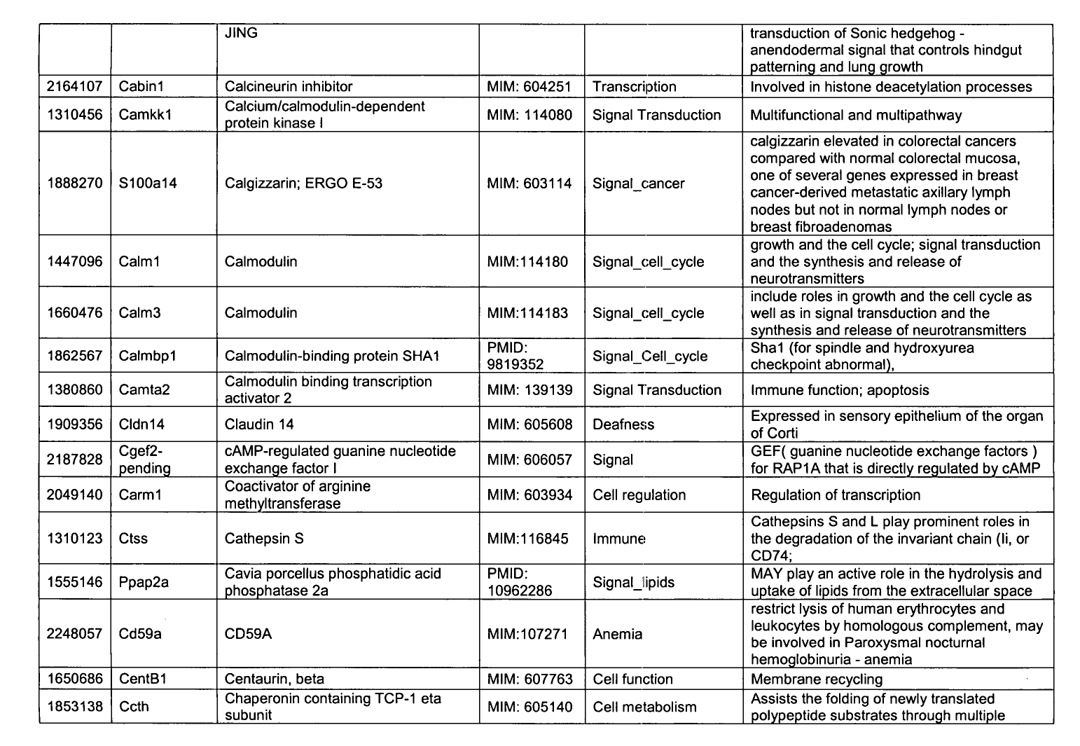

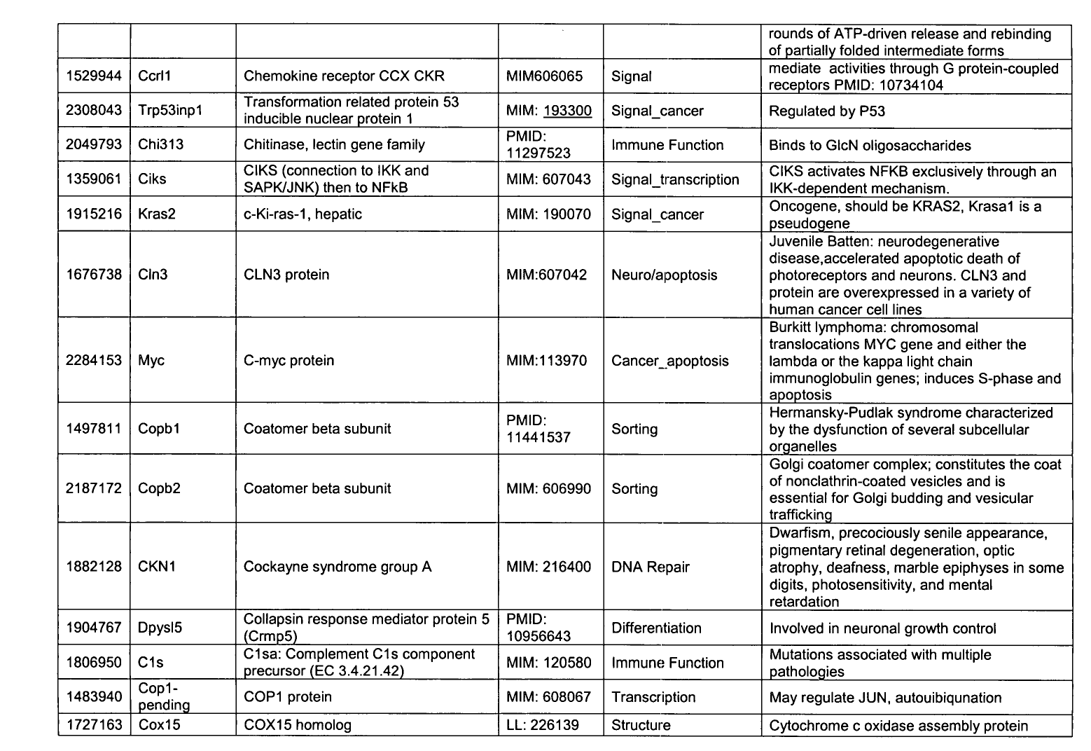

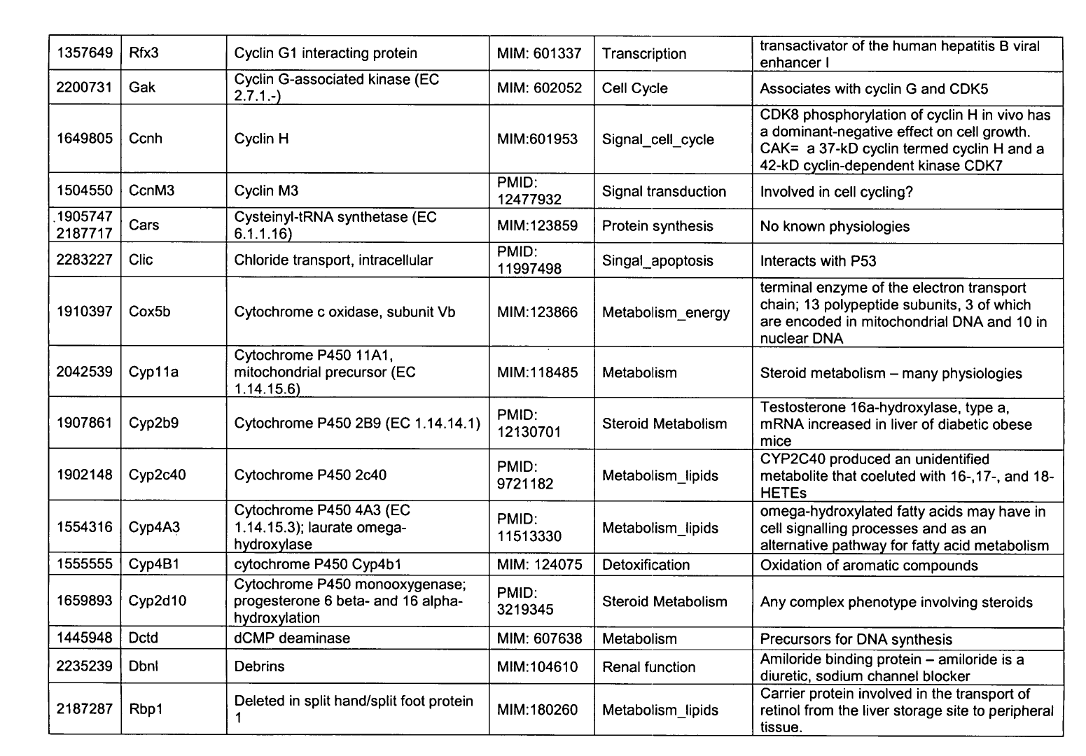

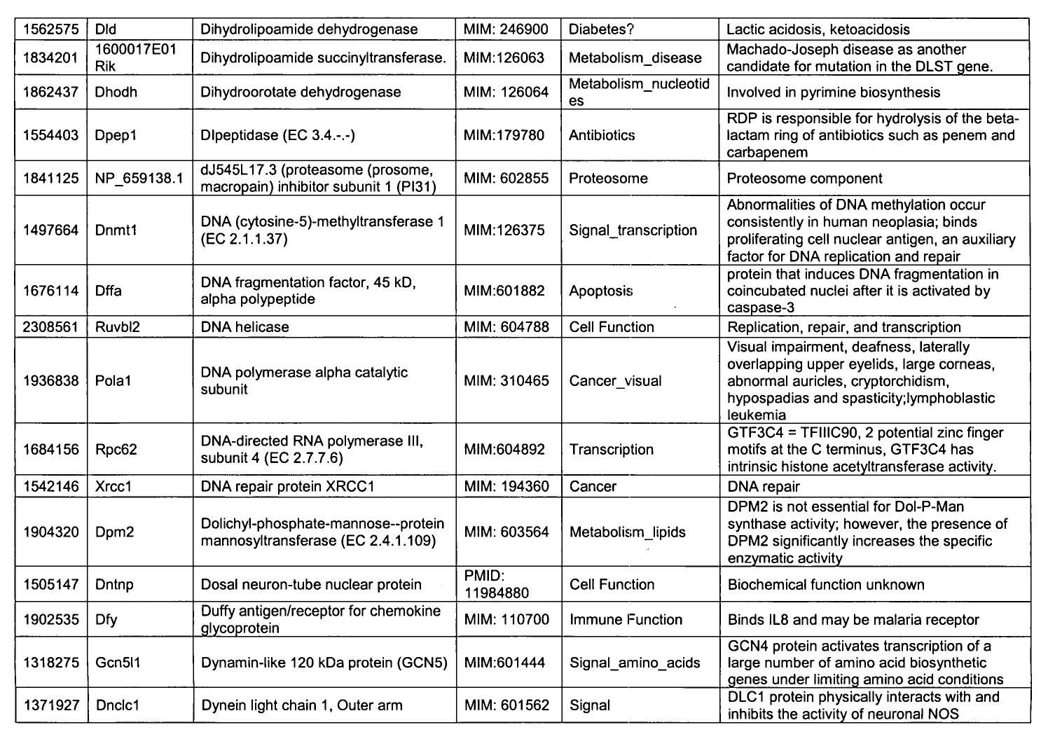

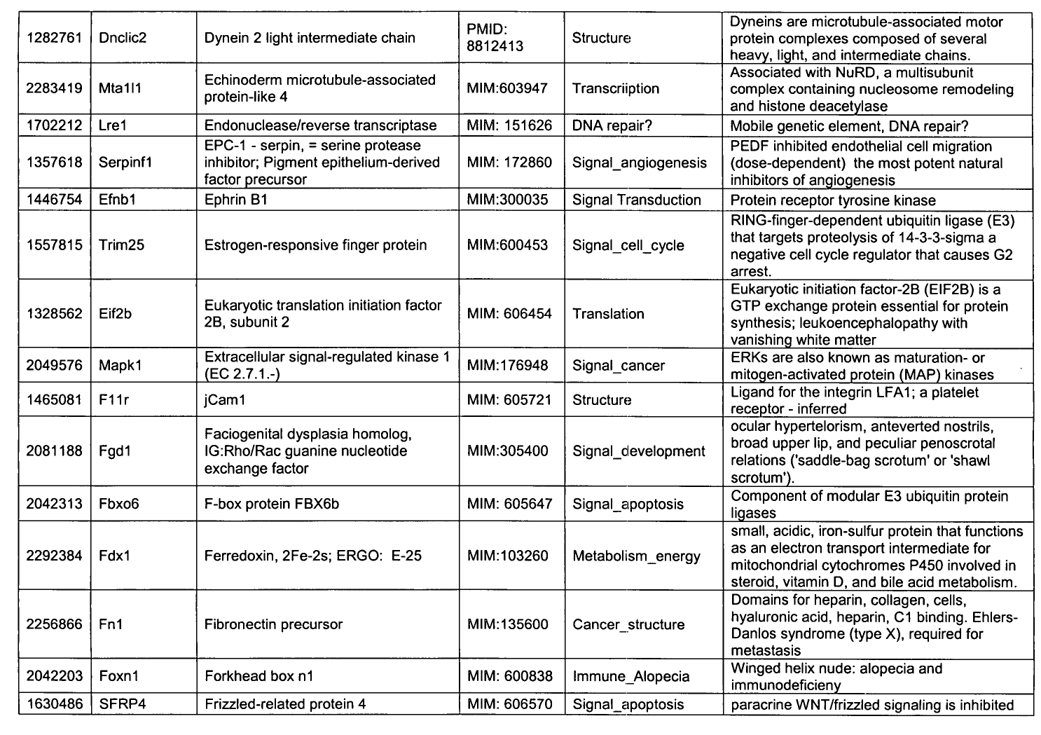

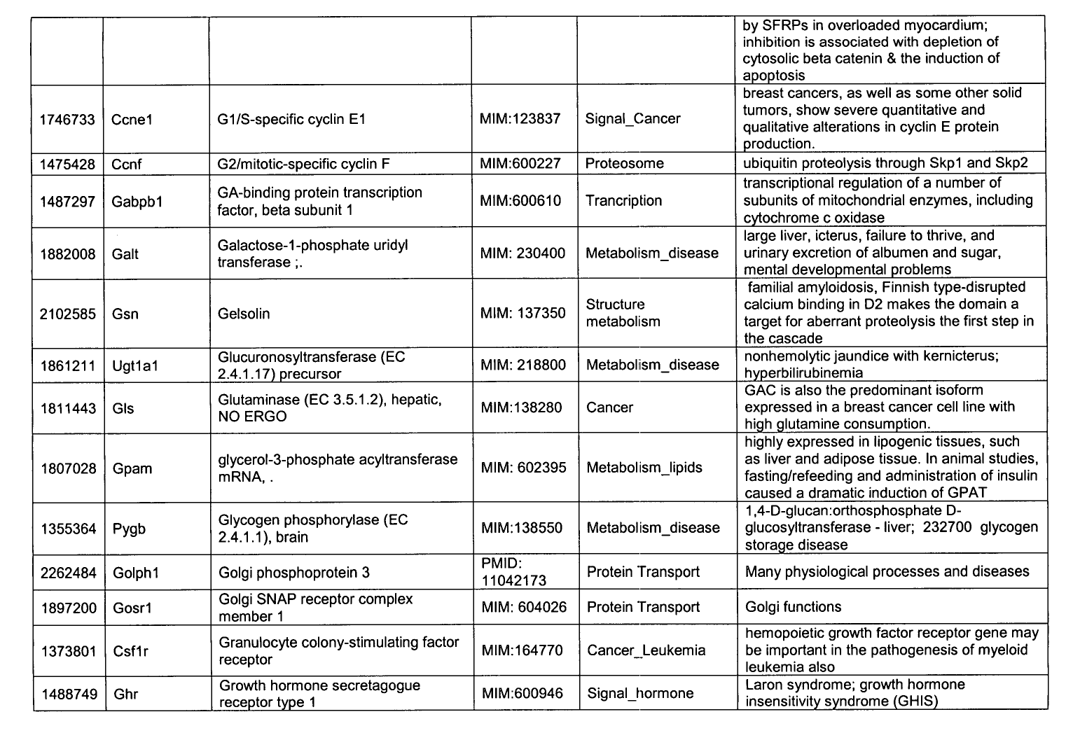

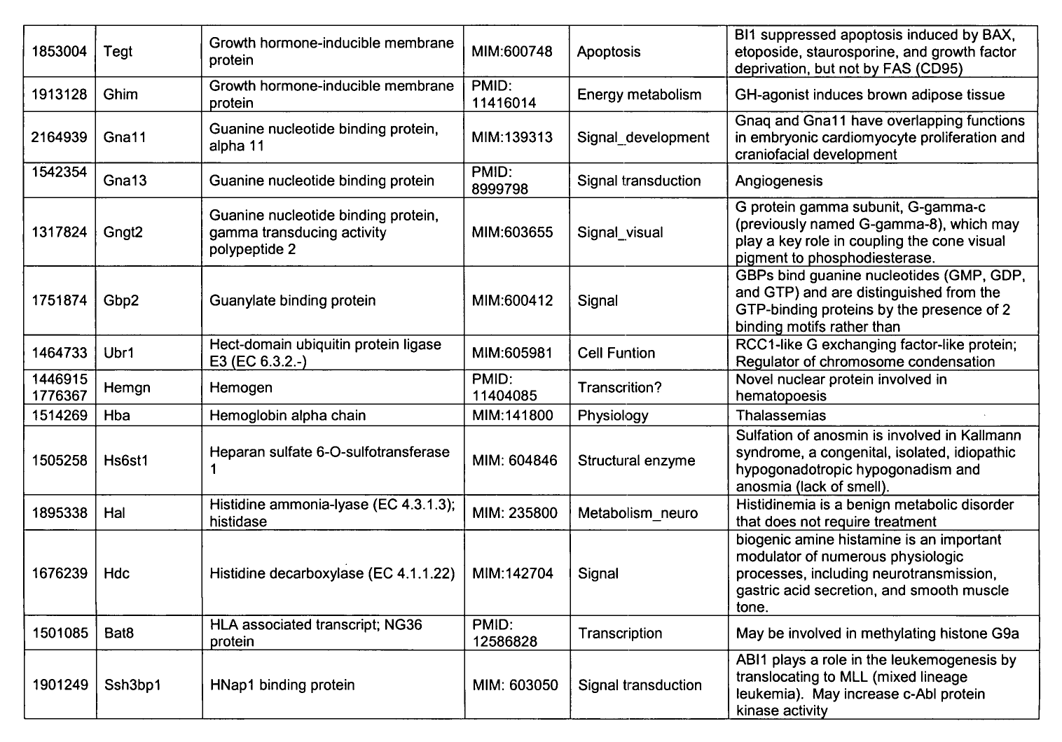

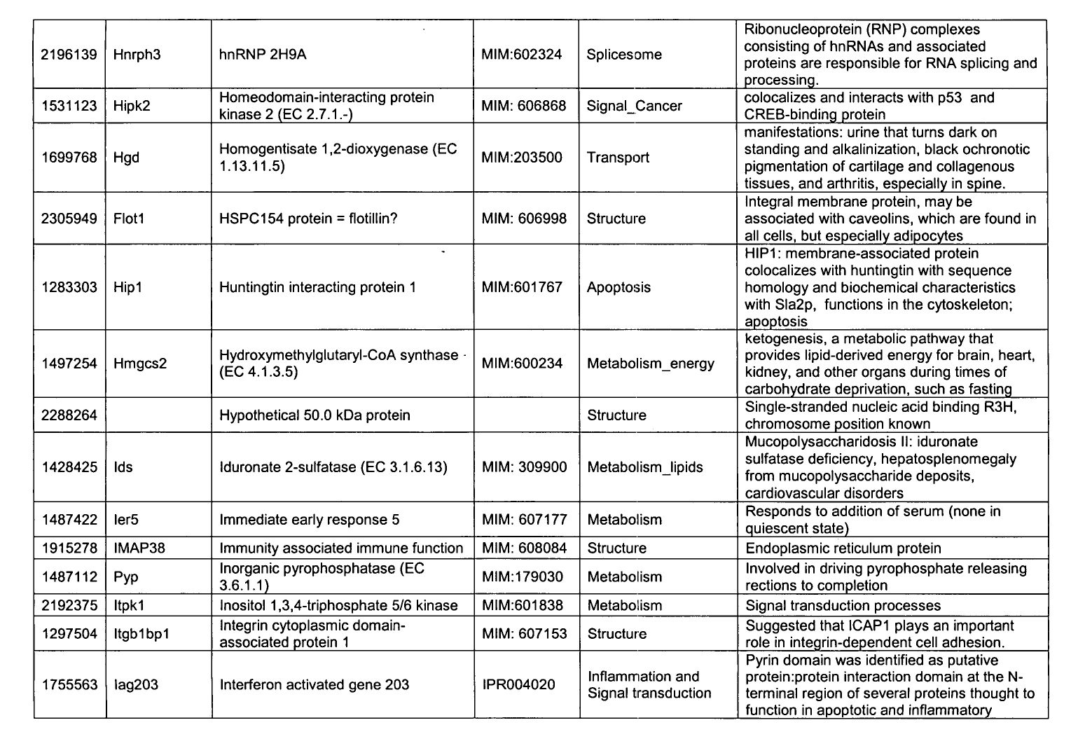

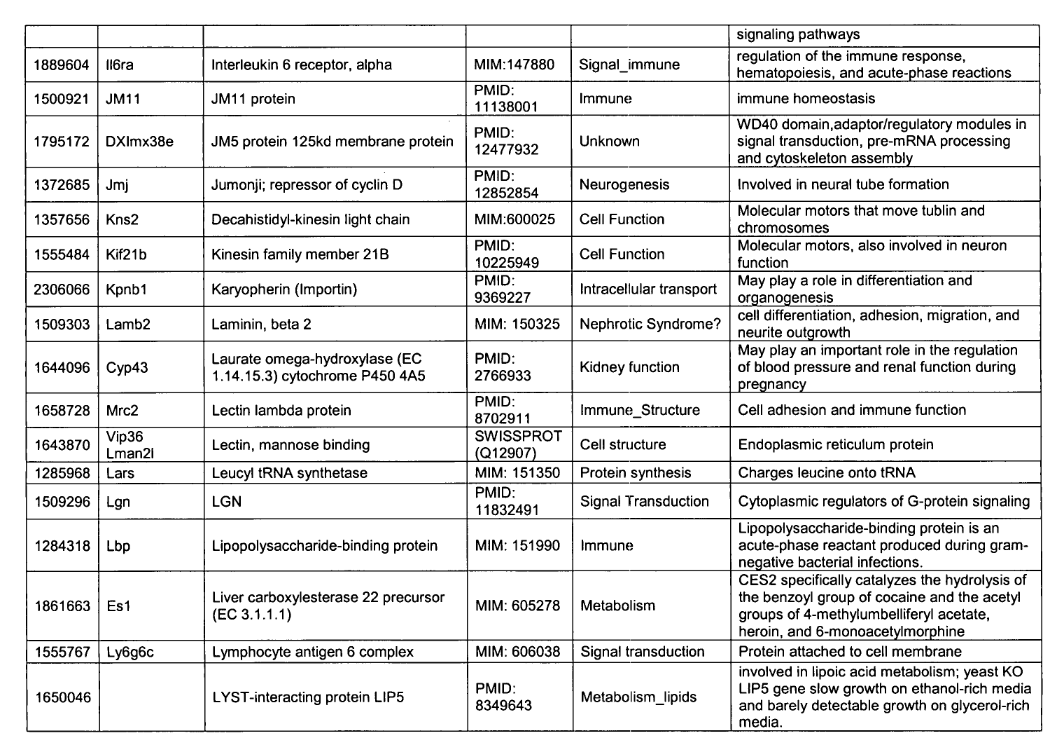

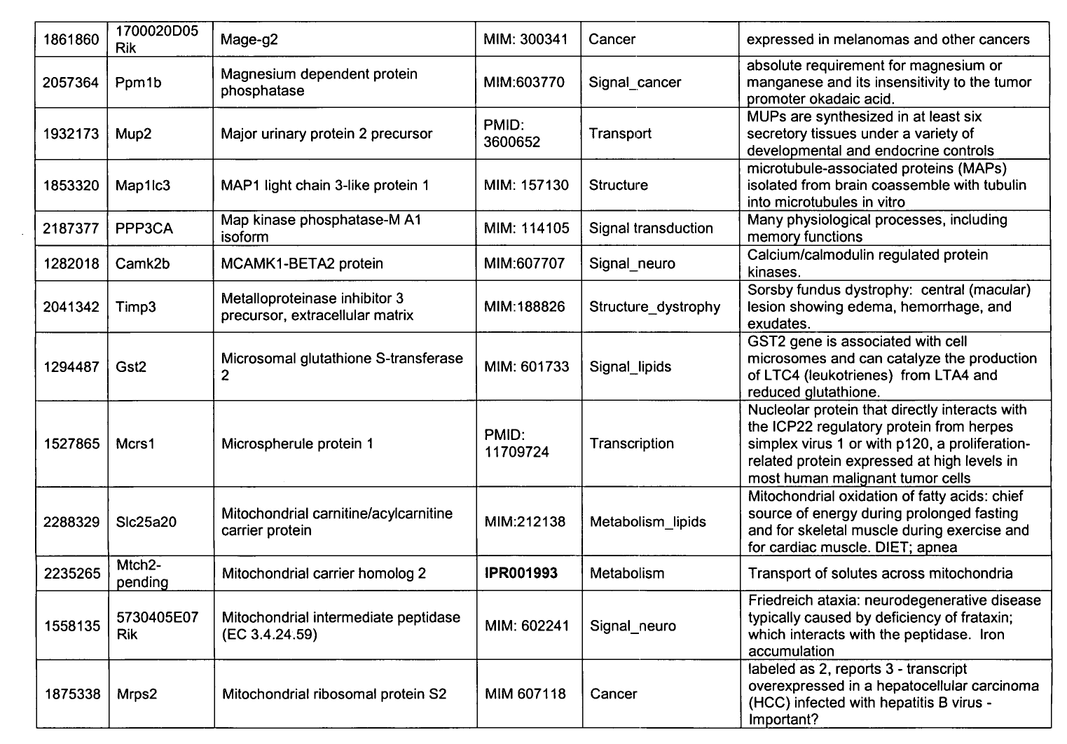

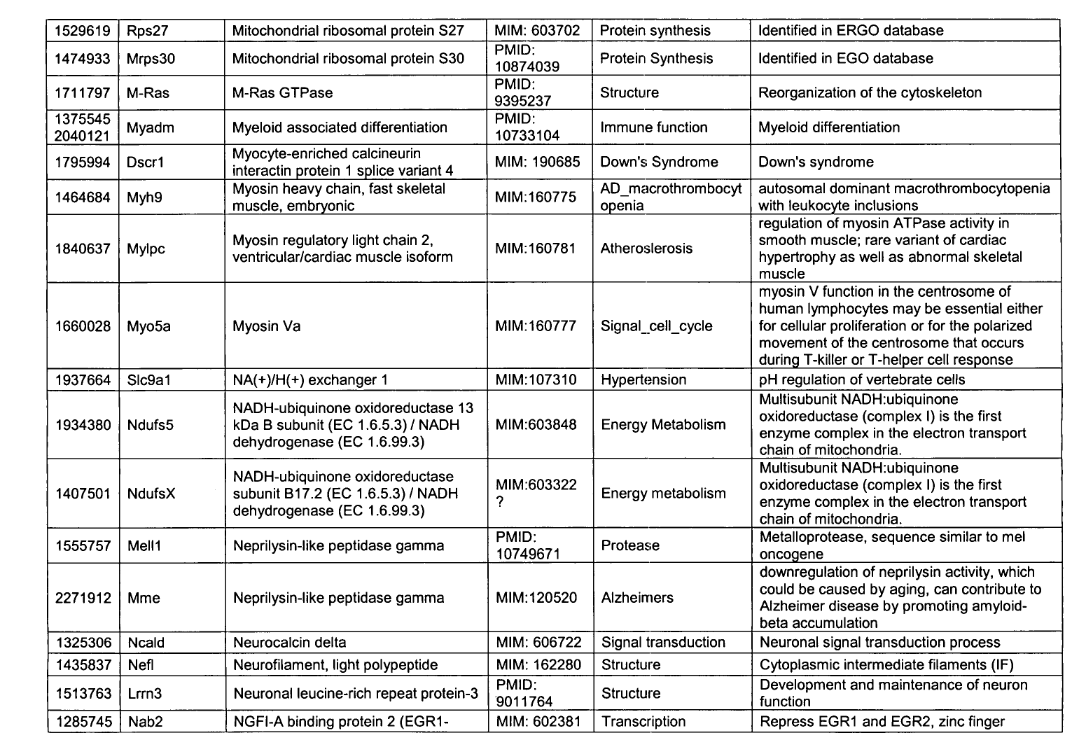

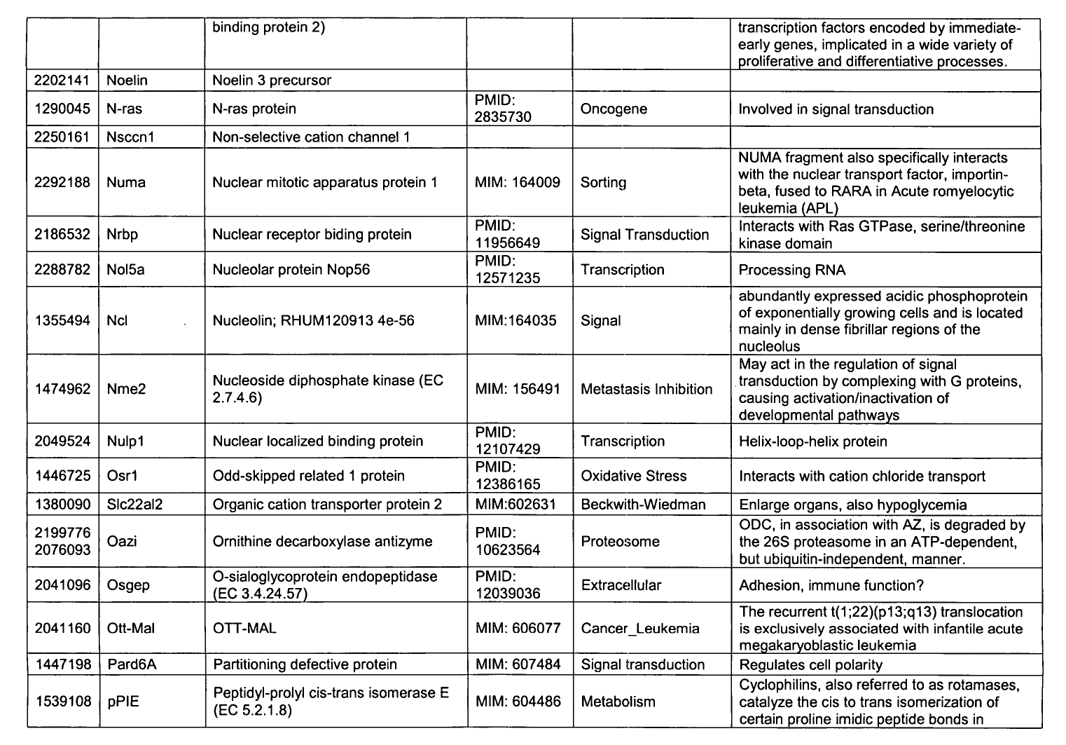

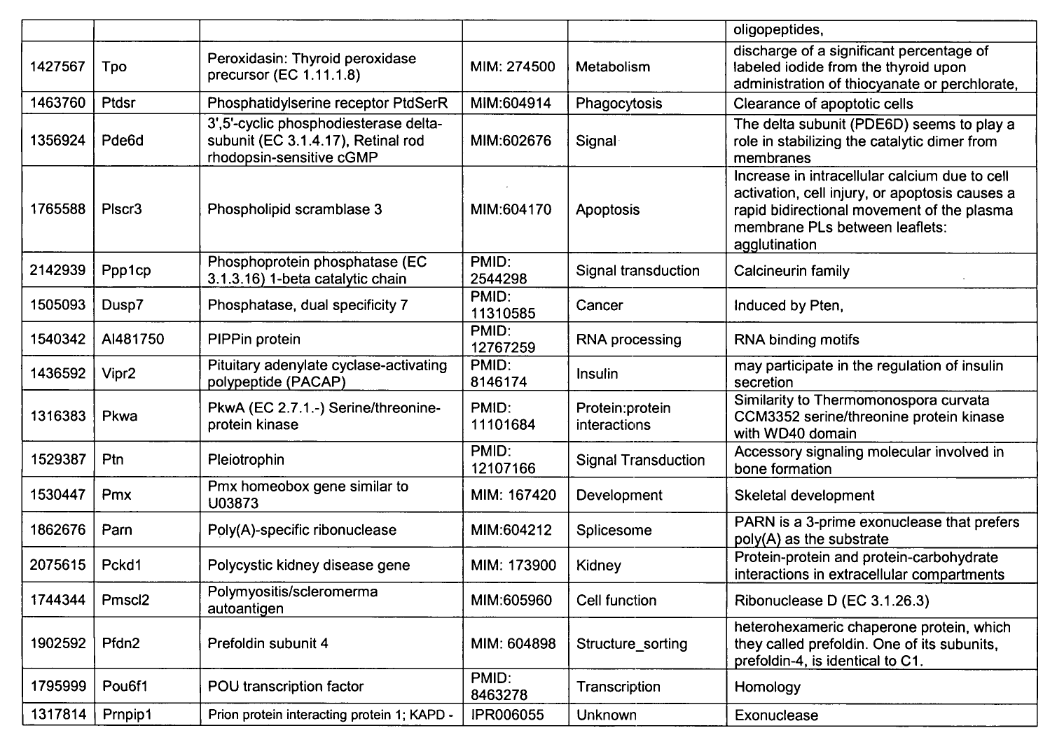

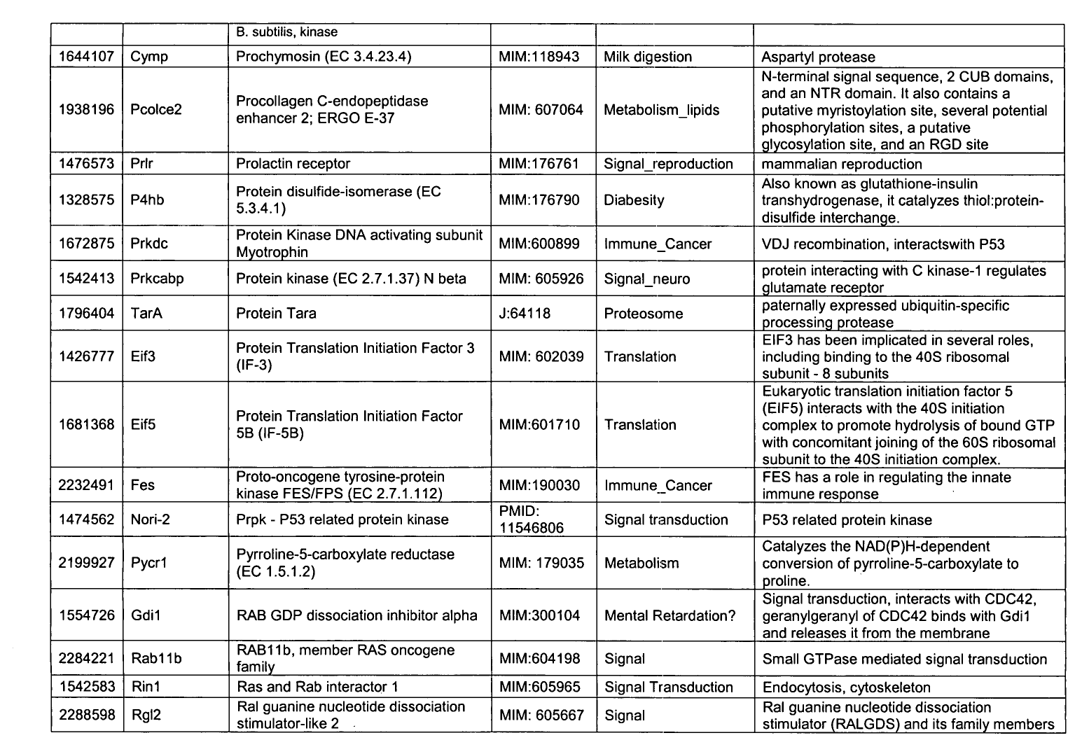

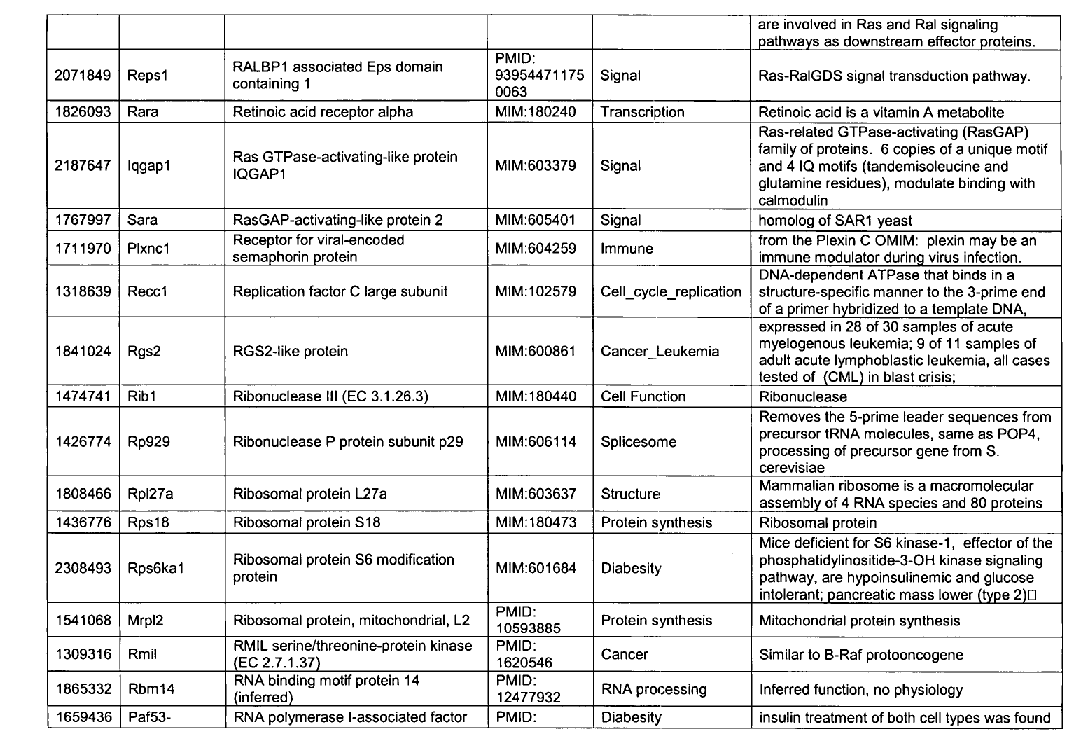

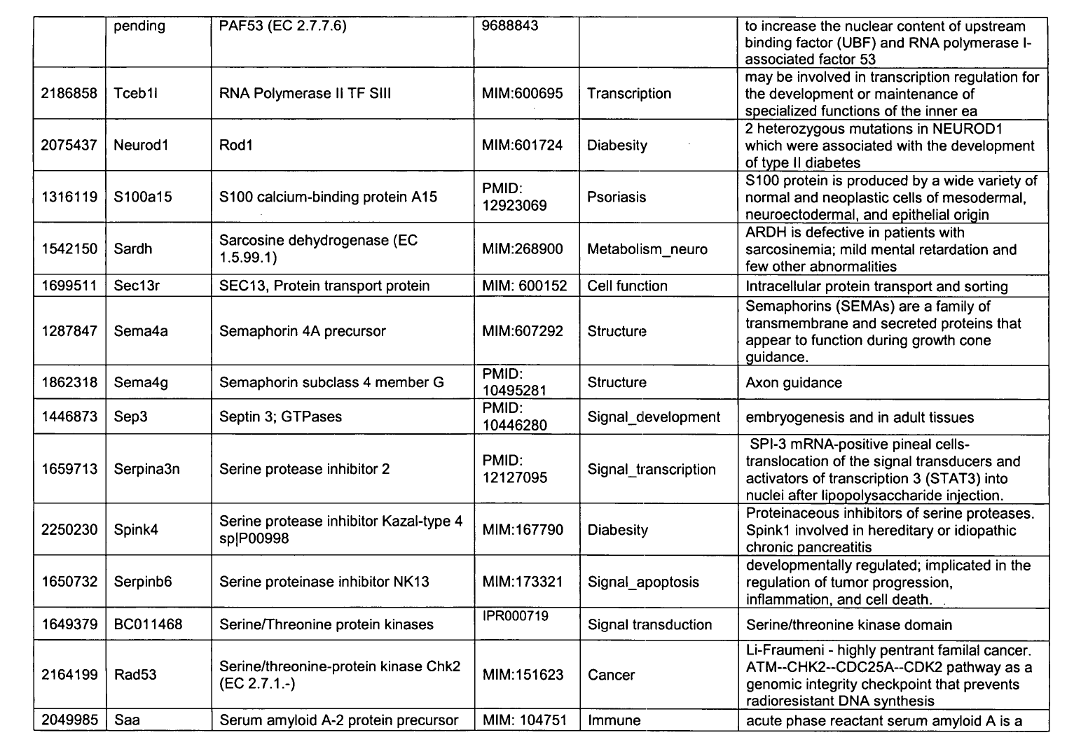

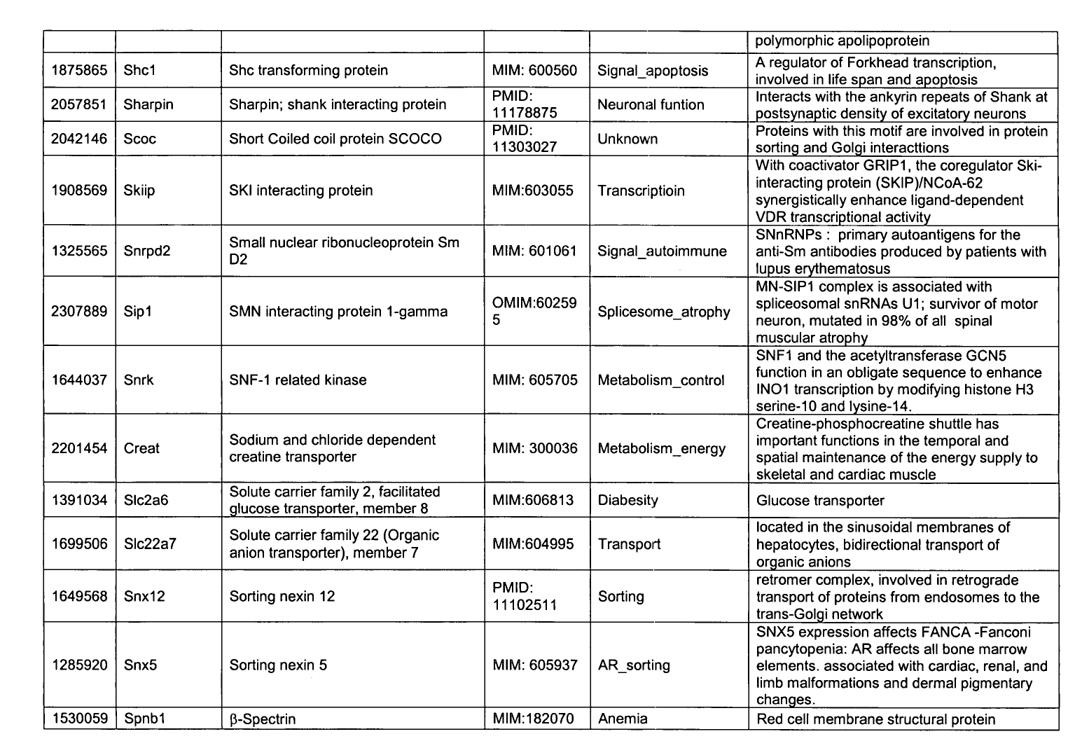

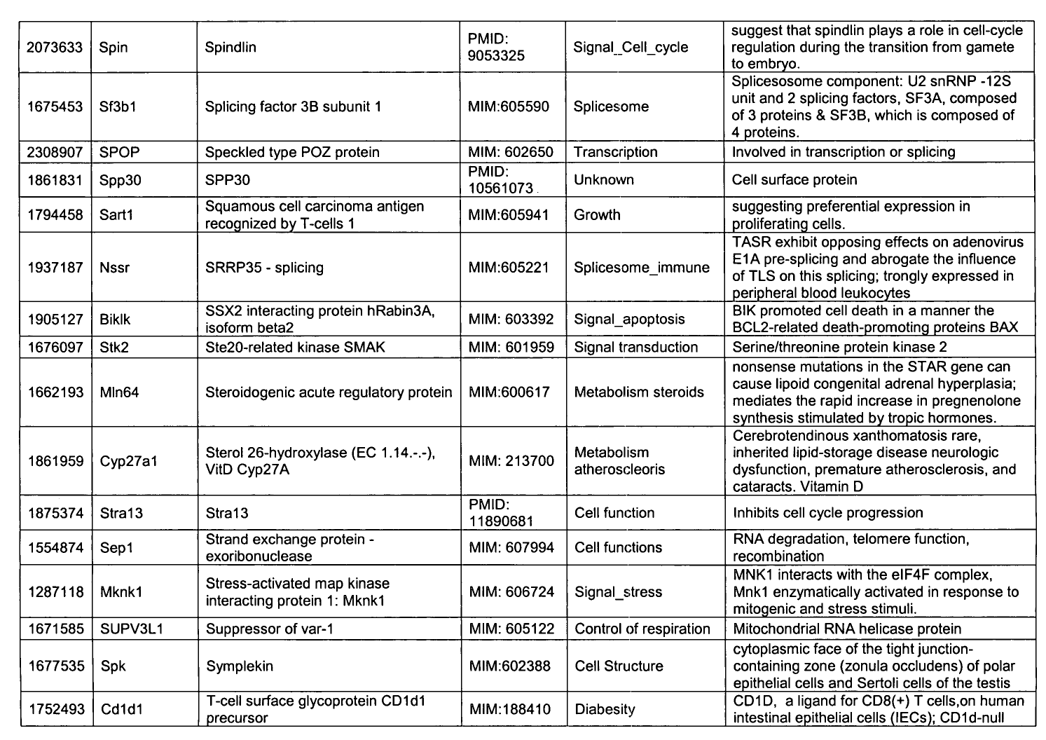

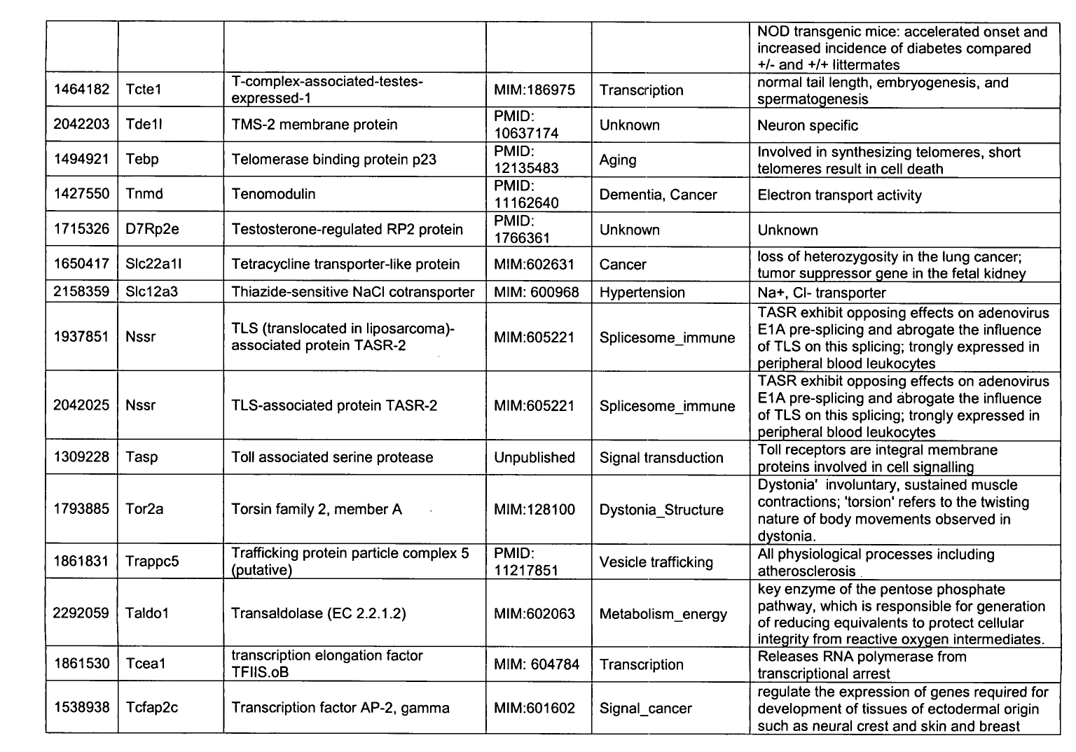

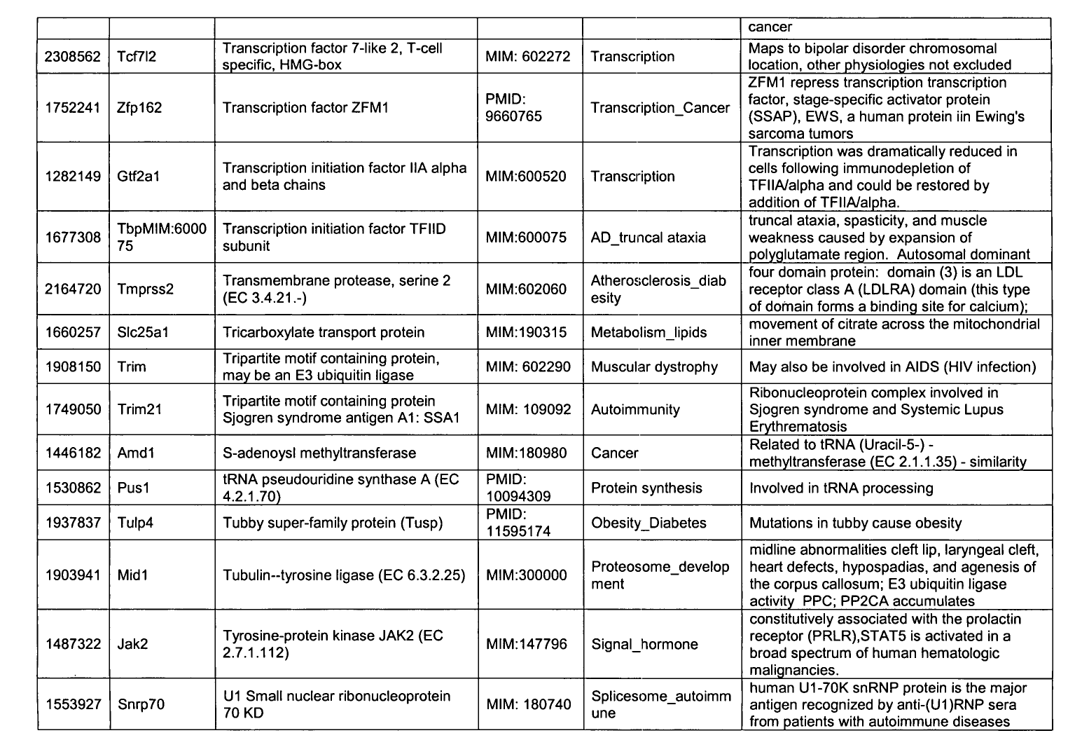

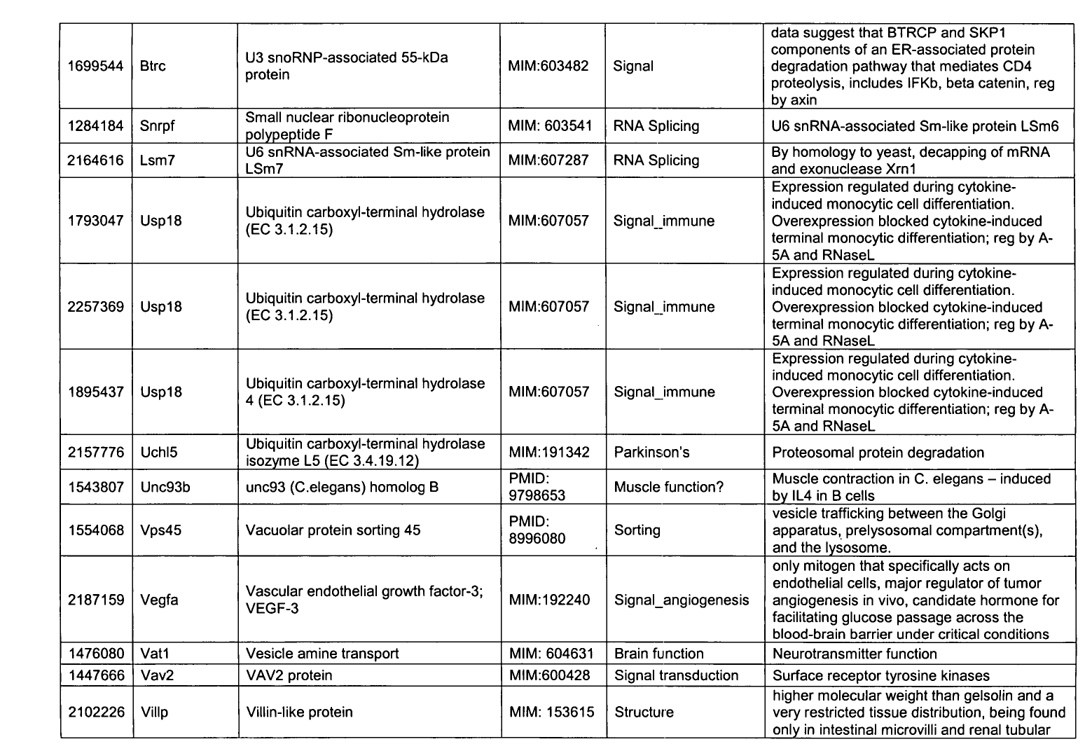

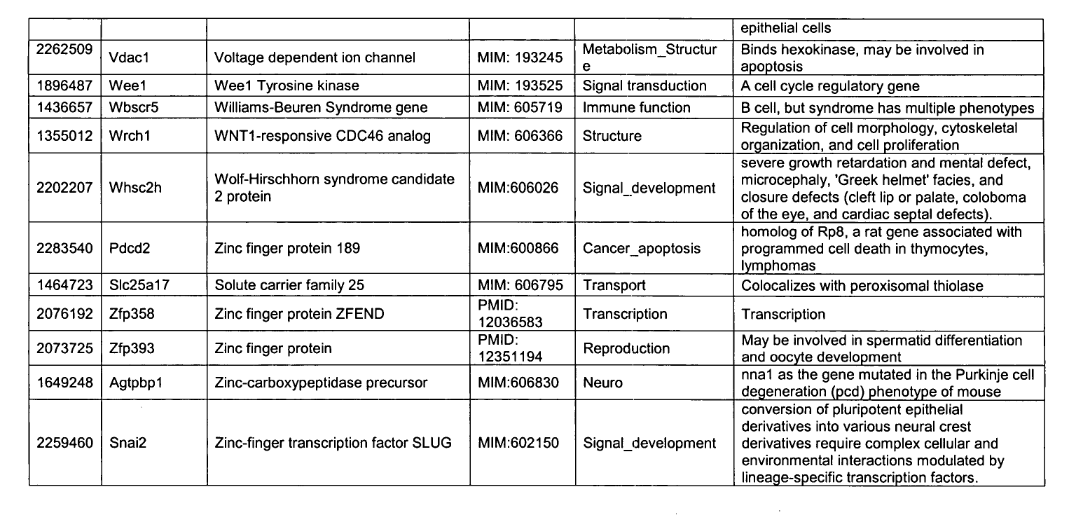

The map positions of the 376 genes of known function were determined and compared to quantitative trait loci (QTL) associated with various subphenotypes of diabetes and obesity (“diabesity”) (FIG. 3). Thirty-two (32) of the diet-, genotype-, and diet+genotype-regulated genes in liver mapped to diabesity QTL (FIG. 3). Genes mapping to weight gain or obesity QTLs were also determined (FIG. 3). An interval distance of ±10 cM from a given QTL was used, a distance consistent with the marker density employed in most QTL association studies.

Several of the gene products regulated by diet, genotype, or their interactions are associated with diabetes and/or are in pathways that alter phenotypes consistent with one of the conditions of diabetes (Table 3, Diabesity Association). Genes mapping to diabesity QTL and differently regulated are likely to cause differences in diabetes subphenotypes:

Camk2b (0.5 cM) maps near a non-insulin dependent diabetes (Nidd4n) QTL (2 cM) on chromosome 11. This gene product is a key regulator of insulin secretion by the pancreas but is also expressed in liver. The allele at Nidd4n plays a role in regulating insulin:glucose ratios.

Recc1, the large subunit of replication factor C is the ATP-dependent primer binding subunit of DNA polymerase, was found in the large complex with other DNA repair genes called BASC—the BRCA1 associated genome surveillance complex. Recc1 (39 cM) maps near Dbsty2, a QTL associated with plasma glucose levels and several obesity and body weight QTLs (Obq12, Bw8, Bwob) on chromosome 5 (29-44 cM).

Several other genes had more complex regulatory patterns but may play a role in causing differences in subphenotypes of diabesity:

Mapk1 (9.82 cM) maps near tally ho associated non-insulin dependent diabetes mellitus (Tanidd3, 5 cM) on chromosome 16. In adipocytes, Mapk1 regulates Foxc2, a transcription factor involved in regulating insulin responsiveness. An allele at Tanidd2 is linked to hyperglycemia.

Sf3b1, a gene encoding subunit 1 of the splicing factor 3B, maps on chromosome 1 (at 28.9 cM) near Dbsty1 (21 cM), a locus associated with body weight, plasma insulin and plasma glucose levels. Several genes involved in insulin metabolism, including the insulin receptor and attractin (the receptor of the agouti protein) are alternatively spliced producing variants with differential influences on insulin metabolism. Leiter's group has demonstrated that Dbsty1 interacts epistatically with Dbsty loci on chromosomes 5,12, and 15.

NeuroD is a transcription factor involved in insulin gene regulation in the pancreas. Mutations in this gene result in mature onset diabetes of the young (NeuroD=MODY6). NeuroD (46 cM) maps near a Type 2 diabetes mellitus (T2dm3) QTL (42.7) on chromosome 2 and 4 obesity or weight gain QTL (Mob7, Mob6, Obq3, Bw6). The allele at T2dm3 is linked with fasting plasma insulin levels.

Several other diet-regulated genes produce proteins that are considered markers of diabetes. For example, fibronectin and albumin are increased in serum of diabetics and their glycation state correlates with the severity of the disease. Enzymes such as glutaminase and histidine ammonia-lyase may play a role in serum pH balance through the production of ammonia. Glycerol 3-phosphate acyltransferase may alter triacylglycerol stores, a process which is nutritionally regulated and responsive to insulin.

Some individual genes have not been studied for a role in processes affected by diabetes, but members of their functional family have been linked to processes that are altered in that disease. Other genes and their products mapping to diabesity QTL have only a tentative association to conditions in diabetes—aquaporin, tricarboxylate transport protein, ankryin, metaloproteases, and debrins are examples although explanations can be developed for each of these potential candidates. Nevertheless, these genes are candidates for diabetes subphenotypes by virtue of their regulation by genotype or diet and their map position near QTL associated with diabetes symptoms.

The method and the newly identified diet-regulated disease-associated genes disclosed herein, may be used in a method to determine the susceptibility of an individual to a disease, wherein the disease involves one or more diet-regulated disease-associated polynucleotide identified by the method of the invention. The new method and the newly identified diet-regulated disease-associated genes may be used in a method for monitoring the progression of a disease by screening the individual for the presence and/or expression of a plurality of polynucleotides, and at a second date re-screening the individual for the expression of the same plurality of polynucleotides, wherein a change in polynucleotide expression corresponds to the desirable or undesirable progression of a disease. The invention includes a method for treating a subject so as to reduce the risk of the individual developing a diet-associated disease, by screening the individual and altering the expression of one or more diet-regulated disease-associated polynucleotides, or by altering diet, for example by feeding the individual particular amounts of combinations of nutrients to reduce the risk of the subject developing the disease.

Additional embodiments include arrays (Microarrays, cDNA arrays) for detecting gene expression in subjects, wherein the genes are genes identified by the methods disclosed herein. The invention encompasses arrays having at least one nucleotide (probe) that corresponds to and/or binds specifically to at least one polynucleotide that has been identified by the disclosed methods as a diet-regulated disease-associated gene. Such an array may contain a plurality of such probes or cDNAs or polynucleotides, for example it may contain at least 5, 10, 20, 40, 60 or at least 100 such probes. Arrays are well known and need not be discussed here, except to say that arrays made with combinations of probes identified by the methods of the invention may be used to screen individuals and populations for susceptibility to a disease and to monitor disease progression.

Additionally, antibody probes and arrays may equally be used to detect protein expression and to screen for the presence and amount of proteins expressed by diet-regulated disease-associated genes.

The methods disclosed herein, using arrays and other tools, may be used to screen populations to determine the presence and frequency of various diet-regulated disease-associated genes in a population or humans or animals. This information may be used to formulate foods (“medical foods” or “nutraceuticals”) that provide enhanced health benefits to various individuals and populations. For example, in-bred breeds of dog, say Labradors, may be analyzed using the methods of the invention, and susceptibility to various diseases, such as diabetes, may be determined. In this case, pet-foods may be formulated for that particular breed of dog to provide particular health benefits and to address this breed's particular needs. The same methods of food formulation may be done for any animals including farm animals, pet animals, and humans.

Genes Mapping to Obesity QTL

Genes mapping to obesity loci may contribute to subphenotypes of obesity. Other QTL for adiposity at various sites and total carcass lipid levels (for a comprehensive review see http://www.jax.org) were not included in our analyses since these parameters were not measured in this study. Many diabesity QTL overlap obesity QTL (FIG. 3) as would be expected for the diabesity phenotype. Associations with many specific molecular pathways influencing or involved in obesity and/or weight gain can be made for each of the other diet-, genotype-, and diet+genotype-regulated genes mapping to obesity and weight gain QTLs (FIG. 3).

Diabesity is a complex trait resulting from interactions between multiple genes and environmental factors. In humans, chronic exposure to excessive calories, deficiencies of micronutrients, and certain types of macronutrients induce obesity and diabetes in individuals, presumably without deleterious mutations in participating genes. These diseases therefore fit the common variant/common disease hypothesis proposed by Lander and Collins and colleagues. We have proposed that one or more of the gene products participating in development of chronic diseases will be regulated at least in part by diet since different macronutrients and excess calories are associated with almost all chronic diseases.

Although chronic diseases are multigenic in nature, much information regarding the pathways involved in disease development has been discovered by the study of rodent models with single gene defects or induced mutations (knockouts and transgenics) that mimic diabetes and/or obesity. Comprehensive reviews of the mouse models for insulin resistance and obesity have recently been published. The general conclusion from these reviews echoes the conclusion of Wolff that similar if not identical phenotypic expressions of a disease state can be reached by different metabolic routes. That is, alterations in many pathways can produce the same phenotype. Nevertheless, these genes and their variants may identify sets of pathways that collectively produce the specific diabetes subphenotypes and obesity pattern in one model of obesity and diabetes. That is, some of these pathways may also be involved in other models of diabesity if gene variants in the key regulatory or structural genes collectively produce expression changes similar to those observed in this specific model. Genes identified in the A^{vy}/A and A/a comparison may contribute to obesity or diabesity in humans if their regulation is altered in a similar manner. Although we and others identify candidate disease genes through gene expression analyses, changes in the activity of proteins encoded by gene specific mRNAs may also be associated with disease development. Polymorphisms within the coding sequences of candidate genes (cSNPs) that alter their function also contribute to disease development [54].

We found 601 hepatic genes or ESTs regulated in the same manner in replicate experiments with 376 genes having a known function. We used a cutoff ratio of 2.5 fold or greater or 1/2.5 fold or less to define regulation because of hybridization conditions and type of DNA array. Genes of all functional classes and types of regulation were differently expressed in this genotype comparison.

Quantitative trait loci are used to associate chromosomal regions with complex traits. There are now over 1700 QTL for disease, subphenotypes of disease, enzyme or protein levels, behavior, and other complex traits in mice (see http://www.jax.org). The limitations of using QTL data are that (i) they may be specific to the inbred strains analyzed, (ii) identify 20-30 cM regions of DNA, and (iii) often can not detect interactions with other loci. In addition, few mapping studies rigorously control or report diets; environment is known to have a large influence on the identification of QTL affecting complex traits, at least in plants. Our approach combines the strength of array technology with the power of genetics to identify potential causative genes. A key additional component of our approach is the rigorous control of diet composition and a timed feeding regimen that will allow for replication of the experiments.

Even with limitations of the current experiment (type of array, number of mice, single tissue source), the data presented herein identify potential novel candidate genes in many different functional pathways that may play a role in expression of subphenotypes of diabesity. Several of the genes that were found to be diet-regulated and mapped to diabetes QTL had previously been linked to specific pathways affected by or involved in diabetes. NeuroD, Camk2b, and Rgs2 are linked to Type 2 diabetes or are in pathways directly regulating insulin function. Fibronectin and albumin are used to monitor glycation levels in diabetics; however, their regulation and map position within diabesity loci suggest that they may play a more active role in evolution of the chronic disease state. Two genes which produce ammonia also mapped to QTL suggesting a potential mechanism for acid:base balance and alterations in nitrogen metabolism in diabetes. Other genes mapping to QTL from our screen can be associated with various alterations in metabolism found in diabesity. They become candidates for further testing.

Since obesity is a more “amorphous” phenotype, with adiposity, weight gain, and overall weight as the key phenotypic markers, it is more difficult to compare candidates identified in this screen with those found in other model organisms or humans. Genes analyzed in this screen that are regulated by diet, genotype, and genotype X diet that map to obesity QTL may be considered candidates for obesity development or severity.

The strategy described is a means to identify diet-, genotype-, and genotype X diet-regulated genes that cause or promote the development and severity of complex phenotypes. This approach can be applied to comparisons of each mutant model and its normal inbred parent or strain and to congenic siblings produced specifically for separating and combining QTL producing a complex phenotype. By comparing across the different genotypes fed the same diet, genotype-regulated genes can be identified. Similarly, by feeding two or more diets to mice with different genotypes, diet-regulated- and diet+genotype-regulated genes can be identified.

Understanding diabetes and obesity will require integration of knowledge from individual pathways that have been elucidated to date. However, inclusion of diet as a variable in a systems biology approach will also be necessary to fully explain complex phenotypes, almost all of which are influenced by environment, and specifically by dietary variables. This type of scientific study is called nutrigenomics or nutritional genomics. Knowledge of the interactions of diet and genotype will be needed when testing and treating these diseases in human populations.

Although various examples are given in this disclosure, they are for illustrative purpose only and in no way limit the scope of the invention.

### REFERENCES

Krum, H., New and emerging pharmacologic strategies in the management of chronic heart failure. Clin Cardiol, 2000. 23(10): p. 724-30.

Willett, W. C., Balancing life-style and genomics research for disease prevention. Science, 2002. 296(5568): p. 695-8.

Dauncey, M. J., et al., Nutrition-hormone receptor-gene interactions: implications for development and disease. Proc Nutr Soc, 2001. 60(1): p. 63-72.

Kaput, J., et al., Diet-disease interactions at the molecular level: an experimental paradigm. J Nutr, 1994. 124(8 Suppl): p. 1296S-1305S.

Park, E. I., et al., Lipid level and type alter stearoyl CoA desaturase mRNA abundance differently in mice with distinct susceptibilities to diet-influenced diseases. J Nutr, 1997. 127(4): p. 566-73.

Chanock, S., Candidate genes and single nucleotide polymorphisms (SNPs) in the study of human disease. Dis Markers, 2001. 17(2): p. 89-98.

Nowotny, P., J. M. Kwon, and A. M. Goate, SNP analysis to dissect human traits. Curr Opin Neurobiol, 2001. 11(5): p. 637-41.

Schork, N. J., D. Fallin, and J. S. Lanchbury, Single nucleotide polymorphisms and the future of genetic epidemiology. Clin Genet, 2000. 58(4): p. 250-64.

Enattah, N. S., et al., Identification of a variant associated with adult-type hypolactasia. Nat Genet, 2002. 30(2): p. 233-7.

Hollox, E. J., et al., Common polymorphism in a highly variable region upstream of the human lactase gene affects DNA-protein interactions. Eur J Hum Genet, 1999. 7(7): p. 791-800.

Harvey, C. B., et al., Lactase haplotype frequencies in Caucasians: association with the lactase persistence/non-persistence polymorphism. Ann Hum Genet, 1998. 62 (Pt 3): p. 215-23.

Williams, E. D. and J. D. Brooks, New molecular approaches for identifying novel targets, mechanisms, and biomarkers for prostate cancer chemopreventive agents. Urology, 2001. 57(4 Suppl 1): p. 100-2.

Group, L. S., Prevention of cardiovascular events and death with pravastatin in patients with coronary heart disease and a broad range of initial cholesterol levels. The Long-Term Intervention with Pravastatin in Ischaemic Disease (LIPID) Study Group. N Engl J Med, 1998. 339(19): p. 1349-57.

Risch, N., S. Ghosh, and J. A. Todd, Statistical evaluation of multiple-locus linkage data in experimental species and its relevance to human studies: application to nonobese diabetic (NOD) mouse and human insulin-dependent diabetes mellitus (IDDM). Am J Hum Genet, 1993. 53(3): p. 702-14.

Risch, N., Evolving methods in genetic epidemiology. II. Genetic linkage from an epidemiologic perspective. Epidemiol Rev, 1997. 19(1): p. 24-32.

Risch, N. and K. Merikangas, The future of genetic studies of complex human diseases [see comments]. Science, 1996. 273(5281): p. 1516-7.

Patterson, A. H., Damon, S. Hewitt, J. D., Zamir, D., Rabinowitch, H. D., Lincoln, S. E., Lander, E. S., and Tanksley, S. D., Mendelian Factors underlying quantitative traits in tomato: Comparison across species, generations, and environments. Genetics, 1991. 127: p. 181-197.

Lander, E. and L. Kruglyak, Genetic dissection of complex traits: guidelines for interpreting and reporting linkage results [see comments]. Nat Genet, 1995. 11(3): p. 241-7.

Frankel, W. N., Taking stock of complex trait genetics in mice. Trends Genet, 1995. 11(12): p. 471-7.

So, W. Y., et al., Genetics of type 2 diabetes mellitus. Hong Kong Med J, 2000. 6(1): p. 69-76.

Baier, L. J., et al., A calpain-10 gene polymorphism is associated with reduced muscle mRNA levels and insulin resistance. J Clin Invest, 2000. 106(7): p. R69-73.

Marx, J., Unraveling the causes of diabetes. Science, 2002. 296(5568): p. 686-9.

Elliott, T. S., et al., F1Fo-ATPase subunit e gene isolated in a screen for diet regulated genes. Biochem Biophys Res Commun, 1993. 190(1): p. 167-74.

Paisley, E. A., et al., Temporal-regulation of serum lipids and stearoyl CoA desaturase and lipoprotein lipase mRNA in BALB/cHnn mice. J Nutr, 1996. 126(11): p. 2730-7.

Swartz, D. A., et al., The e subunit gene of murine F1F0-ATP synthase. Genomic sequence, chromosomal mapping, and diet regulation. J Biol Chem, 1996. 271(34): p. 20942-8.

Wolff, G. L., et al., Caloric restriction abolishes enhanced metabolic efficiency induced by ectopic agouti protein in yellow mice. Proc Soc Exp Biol Med, 1999. 221(2): p. 99-104.

Reeves, P. G., F. H. Nielsen, and G. C. Fahey, Jr., AIN-93 purified diets for laboratory rodents: final report of the American Institute of Nutrition ad hoc writing committee on the reformulation of the AIN-76A rodent diet. J Nutr, 1993. 123(11): p. 1939-51.

1. A method for identifying diet-regulated disease-associated polynucleotides comprising the steps of:
(i) selecting at least two different inbred known genotypes (A and B), one of these genotypes (A) being more susceptible to a disease, and the other genotype (B) less susceptible to the same disease;
(ii) dividing each genotype into two groups (A1 and A2 and B1 and B2);
(iii) for each genotype, each group is fed a different diet (A1 is fed diet No.1 and A2 is fed diet No.2, and similarly for B1 and B2);
(iv) measuring gene expression and comparing expression across the strains that differ in either genotype or in diet, but not in both;
(v) analyzing the expression data so as to identify diet-regulated disease-associated genes.

2. The method of claim 1 further comprising comparing the diet-regulated disease-associated genes so identified with an independently-derived set of diet-regulated and/or disease associated QTLs.

3. The method of claim 1 wherein the disease is a diet-associated disease.

4. The method of claim 2 wherein gene expression is compared by comparing mRNA abundance.

5. A method for determining the susceptibility of an individual to a disease, wherein said disease involves a diet-regulated disease-associated polynucleotide, the method comprising: screening an individual for the presence and/or expression of a plurality of polynucleotides identified by the method of claim 1, wherein the pattern of expression of said plurality of polynucleotides corresponds with the susceptibility of an individual to a certain disease.

6. A method for monitoring the progression of a disease in a subject, the method comprising: at a first date, screening an individual for the presence and/or expression of a plurality of polynucleotides identified by the method of claim 1; at a second date re-screening the individual for the expression of the same plurality of polynucleotides, wherein a change in polynucleotide expression corresponds to the desirable or undesirable progression of a disease.

7. A method for treating a subject so as to reduce the risk of the individual developing a diet-associated disease, the method comprising: screening an individual for the presence and/or expression of a plurality of polynucleotides identified by the method of claim 1, wherein the pattern of expression of said plurality of polynucleotides corresponds with the susceptibility of an individual to a certain disease; and altering the expression of one or more diet-regulated disease-associated polynucleotides to reduce the risk of the subject developing the disease.

8. A method for treating a subject so as to reduce the risk of the individual developing a diet-associated disease, the method comprising: screening an individual for the presence and/or expression of a plurality of polynucleotides identified by the method of claim 1, wherein the pattern of expression of said plurality of polynucleotides corresponds with the susceptibility of an individual to a certain disease, and altering the diet of the individual so as to reduce the risk of the subject developing the disease.

9. A method for treating a subject so as to ameliorate a diet-associated disease, the method comprising: screening an individual for the presence and/or expression of a plurality of polynucleotides identified by the method of claim 1, wherein the pattern of expression of said plurality of polynucleotides corresponds with the susceptibility of an individual to a certain disease; and altering the expression of one or more diet-regulated disease-associated polynucleotides so as to affect an improvement in the progression of the disease.

10. A method for treating a subject so as to reduce the risk of the individual developing a diet-associated disease, the method comprising: screening an individual for the presence and/or expression of a plurality of polynucleotides identified by the method of claim 1, wherein the pattern of expression of said plurality of polynucleotides corresponds with the susceptibility of an individual to a certain disease, and altering the diet of the individual so as to affect an improvement in the progression of the disease.

11. A method for classifying a subject diagnosed with a disease so as to select appropriate drug(s) or dietary treatment(s) for treating the disease, the method comprising: screening an individual for the presence polynucleotides identified by the method of claim 1, wherein the presence of various nucleotides corresponds to a particular classification.

12. A method for formulating a food comprising: screening an in-bred population using the method of claim 1, determining the presence of one or more diet-regulated disease-associated genes in the population, determining which dietary elements are associated with altered activity of the genes, and formulating a food so as to appropriately alter the amount of the dietary elements in the food.

13. A food formulated using the method of claim 12.

14. An array comprising a plurality of nucleotide probes, wherein at least one nucleotide probe corresponds to a diet-regulated disease-associated gene identified by the method of claim 1.

15. The array of claim 13 comprising at least two nucleotide probes identified by the method of claim 1.

16. A method for identifying diet-regulated disease-associated polynucleotides, the method comprising the following steps:
a) comparing gene expression between two inbred strains in response to different diets, wherein one inbred strain is susceptible to a disease and the other inbred strain is not susceptible to the disease,
b) identifying those differentially expressed polynucleotides that overlap with independently-derived diet-regulated QTLs, and
c) analyzing the data to identify diet-regulated disease-associated polynucleotides.
